(19) United States
(12) Patent Application Publication
(10) Pub. No.: US 2024/0252593 A1
(43) Pub. Date: Aug. 1, 2024

(54) ASSESSING AND TREATING OBESITY

(71) Applicant: Mayo Foundation for Medical Education and Research, Rochester, MN (US)

(72) Inventors: Michael L. CAMILLERI, Rochester, MN (US); Andres J. ACOSTA, Rochester, MN (US); Paul A. DECKER, Rochester, MN (US); Jeanette E. ECKEL PASSOW, Pine Island, MN (US)

(21) Appl. No.: 18/562,949

(22) PCT Filed: May 20, 2022
(86) PCT No.: PCT/US22/30261

§ 371 Date: Nov. 21, 2023

Related U.S. Application Data
(60) Provisional application No. 63/191,588, filed on May 21, 2021.

(51) Int. Cl. A61K 38/26; A61K 31/137; A61K 31/36; A61K 31/485; A61P 3/04; C12Q 1/6851; C12Q 1/6883; G16B 20/00; G16H 10/40; G16H 50/20

(52) U.S. Cl. CPC A61K 38/26; A61K 31/137; A61K 31/36; A61K 31/485; A61P 3/04; C12Q 1/6851; C12Q 1/6883; G16B 20/00; G16H 10/40; G16H 50/20; C12Q 2600/106; C12Q 2600/156

(57) ABSTRACT

The present disclosure relates to methods and materials for assessing and/or treating obesity and/or obesity-related co-morbidities in mammals (e.g., humans). For example, methods and materials for using one or more interventions (e.g., one or more pharmacological interventions) to treat obesity and/or obesity-related co-morbidities in a mammal (e.g., a human) identified as being likely to respond to a particular intervention (e.g., a pharmacological intervention) are provided.

### CROSS-REFERENCE TO RELATED APPLICATIONS

This application claims the benefit of priority to U.S. Provisional Application No. 63/191,588 filed May 21, 2021, which is hereby incorporated by reference in its entirety for all purposes.

### STATEMENT REGARDING FEDERAL FUNDING

This invention was made with government support under DK067071 and DK114460 awarded by the National Institutes of Health. The government has certain rights in the invention.

### FIELD

The present disclosure is directed to methods and materials for assessing and/or treating obesity and obesity related co-morbidities (e.g., hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis, and atherosclerosis (coronary artery disease and/or cerebrovascular disease)) in mammals (e.g., humans). For example, this document provides methods and materials for determining an obesity analyte signature of a mammal. For example, this document provides methods and materials for determining an obesity phenotype of a mammal. For example, this document provides methods and materials for using one or more interventions (e.g., one or more pharmacological interventions) to treat obesity in a mammal (e.g., a human) identified as being likely to respond to a particular intervention (e.g., a pharmacological intervention such as, for example, a GLP-1 R analog or agonist).

### BACKGROUND

Obesity is a chronic, relapsing, multifactorial disease (Acosta et al., Clin. Gastroenterol. Hepatol., 15(5):631-49 e10 (2017); and Heymsfield et al., N. Engl. J. Med., 376(15):1492 (2017)), whose prevalence continues to increase worldwide (Ng et al., Lancet, 384(9945):P766-781 (2014); Collaborators G O, N. Engl. J. Med., 377:13-27 (2017); and Flegal et al., JAMA, 307(5):491-7 (2012)). In the United States alone, 69% of adults are overweight or obese (Flegal et al., 2012 JAMA 307:491-497). Estimated costs to the healthcare system are more than $550 billion annually. Increased severity of obesity correlates with a higher prevalence of the associated co-morbidities. Likewise, obesity increases the risk of premature mortality (Hensrud et al., 2006 Mayo Clinic Proceedings 81(10 Suppl):S5-10). Obesity affects almost every organ system in the body and increases the risk of numerous diseases including type 2 diabetes mellitus, hypertension, fatty liver disease, dyslipidemia, cardiovascular disease, and cancer. It is estimated that a man in his twenties with a BMI over 45 will have a 22% reduction (13 years) in life expectancy.

The complexities of obesity result in redundant and adaptive mechanisms to preserve energy; consequently, obesity is a remarkably heterogeneous disease, and sustained, successful outcomes with current treatment paradigms remain a challenge in clinical practice (Loos et al., Cell. Metab., 25(3):535-43 (2017); and MacLean et al., Obesity, 25 Suppl 1:S8-S16 (2017)). The heterogeneity among patients with obesity is particularly apparent in the treatment response to obesity interventions, such as diets, medications, devices, and surgery. Irrespective of the intervention, treatment response in highly variable; 30% of patients are poor responders (total body weight loss <5%), while 30% are regarded as positive responders, achieving clinically significant total body weight loss (>10%) (Heymsfield et al., N. Engl. J. Med., 376(15): 1492 (2017)). Despite considerable attempts to address predictors for weight loss, little is currently known about the predictors of response to obesity interventions (Loos et al., Cell. Metab., 25(3):535-43 (2017)).

Accordingly, there is an unmet need in the art to match subjects suffering from obesity to interventions most likely to produce an efficacious response (e.g., sustained weight loss) in a particular obese subject. Having the ability to identify which intervention(s) an obese patient is likely to respond to provides a unique and unrealized opportunity to provide an individualized approach in selecting obesity treatments. The materials and methods provided herein address this need.

### SUMMARY

In one aspect, provided herein is a method for treating obesity and/or one or more obesity-related co-morbidities in a mammal, the method comprising: (a) detecting the presence of a plurality of single nucleotide polymorphisms (SNPs) in a sample obtained from a mammal suffering from obesity, wherein the plurality of SNPs is selected from the group consisting of rs1664232, rs11118997, rs9342434, rs2335852, rs11020655, rs1885034, rs7277175, rs6923761, rs7903146, rs17782313, rs3813929, rs1047776 and any combination thereof; and (b) administering a GLP-1 agonist to the subject when the plurality of SNPs are detected in the sample, thereby treating the obesity and/or the one or more obesity-related co-morbidities. In some cases, the plurality of SNPs comprises rs1047776, rs17782313 and rs3813929. In some cases, the plurality of SNPs comprises rs11118997, rs1664232, rs6923761, rs9342434, rs2335852, rs11020655 and rs1885034. In some cases, the plurality of SNPs comprises rs11118997, rs1664232, rs6923761, rs9342434, rs2335852, rs1885034, rs11020655, rs1047776, rs17782313 and rs3813929. In some cases, the plurality of SNPs comprises rs1664232, rs11118997, rs9342434, rs2335852, rs11020655, rs1885034 and rs7277175. In some cases, the plurality of SNPs comprises rs1664232, rs11118997, rs9342434, rs2335852, rs11020655, rs1885034, rs7277175, rs7903146 and rs6923761. In some cases, the plurality of SNPs comprises rs1664232, rs11118997, rs9342434, rs2335852, rs11020655, rs1885034, rs7277175, rs7903146, rs6923761, rs1047776, rs17782313 and rs3813929. In some cases, the detecting is performed using an amplification, hybridization and/or sequencing assay. In some cases, the mammal suffering from obesity is a human. In some cases, the sample is selected from the group consisting of a blood sample, a saliva sample, a urine sample, a breath sample, and a stool sample. In some cases, the sample is a blood sample. In some cases, the GLP-1 agonist is selected from the group consisting of exenatide, liraglutide and semaglutide. In some cases, the GLP-1 agonist is liraglutide. In some cases, the method further comprises assessing gastric motor function of the mammal. In some cases, assessing the gastric motor function of the mammal comprises measuring the gastric emptying of the mammal. In some cases, a delay in gastric emptying for the mammal as compared to gastric emptying in a control selects the mammal for treatment with the GLP-1 agonist. In some cases, the one or more co-morbidities are selected from the group consisting of hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis and atherosclerosis (coronary artery disease and/or cerebrovascular disease).

In another aspect, provided herein is a method for assaying a sample obtained from a mammal suffering from obesity and/or one or more obesity-related co-morbidities, the method comprising detecting the presence of a plurality of single nucleotide polymorphisms (SNPs) in a sample obtained from the mammal, wherein the plurality of SNPs are selected from the group consisting of rs1664232, rs11118997, rs9342434, rs2335852, rs11020655, rs1885034, rs7277175, rs6923761, rs7903146, rs17782313, rs3813929, rs1047776 and any combination thereof. In some cases, the plurality of SNPs comprises rs1047776, rs17782313 and rs3813929. In some cases, the plurality of SNPs comprises rs11118997, rs1664232, rs6923761, rs9342434, rs2335852, rs11020655 and rs1885034. In some cases, the plurality of SNPs comprises rs11118997, rs1664232, rs6923761, rs9342434, rs2335852, rs1885034, rs11020655, rs1047776, rs17782313 and rs3813929. In some cases, the plurality of SNPs comprises rs1664232, rs11118997, rs9342434, rs2335852, rs11020655, rs1885034 and rs7277175. In some cases, the plurality of SNPs comprises rs1664232, rs11118997, rs9342434, rs2335852, rs11020655, rs1885034, rs7277175, rs7903146 and rs6923761. In some cases, the plurality of SNPs comprises rs1664232, rs11118997, rs9342434, rs2335852, rs11020655, rs1885034, rs7277175, rs7903146, rs6923761, rs1047776, rs17782313 and rs3813929. In some cases, the detecting is performed using an amplification, hybridization and/or sequencing assay. In some cases, the mammal suffering from obesity is a human. In some cases, the sample is selected from the group consisting of a blood sample, a saliva sample, a urine sample, a breath sample, and a stool sample. In some cases, the sample is a blood sample.

In yet another aspect, provided herein is a system for determining an obesity phenotype of a mammal suffering from obesity, the system comprising: (a) one or more processors; (b) one or more memories operatively coupled to at least one of the one or more processors and having instructions stored thereon that, when executed by at least one of the one or more processors, cause the system to: (i) identify the presence, absence or level of a plurality of gastrointestinal (GI) peptides, a plurality of metabolites, and/or a plurality of genetic variants in a sample obtained from a mammal suffering from obesity, thereby generating an analyte signature for the sample; (ii) populate a predictive machine learning model with the analyte signature of step (i); and (iii) utilize the predictive machine learning model to predict an obesity phenotype of the mammal suffering from obesity based on the analyte signature of the sample; and (c) one or more instruments in communication with at least one of the one or more processors, wherein the instruments, upon receipt of instructions sent by the at least one of the one or more processors, perform steps (i)-(iii). In some cases, the predictive machine learning model is selected from the group consisting of least absolute shrinkage and selection operator (LASSO) regression, a classification and regression tree (CART) model, and a gradient boosting machine (GBM) model. In some cases, the obesity phenotype is selected from the group consisting of abnormal satiation (hungry brain), abnormal satiety (hungry gut); hedonic eating (emotional hunger) and slow metabolism (slow burn). In some cases, utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some cases, utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some cases, the mammal suffering from obesity is a human. In some cases, the sample is selected from the group consisting of a blood sample, a saliva sample, a urine sample, a breath sample, and a stool sample. In some cases, the sample is a blood sample. In some cases, the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide. In some cases, the plurality of metabolites is selected from the group consisting of a bile acid, a neurotransmitter, an amino compound and a fatty acid. In some cases, the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine. In some cases, the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLP1R, PLXNA1, EYS, PTPRN2, PANX1, FRMD6, PCNT and BBS1. In some cases, the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577. In some cases, the one or more memories operatively coupled to the at least one of the one or more processors and having instructions stored thereon that, when executed by at least one of the one or more processors, further cause the system to populate the predictive learning model with data concerning the gastric motor function, resting energy expenditure (REE), one or more measures of appetite, results on behavioral questionnaires or any combination thereof of the subject suffering from obesity. In some cases, the gastric motor function is determined by measuring gastric emptying of the mammal. In some cases, the gastric emptying is measured using scintigraphy. In some cases, the REE of the mammal is measured by indirect calorimetry. In some cases, the behavioral questionnaire is a Hospital Anxiety and Depression Scale (HADS) questionnaire. In some cases, the one or more measures of appetite are selected from the group consisting of calories to fullness (CTF), maximum tolerated calories (MTC) and intake calories at an ad libitum buffet meal.

In still another aspect, provided herein is a method for treating obesity in a mammal, the method comprising: identifying the presence, absence or level of a plurality of GI peptides, a plurality of metabolites, and/or a plurality of genetic variants in a sample obtained from a mammal suffering from obesity, thereby generating an analyte signature for the sample; populating a predictive machine learning model with the analyte signature of step (a); utilizing the predictive machine learning model to predict an obesity phenotype of the mammal based on the analyte signature of the sample obtained from the mammal, wherein the obesity phenotype is selected from the group consisting of abnormal satiation (hungry brain), abnormal satiety (hungry gut); hedonic eating (emotional hunger) and slow metabolism (slow burn); and administering an intervention based on the obesity phenotype predicted in step (c). In some cases, the predictive machine learning model is selected from the group consisting of least absolute shrinkage and selection operator (LASSO) regression, a classification and regression tree (CART) model, and a gradient boosting machine (GBM) model. In some cases, utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some cases, utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. In some cases, the mammal suffering from obesity is a human. In some cases, the sample is selected from the group consisting of a blood sample, a saliva sample, a urine sample, a breath sample, and a stool sample. In some cases, the sample is a blood sample. In some cases, the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide. In some cases, the plurality of metabolites is selected from the group consisting of a bile acid, a neurotransmitter, an amino compound and a fatty acid. In some cases, the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine. In some cases, the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLP1R, PLXNA1, EYS, PTPRN2, PANX1, FRMD6, PCNT and BBS1. In some cases, the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577. In some cases, the method further comprises populating the predictive learning model with data concerning the gastric motor function, resting energy expenditure (REE), one or more measures of appetite, results on behavioral questionnaires or any combination thereof of the subject suffering from obesity. In some cases, the gastric motor function is determined by measuring gastric emptying of the mammal. In some cases, the gastric emptying is measured using scintigraphy. In some cases, the REE of the mammal is measured by indirect calorimetry. In some cases, the behavioral questionnaire is a Hospital Anxiety and Depression Scale (HADS) questionnaire. In some cases, the one or more measures of appetite are selected from the group consisting of calories to fullness (CTF), maximum tolerated calories (MTC) and intake calories at an ad libitum buffet meal. In some cases, the intervention is selected from the group consisting of a pharmacological intervention, a surgical intervention, a weight loss device, a diet intervention, a behavior intervention and a microbiome intervention. In some cases, the obesity phenotype is abnormal satiation (hungry brain) and the intervention is a pharmacological intervention, wherein the pharmacological intervention is phentermine-topiramate pharmacotherapy. In some cases, the obesity phenotype is abnormal satiety (hungry gut) and the intervention is a pharmacological intervention, wherein the pharmacological intervention is a GLP-1 agonist. In some cases, the GLP-1 agonist is selected from the group consisting of exenatide, liraglutide and semaglutide. In some cases, the obesity phenotype is hedonic eating (emotional hunger), and the intervention is a pharmacological intervention, wherein the pharmacological intervention is naltrexone-bupropion pharmacotherapy. In some cases, the obesity phenotype is slow metabolism (slow burn), and the intervention is a pharmacological intervention, wherein the pharmacological intervention is phentermine pharmacotherapy.

### BRIEF DESCRIPTION OF THE FIGURES

FIG. 1 illustrates obesity pathophysiology based on energy balance and key components that contribute to human obesity.

FIG. 2 illustrates how obesity phenotypes were identified by an unsupervised principal component analysis. A principal component analysis was performed in a new cohort of 120 participants with obesity that completed all the food intake and energy expenditures tests, described in the methods section. The PCA confirmed the key four latent dimension of obesity: hungry brain—abnormal satiation; hungry gut—abnormal satiety/gastric emptying; emotional hunger—abnormal hedonic eating/anxiety; and slow burn—abnormal predicted resting energy expenditure.

FIG. 3 illustrates the distribution of participants based on pathophysiological phenotypes in 120 patients with obesity (BMI>30 kg/m2). hungry brain—abnormal satiation, hungry gut—abnormal satiety, emotional hunger, slow burn—abnormal metabolism, mixed (25.8%) and other, that is 10.8% in whom none of the previously identified phenotypes was observed.

FIGS. 4A-4B illustrates a case-control prospective observation of obesity management with anti-obesity pharmacotherapy in a multidisciplinary weight management program comparing phenotype-guided pharmacotherapy to non-phenotype guided pharmacotherapy. FIG. 4A shows the total body weight loss (TBWL), while FIG. 4B shows the percentage of treatment responders.

FIG. 5 illustrates a decision tree, performance plot and table with performance summary for Prediction of Hungry Brain Phenotype (i.e., abnormal satiation) and/or calories intake using machine learning algorithms (CART or GBM).

FIG. 6 illustrates a decision tree, performance plot and table with performance summary for Prediction of Hungry Gut Phenotype (i.e., abnormal satiety) and/or calories intake using machine learning algorithms (CART or GBM).

FIG. 7 illustrates a decision tree, performance plot and table with performance summary for Prediction of Emotional Hunger Phenotype (i.e., abnormal hedonic eating) and/or calories intake using machine learning algorithms (CART or GBM).

FIG. 8 illustrates a decision tree, performance plot and table with performance summary for Prediction of Slow Burn Phenotype (i.e., abnormal metabolism) and/or calories intake using machine learning algorithms (CART or GBM).

FIG. 9 illustrates Manhattan plot from a genome-wide association study (GWAS) for gastric emptying of solids in obesity. The horizontal line shows the threshold for statistically significant association (p<1×10-5). Significant SNPs are labeled based on the nearest gene.

FIG. 10 illustrates the study design of the randomized, placebo-controlled trial of liraglutide with 82 participants with obesity (BMI≥30 kg/m²) as described in Example 5.

FIGS. 11A-11C illustrates the relationship of change in GE T1/2 and weight loss over 16 weeks of treatment for all the patients (FIG. 11A), liraglutide-treated patients (FIG. 11B) and placebo-treated patients (FIG. 11C).

FIG. 12 illustrates the weight loss at 16 weeks after 16 weeks of liraglutide based on baseline GE T1/2.

FIGS. 13A-13B illustrate weight loss after 16 weeks of liraglutide based on baseline GE T1/2 (FIG. 13A) as well as the fastest quartile GE T1/2 (FIG. 13B).

FIGS. 14A-14B illustrate that the alleles of rs6923761 (GLP-1 receptor) and change in weight (FIG. 14A) or change in GE T1/2 (FIG. 14B).

FIGS. 15A-15B illustrate that the alleles of rs7903146 (TCF7L2) and change in weight (FIG. 15A) or the effect of liraglutide on end of study weight of the CC genotype of rs7903146 (TCF7L2) by least square means based on rank scale, adjusted for baseline weight and sex (FIG. 15B).

FIGS. 16A-16B illustrate the effect of liraglutide on GE T1/2 (FIG. 16A) or max tolerated kCal (FIG. 16B) by least square means based on rank scale, adjusted for baseline weight and sex for the alleles of rs7903146 (TCF7L2).

FIG. 17 illustrates the study protocol utilized in the experiments described in Example 6.

FIG. 18 shows the flow chart for the study conducted in Example 6 with 182 adults assessed for eligibility, 136 randomized, and 124 completing the 16-week treatment trials (65 placebo and 59 liraglutide).

FIGS. 19A and 19B illustrate the effect of liraglutide or placebo treatment on gastric emptying T1/4 and T1/2 at 5 and 16 weeks (FIG. 19A) or the relationship of change in gastric emptying T1/2 to change in weight at 5 and 16 weeks in the liraglutide or placebo groups and the fastest quartile gastric emptying at baseline in the liraglutide (FIG. 19B).

FIGS. 20A and 20B illustrate weight loss for the liraglutide group compared to the placebo group at 5 weeks and at 16 weeks (FIG. 20A) or the volume to comfortable fullness, calories consumed during an ad libitum meal and maximum tolerated volume (p<0.001) at 16 weeks in the liraglutide group compared to the placebo group as documented by the changes from baseline (FIG. 20B).

FIG. 21 illustrates Spearman correlations showing the associations of gastric emptying T_{1/2} at 5 and 16 weeks and weight loss with treatment in the liraglutide or placebo groups.

FIG. 22 illustrates the correlation of GES T1/2 at 16 weeks and weight loss over the 16-week study period, but no significant correlation at 5 weeks in the liraglutide treatment group.

FIGS. 23A-23B illustrate the pharmacogenomic effects of SNP variants in GLP1R (FIG. 23A) and TCF7L2 (FIG. 23B) on responses to liraglutide of phenotypes related to obesity.

FIG. 24 illustrates total body weight loss percentage (TBWL %) between rapid gastric emptying (rapid GE) and patients with normal/slow GE for subjects treated with semaglutide.

FIG. 25 shows ROC curve evaluating variables included in the parsimonious model associated with weight loss >4 kilograms at 16 weeks in all patients.

FIG. 26 illustrates an ROC curve evaluating weight loss of >4 kg at 16 weeks in the liraglutide group only using baseline GES T1/2, week 5 GES T1/2, and meal total kcal at 16 weeks. Area under the curve=0.814. GES T1/2: gastric emptying of solids time to half emptying.

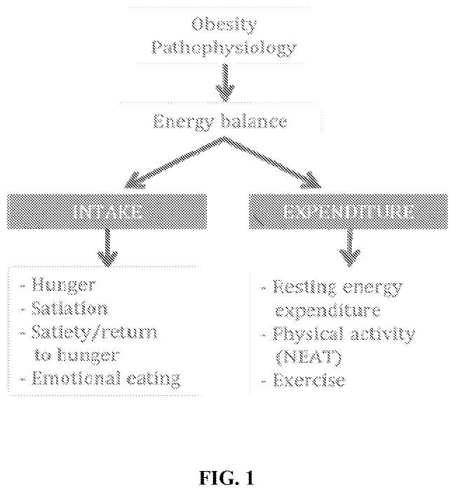

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e., can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

As used herein, the term “Calorie” or “kcal” can be used interchangeably and can generally refer to 1 Calorie (with a capital “C”) equaling 1 kcal, or 1000 calories (lower case “c”).

The term “weight loss” as used herein can refer to a reduction of the total body mass, due to a mean loss of fluid, body fat or adipose tissue and/or lean mass, namely bone mineral deposits, muscle, tendon, and other connective tissue.

The terms “ad libitum diet” as used herein refer to a diet where the amount of daily calories intake of a subject is not restricted to a particular value. A subject following an ad libitum diet is free to eat till satiation (or fullness).

The term “energy density” as used herein can refer to the amount of energy, as represented by the number of calories, in a specific weight of food.

The term “nutrient density” as used herein can refer to the balance of beneficial nutrients in a food (like vitamins, minerals, lean protein, healthy fats and fiber) compared with nutrients to limit (like saturated fat, sodium, added sugars and refined carbohydrates). Nutrient density can also refer to the amount of beneficial nutrients in a food product in proportion to e.g., energy content, weight or amount of detrimental nutrients. The terms such as nutrient rich and micronutrient dense can also refer to similar properties.

The terms “Glucagon-like peptide-1 receptor agonist” or “GLP-1 receptor agonist” as used herein can be used interchangeably with the terms “GLP-1 agonist” or “GLP-1 analog”. Said terms can also be referred to as incretin mimetics. All of the aforementioned terms can refer to agents that act as agonists of the GLP-1 receptor and can work by activating the GLP-1 receptor.

The term “postprandial satiety” as used herein can be interchangeably with “hungry gut” or “satiety” and refers to the sensation of fullness after a meal termination that perdures through time until hunger returns. Postprandial satiety may overlap with hunger or desire to eat.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control. In addition, the materials, methods, and examples are illustrative only and not intended to be limiting.

### Overview

### GLP-1 Receptor Analog Response Predictor Assays

Provided herein are methods and systems for predicting the response of an obese mammal to a GLP-1 agonist or analog, selecting an obese mammal for treatment with a GLP-1 agonist or analog and/or treating said obese mammal with a GLP-1 agonist or analog. In some cases, obesity and/or one or more obesity related co-morbidities are treated using the GLP-1 agonist or analog. Examples of weight-related or obesity-related co-morbidities include, without limitation, any obesity-related co-morbidity known in the art, such as, for example, hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis, and atherosclerosis (coronary artery disease and/or cerebrovascular disease). In one embodiment, provided herein is a method for assaying a sample obtained from a mammal suffering from obesity and/or an obesity-related co-morbidity, the method comprising detecting the presence of a plurality of single nucleotide polymorphisms (SNPs) in a sample obtained from the mammal suffering from obesity. In some cases, the assay is used to determine the obesity phenotype of the mammal suffering from obesity and/or an obesity-related co-morbidity. In one embodiment, the assay is used to determine if the mammal suffering from obesity possesses a hungry gut (e.g., abnormal postprandial satiety) obesity phenotype. In some cases, if a plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) are detected in a sample obtained from the obese mammal, said obese mammal is diagnosed with a hungry gut obesity phenotype. In some cases, the assay is used to predict the responsiveness of the mammal to a specific pharmacological intervention. In some cases, if a plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) are detected in a sample obtained from the obese mammal, said obese mammal is predicted to be responsive to treatment with a GLP-1 receptor agonist or analog. In some cases, the assay is used to select the mammal suffering from obesity and/or an obesity-related co-morbidity for treatment with a specific pharmacological intervention. In some cases, if a plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) are detected in a sample obtained from the obese mammal, said obese mammal is selected for treatment with a GLP-1 receptor agonist or analog. In some cases, the method further comprises administering a specific pharmacological intervention based on the detection of the plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929). In some cases, the plurality of SNPs can comprise at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the SNPs selected from the group consisting of rs1664232, rs11118997, rs9342434, rs2335852, rs11020655, rs1885034, rs7277175, rs7903146, rs6923761, rs1047776, rs17782313 and rs3813929. In some cases, the specific pharmacological intervention is a GLP-1 agonist or analog. The GLP-1 agonist can be selected from the group consisting of exenatide, liraglutide, lixisenatide, albiglutide, dulaglutide, tirzepatide and semaglutide. In one embodiment, the GLP-1 receptor analog is liraglutide. In one embodiment, the GLP-1 receptor analog is semaglutide. In some cases, the plurality of SNPs are selected from the group consisting of rs1664232, rs11118997, rs9342434, rs2335852, rs11020655, rs1885034, rs7277175, rs7903146, rs6923761, rs1047776, rs17782313, rs3813929 and any combination thereof. In some cases, the plurality of SNPs comprises, consists essentially of or consists of rs1047776, rs17782313 and rs3813929. In some cases, the plurality of SNPs comprises, consists essentially of or consists of rs11118997, rs1664232, rs6923761, rs9342434, rs2335852, rs11020655 and rs1885034. In some cases, the plurality of SNPs comprises, consists essentially of or consists of rs11118997, rs1664232, rs6923761, rs9342434, rs2335852, rs1885034, rs11020655, rs1047776, rs17782313 and rs3813929. In some cases, the plurality of SNPs comprises, consists essentially of or consists of rs1664232, rs11118997, rs9342434, rs2335852, rs11020655, rs1885034 and rs7277175. In some cases, the plurality of SNPs comprises, consists essentially of or consists of rs1664232, rs11118997, rs9342434, rs2335852, rs11020655, rs1885034, rs7277175, rs7903146 and rs6923761. In some cases, the plurality of SNPs comprises, consists essentially of or consists of rs1664232, rs11118997, rs9342434, rs2335852, rs11020655, rs1885034, rs7277175, rs7903146, rs6923761, rs1047776, rs17782313 and rs3813929.

Provided herein is a system for determining if an obese mammal will respond to treatment with a GLP-1 receptor agonist or for selecting an obese mammal for treatment with a GLP-1 receptor agonist. In one embodiment, the system can comprise: (a) one or more processors; (b) one or more memories operatively coupled to at least one of the one or more processors and having instructions stored thereon that, when executed by at least one of the one or more processors, cause the system to identify the presence or absence of a plurality of SNPs in a sample obtained from a mammal suffering from obesity; and (c) one or more instruments in communication with at least one of the one or more processors, wherein the instruments, upon receipt of instructions sent by the at least one of the one or more processors, perform the identification step. Identification of the plurality of the SNPs in the sample can predict that said obese mammal will respond to treatment with the GLP-1 receptor agonist or select the obese mammal for treatment with a GLP-1 receptor agonist. In some cases, the system further comprises a predictive machine learning model such that the predictive machine learning model is populated with the results of the identification step and the predictive machine learning model uses the identification of the plurality of SNPs to predict responsiveness to or select the obese mammal for treatment with the GLP-1 receptor agonist. The predictive machine learning model can be selected from the group consisting of least absolute shrinkage and selection operator (LASSO) regression, a classification and regression tree (CART) model, and a gradient boosting machine (GBM) model. In one embodiment, the system is further configured such that the one or more memories operatively coupled to the at least one of the one or more processors and having instructions stored thereon that, when executed by at least one of the one or more processors, further cause the system to populate the predictive learning model with data concerning the gastric motor function, resting energy expenditure (REE), one or more measures of appetite, results on behavioral questionnaires or any combination thereof of the subject suffering from obesity. In some cases, the gastric motor function is determined by measuring gastric emptying of the mammal. The gastric emptying can be measured using any method known in the art such as, for example, scintigraphy. In some cases, the REE of the mammal can be measured by indirect calorimetry. In some cases, the one or more measures of appetite can be selected from the group consisting of calories to fullness (CTF), maximum tolerated calories (MTC) and intake calories at an ad libitum buffet meal.

It is intended that the methods and/or systems described herein for determining if an obese mammal will respond to treatment with a GLP-1 receptor agonist or for selecting an obese mammal for treatment with a GLP-1 receptor agonist can be performed by or utilize software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, machine learning models (e.g., LASSO, GBM or CART) and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In one embodiment, provided herein is a system comprising one or more processors, one or more memories, and/or a non-transitory computer readable medium as well as instructions and/or computer code designed to execute any of the diagnostic, prognostic or theranostic methods described herein when executed by at least one of the one or more processors in combination with any hardware devices (e.g., computers, sequencers, microfluidic handling devices) that are specifically configured to store and execute the program code and/or instructions stored in the one or more memories. In some cases, provided herein is a system for determining if an obese mammal will respond to treatment with a GLP-1 receptor agonist or for selecting an obese mammal for treatment with a GLP-1 receptor agonist. In some cases, the results obtained from the system are entered into a database for access by representatives or agents of a business, the individual, a medical provider, or insurance provider. In some cases, the results include sample classification, identification, or diagnosis by a representative, agent or consultant of the obesity phenotyping business, such as a medical professional. In other cases, the system is configured to perform an algorithmic analysis of the results obtained from or by the obese mammal automatically (e.g., through the use of machine learning models such as those provided herein). In some cases, the business may bill the individual, insurance provider, medical provider, researcher, or government entity for one or more of the following: SNP genotyping assays performed, consulting services, data analysis, reporting of results, or database access.

In some embodiments of the present invention, the system is configured such that the results of the SNP analysis are presented as a report on a computer screen or as a paper record. In some embodiments, the report may include, but is not limited to, such information as one or more of the following: the presence/absence of the plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) as compared to the reference sample or reference value(s); the likelihood the subject will respond to a particular intervention (e.g., with a GLP-1 agonist), based on the identification results of the plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929). The reference sample or values can be from a mammal considered to be non-obese or a mammal determined to be obese and to possess the plurality of SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929).

In some cases, the methods and systems for predicting the response of an obese mammal to a GLP-1 agonist or analog, selecting an obese mammal for treatment with a GLP-1 agonist or analog and/or treating said obese mammal with a GLP-1 agonist or analog comprises or further comprises assessing the gastric motor function of the mammal. The assessing the gastric motor function of the mammal can comprise measuring the gastric emptying of the mammal. An increase in or acceleration of gastric emptying for the mammal as compared to gastric emptying in a control can select the mammal for treatment with the GLP-1 agonist. The gastric emptying can be determined or measured prior to treatment (i.e., baseline gastric emptying of the obese mammal) or during treatment. In some cases, an increased or accelerated baseline gastric emptying of an obese mammal as compared to a control alone or in combination with detection of two or more of the aforementioned SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) can select the obese mammal for treatment with a GLP-1 agonist or predict that said obese mammal will respond to GLP-1 agonist treatment. In some cases, delayed gastric emptying of an obese mammal detected during or after treatment (e.g., with a GLP-1 agonist) as compared to a control (e.g., the gastric emptying of the obese mammal prior to treatment) alone or in combination with detection of two or more of the aforementioned SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) can select the obese mammal for further treatment (e.g., with the GLP-1 agonist) or predict that said obese mammal will respond to further treatment with a GLP-1 agonist. The gastric emptying can be measured using any method known in the art such as, for example, scintigraphy. The control can be the rate of gastric emptying in a non-obese mammal, an obese mammal not subject to treatment (e.g., with a GLP-1 agonist), or the rate of gastric emptying in the obese mammal prior to treatment (e.g., with a GLP-1 agonist). The gastric emptying can be GE T1/4 and/or GE T1/4. The gastric emptying can be the GE of solids and/or liquids.

In some cases, the methods and systems for predicting the response of an obese mammal to a GLP-1 agonist or analog, selecting an obese mammal for treatment with a GLP-1 agonist or analog and/or treating said obese mammal with a GLP-1 agonist or analog comprises or further comprises assessing one or more measures of appetite of the mammal. The assessing the one or more measures of appetite of the obese mammal can be selected from the group consisting of calories to fullness (CTF), maximum tolerated calories (MTC) and intake calories at an ad libitum buffet meal. A decrease in one or more measures of appetite for the obese mammal during treatment (e.g., with a GLP-1 agonist) as compared to the same one or more measures in appetite in the obese mammal prior to treatment (e.g., with a GLP-1 agonist) alone or in combination with detection of two or more of the aforementioned SNPs (e.g., two or more SNPs selected from the group consisting of rs1664232, rs11118997, rs9342434, rs2335852, rs11020655, rs1885034, rs7277175, rs7903146, rs6923761, rs1047776, rs17782313 and rs3813929) and/or evidence of a rapid or accelerated gastric emptying prior to or delay in gastric emptying during or after treatment as described herein can select the obese mammal for further treatment with the GLP-1 agonist.

In some cases, the methods and systems provided herein for predicting GLP-1 agonist response in an obese mammal or selecting an obese mammal for treatment with a GLP-1 agonist can further comprise detecting the presence and/or absence of one or more additional SNPs.

Examples of the one or more additional SNPs that can be utilized can comprise coding sequences that a SNP associated with obesity can be in or near and can include, without limitation, the coding sequences selected from the group consisting of transcription elongation regulator 1 like (TCERG1L), pannexin 1 (PANX1), protein tyrosine phosphatase receptor type N2 (PTPRN2), alcohol dehydrogenase 1B (Class I), beta polypeptide (ADH1B), hedgehog acyltransferase (HHAT), lipase C (LIPC), low-density lipoprotein receptor-related protein 1B (LRP1B), retinoic acid receptor beta (RARB), CCR4-NOT transcription complex subunit 2 (CNOT2), fragile histidine triad diadenosine triphosphatase (FHIT), pericentrin (PCNT), adaptor related protein complex 2 subunit beta 1 (AP2B1), regulator of G protein signaling 9 (RGS9), chromosome 8 open reading frame 37 (C8ORF37), receptor tyrosine-protein kinase erbB-4 (ERBB4), parkin RBR E3 ubiquitin protein ligase (PRKN), neurotrophic receptor tyrosine kinase 2 (NTRK2), eyes shut homolog (EYS), Parkinson disease 2 (PARK2), FERM domain containing 6 (FRMD6), plexin A1 (PLXNA1), glycosyltransferase 1 domain containing 1 (GLTID1), transcription factor 7 like 2 (TCF7L2), glucagon-like peptide 1 receptor (GLP1R), melanocortin 4 receptor (MC4R), SIM BHLH transcription factor 1 (SIM1), and brain-derived neurotrophic factor (BDNF), 5-hydroxytryptamine (serotonin) receptor 2C (HTR2C), ADRA2A, ADRA2C, GNB3, FTO, 5-HTTLPR, UCP2, UCP3, GPBAR1, NR1H4, FGFR4, PYY, GLP-1, CCK, leptin, adiponectin, neurotensin, ghrelin, GLP-1 receptor, GOAT, DPP4, POMC, NPY, AGRP, SERT, SLC6A4, DRD2, LEP, LEPR, UCP1, KLF14, NPC1, LYPLAL1, ADRB2, ADRB3, BBS1, ACSL6, ADARB2, ADCY8, AJAP1, ATP2C2, ATP6V0D2, C21orf7, CAMKMT, CAP2, CASC4, CD48, CDC42SE2, CDYL, CES5AP1, CLMN, CNPY4, COL19A1, COL27A1, COL4A3, CORO1C, CPZ, CTIF, DAAM2, DCHS2, DOCKS, EGFLAM, FAM125B, FAM71E2, FRMD3, GALNTL4, KRT23, LHPP, LINC00578, LINC00620, LOC100128714, LOC100287160, LOC100289473, LOC100293612|LINC00620, LOC100506869, LOC100507053, LOC100507053|ADHIA, LOC100507053|ADH, LOC100507443, LOC100996571|CYYR1, LOC152225, LOC255130, LPAR1, LUZP2, MCM7, MICAL3, MMS19, MYBPC1, NR2F2-AS1, NSMCE2, NTN1, O3FAR1, OAZ2, OSBP2, P4HA2, PADI1, PARD3B, PCDH15, PIEZO2, PKIB, PRH1-PRR4, PTPRD, RALGPS1|ANGPTL2, RPS24P10, RTN4RL1, RYR2, SCN2A, SEMA3C, SEMA5A, SFMBT2, SGCG, SLC22A15, SLC2A2, SLCO1B1, SMOC2,SNCAIP, SNX18, SRRM4, SUSD1, TBC1D16, TENM3, TJP3, TLL1, TMEM9B, TPM1, VTI1A, VWF, WWOX, WWTR1, ZFYVE28, ZNF3, ZNF609, and ZSCAN21. Examples of the one or more additional SNPs can include, without limitation, rs657452, rs11583200, rs2820292, rs11126666, rs11688816, rs1528435, rs7599312, rs6804842, rs2365389, rs3849570, rs16851483, rs17001654, rs11727676, rs2033529, rs9400239, rs13191362, rs1167827, rs2245368, rs2033732, rs4740619, rs6477694, rs1928295, rs10733682, rs7899106, rs17094222, rs11191560, rs7903146, rs2176598, rs12286929, rs11057405, rs10132280, rs12885454, rs3736485, rs758747, rs2650492, rs9925964, rs1000940, rs1808579, rs7243357, rs17724992, rs977747, rs1460676, rs17203016, rs13201877, rs1441264, rs7164727, rs2080454, rs9914578, rs2836754, rs492400, rs16907751, rs9374842, rs9641123, rs9540493, rs4787491, rs6465468, rs7239883, rs3101336, rs12566985, rs12401738, rs11165643, rs17024393, rs543874, rs13021737, rs10182181, rs1016287, rs2121279, rs13078960, rs1516725, rs10938397, rs13107325, rs2112347, rs205262, rs2207139, rs17405819, rs10968576, rs4256980, rs11030104, rs3817334, rs7138803, rs12016871, rs12429545, rs11847697, rs7141420, rs16951275, rs12446632, rs3888190, rs1558902, rs12940622, rs6567160, rs29941, rs2075650, rs2287019, rs3810291, rs7715256, rs2176040, rs6091540, rs1800544, Ins-Del-322, rs5443, rs1129649, rs1047776, rs9939609, rs17782313, rs7903146, rs4795541, rs3813929, rs518147, rs1414334, rs659366, −3474, rs2075577, rs15763, rs1626521, rs11554825, rs4764980, rs434434, rs351855, and rs2234888.

In some cases, the methods and systems provided herein for predicting GLP-1 agonist response in an obese mammal or selecting an obese mammal for treatment with a GLP-1 agonist can further comprise the obese mammal filling out or completing one or more questionnaires. In some cases, the behavioral questionnaire can be any questionnaire associated with obesity. The behavioral questionnaire can be psychological welfare questionnaires, alcohol use questionnaires, eating behavior questionnaires, body image questionnaires, physical activity level questionnaire, and weight management questionnaires. Examples of questionnaires that can include, without limitation, The Hospital Anxiety and Depression Scale (HADS) questionnaire, The Hospital Anxiety and Depression Inventory questionnaire, The Questionnaire on Eating and Weight Patterns, The Weight Efficacy Life-Style (WEL) Questionnaire, Three-Factor Eating Questionnaire (TFEQ), and The Multidimensional Body-Self Relations Questionnaire. For example, a questionnaire can be a HADS questionnaire.

The methods and systems provided herein for predicting GLP-1 agonist response in an obese mammal or selecting an obese mammal for treatment with a GLP-1 agonist can do so with a sensitivity and/or specificity of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein for predicting GLP-1 agonist response in an obese mammal or selecting an obese mammal for treatment with a GLP-1 agonist can do so with a predictive success (e.g., positive predictive value (PPV) or negative predictive value (NPV)) of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein for predicting GLP-1 agonist response in an obese mammal or selecting an obese mammal for treatment with a GLP-1 agonist can do so with a precision of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein for predicting GLP-1 agonist response in an obese mammal or selecting an obese mammal for treatment with a GLP-1 agonist can do so with an accuracy of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

### Multi-Omic/Machine Learning Based Models for Determining Obesity Phenotype

Provided herein are methods and systems for identifying or determining the obesity phenotype of mammal suffering from obesity. In some cases, the method and systems provided herein for identifying or determining the obesity phenotype of the mammal suffering from obesity utilizes or employs a machine learning model. The machine learning model can be selected from the group consisting of least absolute shrinkage and selection operator (LASSO) regression, a classification and regression tree (CART) model, and a gradient boosting machine (GBM) model. The machine learning models used in the methods and systems provided herein can incorporate data related to the obese mammal selected from the group consisting of metabolomics, genomics, microbiome, proteomic, peptidomics, and behavioral questionnaires. In some cases, the data specific to the obese mammal that can be utilized by the machine learning models can include, but not be limited to, demographic information, genome-wide association study (GWAS) results, metabolomic results, behavioral questionnaire results, the detected presence and/or absence of gastrointestinal peptides or hormones, the detected presence and/or absence of metabolites, the detected presence and/or absence of genetic variants, assessment of gastric motor functions and assessment of appetite. In some cases, the methods and systems provided herein further provide for selecting and/or administering a pharmacological intervention for treating the obesity in the mammal based on the determined obesity phenotype. In some cases, the methods and systems provided herein can be used to determine if a mammal suffering from obesity is likely to be responsive to an intervention (e.g., pharmacological intervention) based, at least in part, on an obesity phenotype, which is based, at least in part, on an analyte signature determined for a sample obtained from the mammal. The obesity phenotypes that can be determined using the methods and systems provided herein can be selected from the group consisting of hungry brain (e.g., abnormal satiation), hungry gut (e.g., abnormal satiety), emotional hunger (e.g., abnormal hedonic eating), slow burn (e.g., abnormal metabolism), and mixed. In some cases, the obesity phenotypes that are determined using the methods and systems provided herein are selected from the group consisting of hungry brain (e.g., abnormal satiation), hungry gut (e.g., abnormal satiety), emotional hunger (e.g., abnormal hedonic eating) and slow burn (e.g., abnormal metabolism). In some cases, each obesity phenotype is likely to be responsive to one or more particular interventions as provided herein. The obesity analyte signature in sample obtained from an obese mammal (and thus the obesity phenotype) can be used to predict intervention responsiveness. The one or more interventions can be selected from the group consisting of pharmacological intervention, surgical intervention, weight loss device, diet intervention, behavior intervention, and microbiome intervention. For example, a sample obtained from the mammal can be assessed for pharmacological intervention responsiveness using the methods and/or systems provided herein.

In one embodiment, provided herein is a system for determining an obesity phenotype of a mammal suffering from obesity, the system comprising: (a) one or more processors; (b) one or more memories operatively coupled to at least one of the one or more processors and (c) one or more instruments in communication with at least one of the one or more processors, In some cases, the one or more memories operatively coupled to at least one of the one or more processors have instructions stored thereon that, when executed by at least one of the one or more processors, cause the system to (i) identify the presence, absence or level of a plurality of gastrointestinal (GI) peptides, a plurality of metabolites, and/or a plurality of genetic variants in a sample obtained from a mammal suffering from obesity, thereby generating an analyte signature for the sample; (ii) populate a predictive machine learning model with the analyte signature of step (i); and (iii) utilize the predictive machine learning model to predict an obesity phenotype of the mammal suffering from obesity based on the analyte signature of the sample, wherein the obesity phenotype is selected from the group consisting of. In some cases, the one or more instruments in communication with the at least one of the one or more processors, wherein the one or more instruments, upon receipt of instructions sent by the at least one of the one or more processors, perform steps (i)-(iii). In some cases, the predictive machine learning model is selected from the group consisting of least absolute shrinkage and selection operator (LASSO) regression, a classification and regression tree (CART) model, and a gradient boosting machine (GBM) model.

In another embodiment, provided herein is a method for treating obesity in a mammal, the method comprising: (a) identifying the presence, absence or level of a plurality of GI peptides, a plurality of metabolites, and/or a plurality of genetic variants in a sample obtained from a mammal suffering from obesity, thereby generating an analyte signature for the sample; (b) populating a predictive machine learning model with the analyte signature of step (a); (c) utilizing the predictive machine learning model to predict an obesity phenotype of the mammal based on the analyte signature of the sample obtained from the mammal, wherein the obesity phenotype is selected from the group consisting of abnormal satiation (hungry brain), abnormal satiety (hungry gut); hedonic eating (emotional hunger) and slow metabolism (slow burn); and (d) administering an intervention based on the obesity phenotype predicted in step (c). In some cases, the predictive machine learning model is selected from the group consisting of least absolute shrinkage and selection operator (LASSO) regression, a classification and regression tree (CART) model, and a gradient boosting machine (GBM) model.

It is intended that the methods and/or systems described herein can be performed by or utilize software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, machine learning models (e.g., GBM or CART) and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In one embodiment, provided herein is a system comprising one or more processors, one or more memories, and/or a non-transitory computer readable medium as well as instructions and/or computer code designed to execute any of the diagnostic, prognostic or theranostic methods described herein when executed by at least one of the one or more processors in combination with any hardware devices (e.g., computers, sequencers, microfluidic handling devices) that are specifically configured to store and execute the program code and/or instructions stored in the one or more memories. In some cases, provided herein is a system for determining an obesity phenotype or analyte signature of a sample obtained from a subject suffering from obesity. The system can be used to diagnose or determine the obesity phenotype of the subject based on the integration and analysis of metabolomic, genomic, microbiome, proteomic, peptidomic, and/or behavioral questionnaire results utilizing machine learning models. The system may also be used to predict responsive of the mammal to a particular intervention as provided herein as a result of determining the mammal's obesity phenotype. In some cases, the system comprises one or more processors and one or more memories operatively coupled to at least one of the one or more processors and having instructions stored thereon that, when executed by at least one of the one or more processors, cause the system to perform or integrate the results of metabolomic, genomic, microbiome, proteomic, peptidomic, and/or behavioral questionnaire conduct on or by the obese mammal. In some cases, the results of the metabolomic, genomic, microbiome, proteomic, peptidomic, and/or behavioral questionnaire results obtained from or by the obese mammal are entered into a database for access by representatives or agents of a business, the individual, a medical provider, or insurance provider. In some cases, assay results include sample classification, identification, or diagnosis by a representative, agent or consultant of the obesity phenotyping business, such as a medical professional. In other cases, the system is configured to perform an algorithmic analysis of the metabolomic, genomic, microbiome, proteomic, peptidomic, and/or behavioral questionnaire results obtained from or by the obese mammal automatically (e.g., through the use of machine learning models such as those provided herein). In some cases, the business may bill the individual, insurance provider, medical provider, researcher, or government entity for one or more of the following: obesity phenotyping assays performed, consulting services, data analysis, reporting of results, or database access.

In some embodiments of the present invention, the system is configured such that the results of the obesity phenotyping assays are presented as a report on a computer screen or as a paper record. In some embodiments, the report may include, but is not limited to, such information as one or more of the following: the presence/absence/levels of biomarkers as compared to the reference sample or reference value(s); the likelihood the subject will respond to a particular intervention, based on the obesity phenotype and/or analyte signature and the obesity phenotype and proposed therapies. The reference sample or values can be from a mammal considered to be non-obese or a mammal determined to be obese and to possess one or more biomarkers associated with a specific obesity phenotype (e.g., abnormal satiation, abnormal satiety, emotional hunger or slow burn). In some cases, the reference sample can be a plurality of reference samples, wherein the plurality comprises samples from obese mammals determined to possess a biomarker or analyte signature associated with each of the specific obesity phenotypes described herein (e.g., abnormal satiation, abnormal satiety, emotional hunger or slow burn). In some cases, the reference values can be a plurality of reference samples, wherein the plurality of reference samples comprise samples from obese mammals determined to possess a biomarker or analyte signature associated with each of the specific obesity phenotypes described herein (e.g., abnormal satiation, abnormal satiety, emotional hunger or slow burn) and the reference values can represent analyte signatures associated with each specific obesity phenotype provided herein (e.g., abnormal satiation, abnormal satiety, emotional hunger or slow burn).

An analyte signature for use in the methods and/or systems provided herein for determining an obese mammal's obesity phenotype can include the presence, absence, or level (e.g., concentration) of one or more (e.g., two, three, four, five, six, seven, eight, nine, 10, 11, 12, 13, 14, 15, 16, 17, 18, 19, 20, or more) obesity analytes (e.g., biomarkers associated with obesity). The obesity analytes can be gastrointestinal (GI) hormones/peptides, genetic variants in specific genes and one or more metabolites.

The GI peptides or hormones that can be utilized by the machine learning models utilized in the methods and systems provided herein can include any gastrointestinal peptide that is associated with obesity. In some cases, a gastrointestinal peptide can be a peptide hormone. In some cases, a gastrointestinal peptide can be released from gastrointestinal cells in response to feeding. In some cases, a gastrointestinal peptide can be any GI peptide described in WO2019104146A1, which is herein incorporated by reference in its entirety. Examples of gastrointestinal peptides that can be used to determine the obesity analyte signature in a sample (e.g., in a sample obtained from an obese mammal) include, without limitation, ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide.

The genetic variants that can be utilized by the machine learning models utilized in the methods and systems provided herein can include detecting the presence or absence of a single nucleotide polymorphism (SNP). The SNP can be any SNP that is associated with obesity. The SNP can be any SNP provided herein (e.g., SNPs described in Table 3 (i.e., rs1664232, rs11118997, rs9342434, rs2335852, rs11020655, rs1885034, rs7277175) and/or rs6923761, rs7903146, rs1047776, rs17782313 and rs3813929) alone or in combination with one or more SNPs known in the prior art to associated with obesity such as, for example, the SNPs described as being associated with obesity in WO2019104146A1, which is herein incorporated by reference in its entirety for all purposes. A SNP can be in a coding sequence (e.g., in a gene) or a non-coding sequence. For example, in cases where a SNP is in a coding sequence, the coding sequence can be any appropriate coding sequence.

In some cases, a coding sequence that can include a SNP associated with obesity can be in a gene shown in Table 3. Examples of coding sequences that a SNP associated with obesity can be in or near include, without limitation, the coding sequences selected from the group consisting of transcription elongation regulator 1 like (TCERG1L), pannexin 1 (PANX1), protein tyrosine phosphatase receptor type N2 (PTPRN2), alcohol dehydrogenase 1B (Class I), beta polypeptide (ADH1B), hedgehog acyltransferase (HHAT), lipase C (LIPC), low-density lipoprotein receptor-related protein 1B (LRP1B), retinoic acid receptor beta (RARB), CCR4-NOT transcription complex subunit 2 (CNOT2), fragile histidine triad diadenosine triphosphatase (FHIT), pericentrin (PCNT), adaptor related protein complex 2 subunit beta 1 (AP2B1), regulator of G protein signaling 9 (RGS9), chromosome 8 open reading frame 37 (C80RF37), receptor tyrosine-protein kinase erbB-4 (ERBB+), parkin RBR E3 ubiquitin protein ligase (PRKN), neurotrophic receptor tyrosine kinase 2 (NTRK2), eyes shut homolog (EYS), Parkinson disease 2 (PARK2), FERM domain containing 6 (FRMD6), plexin A1 (PLXNA1), glycosyltransferase 1 domain containing 1 (GLTID1), transcription factor 7 like 2 (TCF71.2), glucagon-like peptide 1 receptor (GLP1R), melanocortin 4 receptor (MC4R), SIM BHLH transcription factor 1 (SIM1), and brain-derived neurotrophic factor (BDNF), 5-hydroxytryptamine (serotonin) receptor 2C (HTR2C), ADRA2A, ADRA2C, GNB3, FTO, 5-HTTLPR, UCP2, UCP3, GPBAR1, NR1H4, FGFR4, PYY, GLP-1, CCK, leptin, adiponectin, neurotensin, ghrelin, GLP-1 receptor, GOAT, DPP4, POMC, NPY, AGRP, SERT, SLC6A4, DRD2, LEP, LEPR, UCP1, KLF14, NPC1, LYPLAL1, ADRB2, ADRB3, BBS1, ACSL6, ADARB2, ADCY8, AJAP1, ATP2C2, ATP6V0D2, C21orf7, CAMKMT, CAP2, CASC4, CD48, CDC42SE2, CDYL, CES5AP1, CLMN, CNPY4, COL19A1, COL27A1, COL4A3, CORO1C, CPZ, CTIF, DAAM2, DCHS2, DOCKS, EGFLAM, FAM125B, FAM71E2, FRMD3, GALNTL4, KRT23, LHPP, LINC00578, LINC00620, LOC100128714, LOC100287160, LOC100289473, LOC100293612|LINC00620, LOC100506869, LOC100507053, LOC100507053|ADHIA, LOC100507053|ADH, LOC100507443, LOC100996571|CYYR1, LOC152225, LOC255130, LPAR1, LUZP2, MCM7, MICAL3, MMS19, MYBPC1, NR2F2-AS1, NSMCE2, NTN1, O3FAR1, OAZ2, OSBP2, P4HA2, PADI1, PARD3B, PCDH15, PIEZO2, PKIB, PRH1-PRR4, PTPRD, RALGPS1|ANGPTL2, RPS24P10, RTN4RL1, RYR2, SCN2A, SEMA3C, SEMA5A, SFMBT2, SGCG, SLC22A15, SLC2A2, SLCO1B1, SMOC2,SNCAIP, SNX18, SRRM4, SUSD1, TBC1D16, TENM3, TJP3, TLL1, TMEM9B, TPM1, VTI1A, VWF, WWOX, WWTR1, ZFYVE28, ZNF3, ZNF609, and ZSCAN21.

In some cases, a SNP for use in the methods and system provided herein comprises, consists essentially of or consists of a SNP shown in Table 3. In some cases, a SNP for use in the methods and system provided herein comprises, consists essentially of or consists of rs1664232, rs11118997, rs9342434, rs2335852, rs11020655, rs1885034, rs7277175, rs7903146, and rs6923761. In some cases, a SNP for use in the methods and system provided herein comprises, consists essentially of or consists of rs1664232, rs11118997, rs9342434, rs2335852, rs11020655, rs1885034, rs7277175, rs1047776, rs17782313 and rs3813929. In some cases, a SNP for use in the methods and system provided herein comprises, consists essentially of or consists of rs1664232, rs11118997, rs9342434, rs2335852, rs1885034, rs7903146, rs6923761, rs1047776, rs17782313 and rs3813929. In some cases, a SNP for use in the methods and system provided herein comprises, consists essentially of or consists of rs1047776, rs17782313 and rs3813929. In some cases, one or more additional SNPs are detected in a system or method provided herein. Examples of additional SNPs can include, without limitation, rs657452, rs11583200, rs2820292, rs11126666, rs11688816, rs1528435, rs7599312, rs6804842, rs2365389, rs3849570, rs16851483, rs17001654, rs11727676, rs2033529, rs9400239, rs13191362, rs1167827, rs2245368, rs2033732, rs4740619, rs6477694, rs1928295, rs10733682, rs7899106, rs17094222, rs11191560, rs7903146, rs2176598, rs12286929, rs11057405, rs10132280, rs12885454, rs3736485, rs758747, rs2650492, rs9925964, rs1000940, rs1808579, rs7243357, rs17724992, rs977747, rs1460676, rs17203016, rs13201877, rs1441264, rs7164727, rs2080454, rs9914578, rs2836754, rs492400, rs16907751, rs9374842, rs9641123, rs9540493, rs4787491, rs6465468, rs7239883, rs3101336, rs12566985, rs12401738, rs11165643, rs17024393, rs543874, rs13021737, rs10182181, rs1016287, rs2121279, rs13078960, rs1516725, rs10938397, rs13107325, rs2112347, rs205262, rs2207139, rs17405819, rs10968576, rs4256980, rs11030104, rs3817334, rs7138803, rs12016871, rs12429545, rs11847697, rs7141420, rs16951275, rs12446632, rs3888190, rs1558902, rs12940622, rs6567160, rs29941, rs2075650, rs2287019, rs3810291, rs7715256, rs2176040, rs6091540, rs1800544, Ins-Del-322, rs5443, rs1129649, rs1047776, rs9939609, rs17782313, rs7903146, rs4795541, rs3813929, rs518147, rs1414334, rs659366, −3474, rs2075577, rs15763, rs1626521, rs11554825, rs4764980, rs434434, rs351855, and rs2234888.

The metabolites that can be utilized by the machine learning models utilized in the methods and systems provided herein can include any metabolite that is associated with obesity. In some cases, a metabolite can be an amino-compound. In some cases, a metabolite can be a neurotransmitter. In some cases, a metabolite can be a fatty acid (e.g., a short chain fatty acid). In some cases, a metabolite can be an amino compound. In some cases, a metabolite can be a bile acid. Examples of metabolites that can be used to determine the obesity analyte signature in a sample (e.g., in a sample obtained from an obese mammal) include, without limitation, 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

In one embodiment, the systems provided herein are further configured such that the one or more memories operatively coupled to the at least one of the one or more processors and having instructions stored thereon that, when executed by at least one of the one or more processors, further cause the system to populate the predictive learning model with data concerning the gastric motor function, resting energy expenditure (REE), one or more measures of appetite, results on behavioral questionnaires or any combination thereof of the subject suffering from obesity.

In one embodiment, the methods provided herein for determining the obesity phenotype further comprise populating the predictive learning model with data concerning the gastric motor function, resting energy expenditure (REE), one or more measures of appetite, results on behavioral questionnaires or any combination thereof of the mammal suffering from obesity.

In some cases, the gastric motor function is determined by measuring gastric emptying of the mammal. The gastric emptying can be measured using any method known in the art such as, for example, scintigraphy. In some cases, the REE of the mammal can be measured by indirect calorimetry.

In some cases, the one or more measures of appetite can be selected from the group consisting of calories to fullness (CTF), maximum tolerated calories (MTC) and intake calories at an ad libitum buffet meal.

In some cases, the behavioral questionnaire can be any questionnaire associated with obesity. The behavioral questionnaire can be psychological welfare questionnaires, alcohol use questionnaires, eating behavior questionnaires, body image questionnaires, physical activity level questionnaire, and weight management questionnaires. Examples of questionnaires that can be used to determine the obesity phenotype of a mammal (e.g., an obese mammal) include, without limitation, The Hospital Anxiety and Depression Scale (HADS) questionnaire, The Hospital Anxiety and Depression Inventory questionnaire, The Questionnaire on Eating and Weight Patterns, The Weight Efficacy Life-Style (WEL) Questionnaire, Three-Factor Eating Questionnaire (TFEQ), and The Multidimensional Body-Self Relations Questionnaire. For example, a questionnaire can be a HADS questionnaire.

The methods and systems provided herein can determine, identify or predict an obesity phenotype of the mammal suffering from obesity with a sensitivity and/or specificity of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein can determine, identify or predict an obesity phenotype of the mammal suffering from obesity with a predictive success (e.g., positive predictive value (PPV) or negative predictive value (NPV)) of at least about 60%, at least about 61%, at least about 62%, at least about 63%, at least about 64%, at least about 65%, at least a bout 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein can determine, identify or predict an obesity phenotype of the mammal suffering from obesity with a precision of at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The methods and systems provided herein can determine, identify or predict an obesity phenotype of the mammal suffering from obesity with an accuracy of at least about 60%, at least about 61%, at least about 62%, at least about 63%, at least about 64%, at least about 65%, at least a bout 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

As described previously herein, once the obesity phenotype of the mammal has been identified, the obesity phenotype can be used to select a treatment option for the mammal. For example, once a mammal is identified as being responsive to one or more interventions (e.g., pharmacological intervention, surgical intervention, weight loss device, diet intervention, behavior intervention, and/or microbiome intervention) based, at least in part, on an obesity phenotype, which is based, at least in part, on an obesity analyte signature in the sample, the mammal can be administered or instructed to self-administer one or more pharmacological interventions.

Individualized pharmacological interventions for the treatment of obesity (e.g., based on the obesity phenotypes as determined using the methods and/or system provided herein) can include any one or more (e.g., 1, 2, 3, 4, 5, 6, or more) pharmacotherapies (e.g., individualized pharmacotherapies). A pharmacotherapy can include any appropriate pharmacotherapy. In some cases, a pharmacotherapy can be an obesity pharmacotherapy. In some cases, a pharmacotherapy can be an appetite suppressant. In some cases, a pharmacotherapy can be an anticonvulsant. In some cases, a pharmacotherapy can be a GLP-1 agonist. In some cases, a pharmacotherapy can be an antidepressant. In some cases, a pharmacotherapy can be an opioid antagonist. In some cases, a pharmacotherapy can be a controlled release pharmacotherapy. For example, a controlled release pharmacotherapy can be an extended release (ER) and/or a slow release (SR) pharmacotherapy. In some cases, a pharmacotherapy can be a lipase inhibitor. In some cases, a pharmacotherapy can be a DPP4 inhibitor. In some cases, a pharmacotherapy can be a SGLT2 inhibitor. In some cases, a pharmacotherapy can be a dietary supplement. Examples of pharmacotherapies that can be used in an individualized pharmacological intervention as described herein include, without limitation, orlistat, phentermine, topiramate, lorcaserin, naltrexone, bupropion, liraglutide, semaglutide, albiglutide, dulaglutide, lixisenatide, exenatide, metformin, pramlitide, Januvia, canagliflozin, dexamphetamines, prebiotics, probiotics, Ginkgo biloba, and combinations thereof. For example, combination pharmacological interventions for the treatment of obesity (e.g., based on the obesity phenotypes determined using the methods and/or system provided herein) can include phentermine-topiramate ER, naltrexone-bupropion SR, phentermine-lorcaserin, lorcaserin-liraglutide, and lorcarserin-januvia. In some cases, a pharmacotherapy can be administered as described elsewhere (see, e.g., Sjostrom et al., 1998 Lancet 352:167-72; Hollander et al., 1998 Diabetes Care 21:1288-94; Davidson et al., 1999 JAMA 281:235-42; Gadde et al., 2011 Lancet 377:1341-52; Smith et al., 2010 New Engl. J. Med. 363:245-256; Apovian et al., 2013 Obesity 21:935-43; Pi-Sunyer et al., 2015 New Engl. J. Med. 373:11-22; and Acosta et al., 2015 Clin Gastroenterol Hepatol. 13:2312-9).

In some cases, when a mammal is identified as having a hungry gut (e.g., abnormal satiety) phenotype as determined using the methods and/or system provided herein, the mammal can be administered or instructed to self-administer one or more GLP-1 agonists (e.g., liraglutide) to treat the obesity. The GLP-1 agonist can be selected from the group consisting of liraglutide, semaglutide, albiglutide, dulaglutide, tirzepatide, lixisenatide and exenatide.

In some cases, when a mammal is identified as having a hungry brain (e.g., abnormal satiation) phenotype as determined using the methods and/or system provided herein, the mammal can be administered or instructed to self-administer phentermine, topiramate, lorcaserin and any combination thereof to treat the obesity. In some cases, when a mammal is identified as having a hungry brain (e.g., abnormal satiation) phenotype as determined using the methods and/or system provided herein, the mammal is administered or instructed to self-administer phentermine-topiramate.

In some cases, when a mammal is identified as having a hedonic eating (emotional hunger) phenotype as determined using the methods and/or system provided herein, the mammal can be administered or instructed to self-administer naltrexone-bupropion pharmacotherapy.

In some cases, when a mammal is identified as having a slow metabolism (e.g., slow burn) phenotype as determined using the methods and/or system provided herein, the mammal can be administered or instructed to self-administer phentermine, topiramate, lorcaserin and any combination thereof to treat the obesity. In some cases, when a mammal is identified as having a slow metabolism (e.g., slow burn) phenotype as determined using the methods and/or system provided herein, the mammal is administered or instructed to self-administer phentermine pharmacotherapy.

In some cases, one or more pharmacotherapies described herein can be administered to an obese mammal as a combination therapy with one or more additional agents/therapies used to treat obesity. For example, a combination therapy used to treat an obese mammal (e.g., an obese human) can include administering to the mammal one or more pharmacotherapies described herein and one or more obesity treatments such as weight-loss surgeries (e.g., gastric bypass surgery, laparoscopic adjustable gastric banding (LAGB), biliopancreatic diversion with duodenal switch, and a gastric sleeve), vagal nerve blockade, endoscopic devices (e.g., intragastric balloons or endoliners, magnets), endoscopic sleeve gastroplasty, and/or gastric or duodenal ablations. For example, a combination therapy used to treat an obese mammal (e.g., an obese human) can include administering to the mammal one or more pharmacotherapies described herein and one or more obesity therapies such as exercise modifications (e.g., increased physical activity), dietary modifications (e.g., reduced-calorie diet), behavioral modifications, commercial weight loss programs, wellness programs, and/or wellness devices (e.g. dietary tracking devices and/or physical activity tracking devices). In cases where one or more pharmacotherapies described herein are used in combination with one or more additional agents/therapies used to treat obesity, the one or more additional agents/therapies used to treat obesity can be administered/performed at the same time or independently. For example, the one or more pharmacotherapies described herein can be administered first, and the one or more additional agents/therapies used to treat obesity can be administered/performed second, or vice versa.

### Co-Morbidities

When treating obesity in a mammal (e.g., a human) as a result of use of the methods and/or systems provided herein, the mammal can have one or more weight-related co-morbidities. Examples of weight-related co-morbidities include, without limitation, hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis, and atherosclerosis (coronary artery disease and/or cerebrovascular disease). In some cases, the methods and materials described herein can be used to treat the one or more weight-related co-morbidities.

### Clinical Uses

When treating obesity in a mammal (e.g., a human) as a result of use of the methods and/or systems provided herein, the treatment can be effective to reduce the weight, reduce the waist circumference, reduce the percentage of fat and/or slow or prevent weight gain of the mammal. For example, the treatment described herein can be effective to reduce the weight (e.g., the total body weight) of an obese mammal by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. Treatment described herein can be effective to reduce the weight (e.g., the total body weight) of an obese mammal by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. Treatment described herein can be effective to reduce the weight (e.g., the total body weight) of an obese mammal by at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. In some cases, the treatment described herein can be effective to reduce the weight (e.g., the total body weight) of an obese mammal by at least 3%, at least 5%, at least 8%, at least 10%, at least 12%, at least 15%, at least 18%, at least 20%, at least 22%, at least 25%, at least 28%, at least 30%, at least 33%, at least 36%, at least 39%, or at least 40%). For example, the treatment described herein can be effective to reduce the weight (e.g., the total body weight) of an obese mammal by from about 3% to about 40% (e.g., from about 3% to about 35%, from about 3% to about 30%, from about 3% to about 25%, from about 3% to about 20%, from about 3% to about 15%, from about 3% to about 10%, from about 3% to about 5%, from about 5% to about 40%, from about 10% to about 40%, from about 15% to about 40%, from about 20% to about 40%, from about 25% to about 40%, from about 35% to about 40%, from about 5% to about 35%, from about 10% to about 30%, from about 15% to about 25%, or from about 18% to about 22%). For example, the treatment described herein can be effective to reduce the weight (e.g., the total body weight) of an obese mammal by from about 3 kg to about 100 kg (e.g., about 5 kg to about 100 kg, about 8 kg to about 100 kg, about 10 kg to about 100 kg, about 15 kg to about 100 kg, about 20 kg to about 100 kg, about 30 kg to about 100 kg, about 40 kg to about 100 kg, about 50 kg to about 100 kg, about 60 kg to about 100 kg, about 70 kg to about 100 kg, about 80 kg to about 100 kg, about 90 kg to about 100 kg, about 3 kg to about 90 kg, about 3 kg to about 80 kg, about 3 kg to about 70 kg, about 3 kg to about 60 kg, about 3 kg to about 50 kg, about 3 kg to about 40 kg, about 3 kg to about 30 kg, about 3 kg to about 20 kg, about 3 kg to about 10 kg, about 5 kg to about 90 kg, about 10 kg to about 75 kg, about 15 kg to about 50 kg, about 20 kg to about 40 kg, or about 25 kg to about 30 kg). For example, the treatment described herein can be effective to reduce the waist circumference of an obese mammal by from about 1 inches to about 10 inches (e.g., about 1 inches to about 9 inches, about 1 inches to about 8 inches, about 1 inches to about 7 inches, about 1 inches to about 6 inches, about 1 inches to about 5 inches, about 1 inches to about 4 inches, about 1 inches to about 3 inches, about 1 inches to about 2 inches, about 2 inches to about 10 inches, about 3 inches to about 10 inches, about 4 inches to about 10 inches, about 5 inches to about 10 inches, about 6 inches to about 10 inches, about 7 inches to about 10 inches, about 8 inches to about 10 inches, about 9 inches to about 10 inches, about 2 inches to about 9 inches, about 3 inches to about 8 inches, about 4 inches to about 7 inches, or about 5 inches to about 7 inches). For example, the treatment described herein can be effective to reduce the fat mass or body fat of an obese mammal by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. The treatment described herein can be effective to reduce the fat mass or body fat of an obese mammal by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. The treatment described herein can be effective to reduce the fat mass or body fat of an obese mammal by at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%.

In some cases, the treatment described herein can be effective to delay or decrease the gastric emptying rate of an obese mammal as compared to the gastric emptying rate of the same obese mammal prior to the treatment by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. In some cases, the treatment described herein can be effective to delay or decrease the gastric emptying rate of an obese mammal as compared to the gastric emptying rate of the same obese mammal prior to the treatment by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%. In some cases, the treatment described herein can be effective to delay or decrease the gastric emptying rate of an obese mammal as compared to the gastric emptying rate of the same obese mammal prior to the treatment by at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% or 45%.

### Subjects

Any type of mammal can be assessed and/or treated using the methods and/or systems provided herein. Examples of mammals that can be assessed and/or treated as described herein include, without limitation, primates (e.g., humans and monkeys), dogs, cats, horses, cows, pigs, sheep, rabbits, mice, and rats. In some cases, the mammal can be a human. In some cases, a mammal can be an obese mammal. For example, obese humans can be assessed for intervention (e.g., a pharmacological intervention) responsiveness, and treated with one or more interventions as described herein.

Any appropriate method can be used to identify a mammal as being obese. In some cases, calculating body mass index (BMI), measuring waist and/or hip circumference, health history (e.g., weight history, weight-loss efforts, exercise habits, eating patterns, other medical conditions, medications, stress levels, and/or family health history), physical examination (e.g., measuring your height, checking vital signs such as heart rate blood pressure, listening to your heart and lungs, and examining your abdomen), percentage of body fat and distribution, percentage of visceral and organs fat, metabolic syndrome, and/or obesity related comorbidities can be used to identify mammals (e.g., humans) as being obese. For example, a BMI of greater than about 30 kg/m² can be used to identify mammals as being obese. For example, a BMI of greater than about 27 kg/m² with a co-morbidity can be used to identify mammals as being obese.

### Sample Types

Any appropriate sample from a mammal (e.g., a human) having obesity can be assessed as described herein. In some cases, a sample can be a biological sample. In some cases, a sample can contain obesity analytes (e.g., DNA, RNA, proteins, peptides, metabolites, hormones, and/or exogenous compounds (e.g., medications)). Examples of samples that can be assessed as described herein include, without limitation, fluid samples (e.g., blood, serum, plasma, urine, saliva, or tears), breath samples, cellular samples (e.g., buccal samples), tissue samples (e.g., adipose samples), stool samples, gastro samples, and intestinal mucosa samples. In some cases, a sample (e.g., a blood sample) can be collected while the mammal is fasting (e.g., a fasting sample such as a fasting blood sample). In some cases, a sample can be processed (e.g., to extract and/or isolate obesity analytes). For example, a serum sample can be obtained from an obese mammal and can be assessed to determine if the obese mammal is likely to be responsive to one or more interventions (e.g., pharmacological intervention, surgical intervention, weight loss device, diet intervention, behavior intervention, and/or microbiome intervention) based, at least in part, on an obesity phenotype, which is based, at least in part, on an obesity analyte signature in the sample. For example, a urine sample can be obtained from an obese mammal and can be assessed to determine if the obese mammal is likely to be responsive to pharmacological intervention based, at least in part, on an obesity phenotype, which is based, at least in part, on an obesity analyte signature in the sample.

### Methods of Detection

Any appropriate method can be used to detect the presence, absence, or level of an analyte provided herein (e.g., an obesity analyte) within a sample. For example, mass spectrometry (e.g., triple-stage quadrupole mass spectrometry coupled with ultra-performance liquid chromatography (UPLC)), radioimmuno assays, enzyme-linked immunosorbent assays, hybridization assays, amplification assays (e.g., PCR and/or RT-PCR), sequencing techniques (e.g., PCR-based sequencing techniques), and/or restriction fragment length polymorphism (RFLP) can be used to determine the presence, absence, or level of one or more analytes in a sample.

### EXAMPLES

The present disclosure is further illustrated by reference to the following Examples. However, it should be noted that these Examples, like the embodiments described above, are illustrative and are not to be construed as restricting the scope of the invention in any way.

### Example 1—Biomarkers for Prediction of Weight Loss in Obesity and Diabetes

### Objective

The objective of this Example was to elucidate food intake regulation and energy expenditure aspects of energy balance in human obesity pathophysiology and describe a classification method to further understand the unique characteristics and actionability of these phenotypes in human obesity.

### Methods and Results

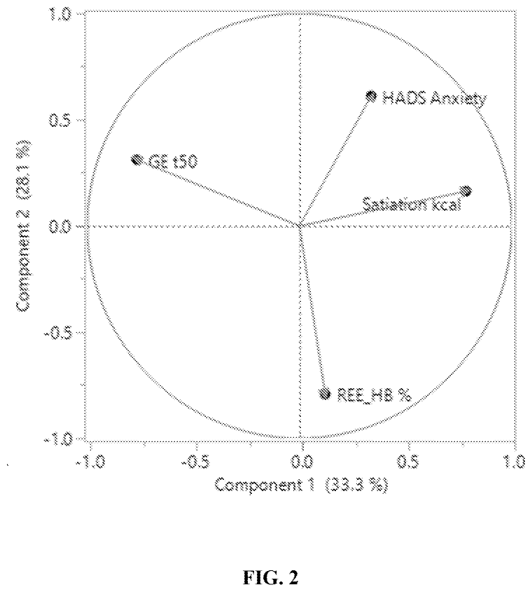

To address this objective, specific characteristics of a cohort of patients with obesity (defined as BMI>30 kg/m²) were prospectively studied and classified by their predominant obesity-related phenotype. The overall cohort included 120 Caucasian participants with the following demographics (median (IQR)): age 36 (28-46) years, BMI 35 (32-38) kg/m2, and 75% females. All participants completed the following validated tests: a) satiation, studied by ad libitum buffet meal (kcal consumed to reach maximal fullness; calories to fullness (CTF)) and visual analog scale for fullness (100 mm scale) at baseline and postprandial every 15 minutes for 2 hours, b) satiety, studied by visual analog scale for appetite (100 mm scale) at baseline and postprandial every 15 minutes for 2 hours after a standard 300 kcal meal and gastric emptying of solids (summarized by the half-emptying time, T1/2, minute), c) hedonic, studied by hospital anxiety and depression score (HADS) and Three Eating Factor Questionnaires (see US20210072259, which is incorporated by reference in its entirety for all purposes), and d) energy expenditure, studied by resting energy expenditure (REE) (indirect calorimetry), non-exercise physical activity and exercise. Based on the results of these variables, energy expenditure variables were added to those previously evaluated, and unsupervised principal component analysis (PCA) was conducted to gauge the contribution of these factors to the variance of obesity. The PCA findings confirm resting energy expenditure as a new latent dimension (FIG. 2).

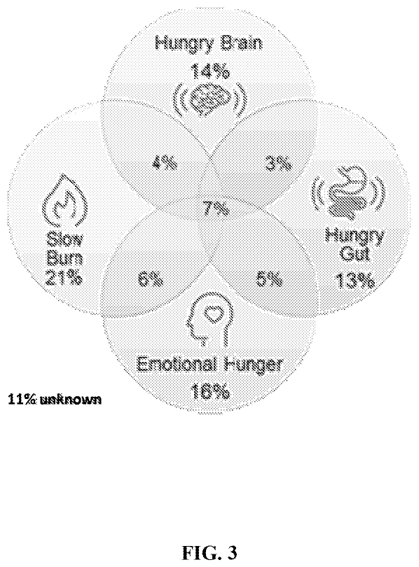

Then, with the intention to translate the unsupervised obesity-related phenotype PCA to quantifiable and reproduce specific a priori determined cutoff, the 75th percentile of the median each measurement in females and males for each phenotype was used as a cutoff to identify prevalence of the five distinct phenotypes among the patients with obesity: hungry brain—abnormal satiation (14.2%), hungry gut—abnormal satiety (12.5%), emotional hunger—hedonic eating (15.9%), slow burn—abnormal metabolism (20.8%); mixed (25.8%) and other, that is 10.8% in whom none of the previously identified phenotypes was observed (FIG. 3). Among these phenotypes there was no statistical difference in body weight, waist circumference, or hip circumference.

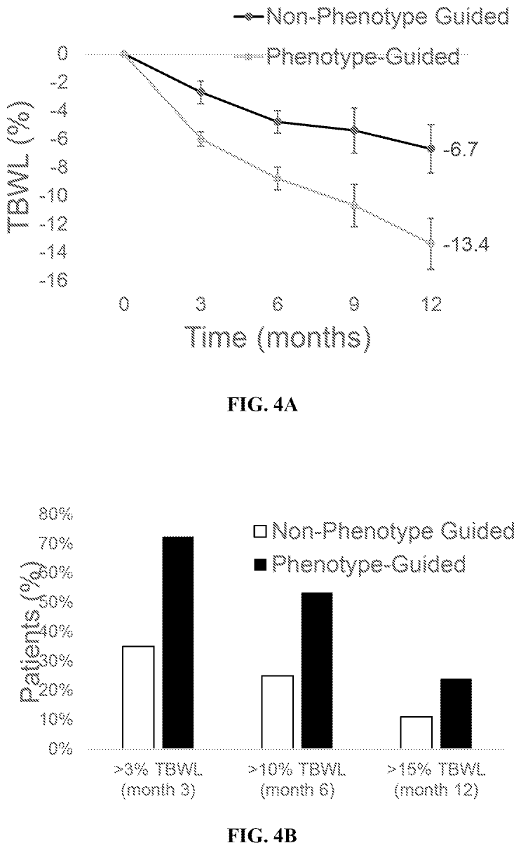

In a case-control prospective trial with data collected retrospectively, a phenotype-guided pharmacotherapy was applied (intervention group (n=55)) and compared to standard of care, physician selected pharmacotherapy (control group (n=175)) in patients with obesity. Results showed that phenotype-guided pharmacotherapy doubles the weight loss at 12 months of treatment (Intervention group 12.9±1.9% total body weight loss (TBWL) compared to 6.7±1.2% TBWL in standard of care group, p<0.0025; FIG. 4A). Moreover, the intervention group had 74% responders (defined >3% TBWL in 1st month) compared to 33% in controls (FIG. 4B).

### Example 2: Multi-Omics, Fasting, Blood-Based Biomarker Predicts Obesity Phenotypes Using a Machine Learning Model-Initial Study

### Objective

A multi-omics approach (GWAS, targeted metabolomics and hormones) was used to identify blood-based multi-omics biomarkers that can be used to determine an obesity phenotype which can be used to predict weight loss in response to obesity interventions. Advanced statistical techniques were used to identify multi-omics based biomarkers which can predict the 4 obesity phenotypes with >80% sensitivity and specificity.

### Methods

Patients from two different cohorts have been phenotyped. In the first cohort, a total of 274 patients were phenotyped. All were overweight or obesity. GWAS, GI hormones and targeted metabolomics were completed in 181 patients. The phenotype distribution from the first cohort is shown in Table 1.

TABLE 1
Cohort 1 (181 Patients).

| | | >2 | # | | >90% | #pts | % | >75% | #pts | % |
|---|---|---|---|---|---|---|---|---|---|---|
| Trait | Mean + SD | SD | pts | Median | ile | w/trait | pts | ile | w/trait | pts |
| HADS-A | 3.4 + 2.5 | 9 | 17 | 3 | 7 | 29 | 16% | 6 | 46 | 25% |
| SGE T1/2 | 99.5 + 25.8 | −47 | 5 | 98 | 70.8 | 17 | 9% | 81 | 43 | 24% |
| Buffett | 917 + 295 | 1604 | 16 | 916 | 1357 | 23 | 13% | 1184 | 44 | 24% |

In the second cohort, a total of 165 patients were phenotyped. All have obesity. GWAS, GI hormones and targeted metabolomics were completed in 88 patients. The phenotype distribution from the second cohort is shown in Table 2.

TABLE 2
Cohort 2 (88 Patients).

| Obesity | | | Results | | |
|---|---|---|---|---|---|
| Categories | Phenotype | Test | All Cohort | Females | Males |
| Food Intake - | Satiation | Ad Libitum Buffett | 872.8 ± 28.9 | 777.2 ± 22.4 | 1136 ± 71.4 |
| Homeostatic | | meal, Kcal | | | |
| | | VAS - Satisfaction 30 | 83.2 ± 2.1 | 83 ± 2.5 | 83.6 ± 4 |
| | | min postprandial, mm | | | |
| | Satiety | VAS - Fullness 120 | 61.8 ± 2.3 | 61.2 ± 2.8 | 63.4 ± 3.9 |
| | | min postprandial, mm | | | |
| | | Gastric Emptying T ½, | 118.7 ± 2.5 | 125 ± 2.8 | 101.3 ± 4.4 |
| | | min | | | |
| Food Intake - | Emotional Eating | TEFQ - Emotional | 8.5 ± 0.4 | 8.8 ± 0.4 | 7.7 ± 0.7 |
| Hedonic | | restraint (4-16 Scale) | | | |
| Eating | | HADS-A (0-21 scale) | 4.4 ± 0.26 | 4.4 ± 0.3 | 4.4 ± 0.6 |
| Energy | Basal Metabolic | Predicted REE (HB) % | 101.7 ± 1 | 102.9 ± 1 | 98.2 ± 2.9 |
| Expenditure | rate | | | | |
| | Non-Exercise | Self-Reported Steps, # | 5881 ± 327.2 | 5607 ± 354 | 6741 ± 764.3 |
| | Physical Activity | | | | |
| | Exercise | Self-Reported Exercise | 6.1 ± 0.1 | 6 ± 0.1 | 6.3 ± 0.2 |
| | | (PASC), 0-8 scale | | | |

### Statistical Summary:

1. Initial analysis was performed using only metabolomics. A multi-nominal logistic regression was used to identify predictors of response in the first cohort. This approach was not validated in the cohort.
2. Biomarker discovery for phenotypes (hungry brain—buffet test, hungry gut—Get1/2, emotional hunger—HADS-a). Using multinominal logistic regression analysis and bootstrapping.

A. Summary: negative results

B. Comments:
i. used 17 SNPs.
ii. No GI hormones in validation cohort
iii. Difference among the two cohorts

3. GWAS analysis against healthy controls: completed the GWAS of obese cases versus biobank controls. He completed 2 analyses.
A. Analysis 1: all cases (phenotypes 1-5) versus controls using logistic regression
B. Analysis 2: phenotypes 1-3 versus controls using multinomial logistic regression

4. GWAS and metabolomics of Q! vs Q4

5. GWAS and metabolomics “quantitative” analysis against the phenotypes

6. PCA and clustering analysis.

### Statistical Methods

The experimental design consisted of two cohorts. The first cohort (cohort #1) included 167 patients. The second cohort (cohort #2) included 106 patients.

Three endpoints were evaluated: (i) buffet meal, (ii) SGET, (iii) HADS and (iv) REE. Distributions of these endpoints were stratified by sex and cohort.

The following variables were considered in order to develop multivariable models to predict each of the three endpoints. The metabolite data were centered and scaled within each of the two cohorts separately.

Three different prediction methodologies were evaluated: (i) LASSO regression, (ii) classification and regression trees (CART), and (iii) gradient boosting machine (GBM), which is a machine learning technique for regression and classification problems. In developing the prediction models, the two cohorts were combined, and 10-fold cross validation was utilized to evaluate prediction performance of each of the models. The rational for combining the two cohorts versus treating them as a discovery (cohort #1) and replication (cohort #2) cohort, was because the endpoints had different distributions across the two cohorts. The assumption is that this reflects the inherent variability in the data, and a more representative sample would be obtained by combining the two cohorts.

To evaluate predictive accuracy, calibration and discrimination were evaluated. Calibration refers to unbiased prediction estimates. Plots of predicted versus observed values were used to evaluate calibration. A linear regression model was fit to these plots, y=a+bx, where a denotes if the prediction is systematically an under/over-estimate, and for an unbiased prediction model, b=1. Discrimination measures a predictor's ability to separate patients with different responses. Root mean squared error (RMSE) and c-index were used to measure discrimination. The squared error is defined as the squared difference between predicted and observed values; the values are squared in order to eliminate negative values. The average is taken across all observations. The square root is subsequently taken in order to put the values back on the original scale. Thus, the RMSE denotes the average difference between the observed and predicted values. The c index estimates the probability of concordance between predicted and observed responses. A value of 0.5 indicates no predictive discrimination and a value of 1.0 indicates perfect separation of patients with different outcomes.

$$R M S E = \sqrt{\frac{{\sum }_{i = 1}^{N} {( {Predicted}_{i} - {Actual}_{i} )}^{2}}{N}}$$

### Results

A two-stage design was used; the training (n=180) and validation (n=120) cohorts.

Variables included:
A. Behavioral Questionnaires (i.e., Hospital Anxiety and depression scale (HADS); Three Eating Facto Questionnaire (TEFQ)).
B. Candidate genes (n=17 SNPs; rs1800544; rs2234888; rs7903146; rs9939609; rs17782313; rs5443; rs1129649; rs1047776; rs659366; ucp2; rs2075577; rs15763; rs1626521; rs4795541; rs3813929; rs518147; rs1414334; rs11554825; rs1800544).
C. Targeted Metabolomic (n=50; hydroxyproline; methylhistidine1; methylhistidine3; asparagine; phosphoethanolamine; arginine; taurine; serine; glutamine; ethanolamine; glycine; asparticacid; sarcosine; citrulline; glutamicacid; beta.alanine; threonine; alanine; gamma.amino.n.butyricacid; alpha.aminoadipicacid; beta.aminoisobutyricacid; proline; hydroxylysine1; hydroxylysine2; alpha.amino.N.butyric.acid; ornithine; cystathionine1; lysine; cystine; tyrosine; methionine; valine; Isoleucine; allo.isoleucine; leucine; phenylalanine; tryptophan; acetylcholine; histidine; serotonin; acetic; propionic; isobutyric; butyric; isovaleric; isocaproic; hexanoic).
D. GI satiety hormones (n=4)

Three machine learning—prediction methodologies were evaluated:
1. LASSO regression,
2. Classification and regression trees (CART), and
3. Gradient boosting machine (GBM), which is for regression and classification problems.

In developing the prediction models, the two cohorts were combined, and 10-fold cross validation was utilized to evaluate prediction performance of each of the models.

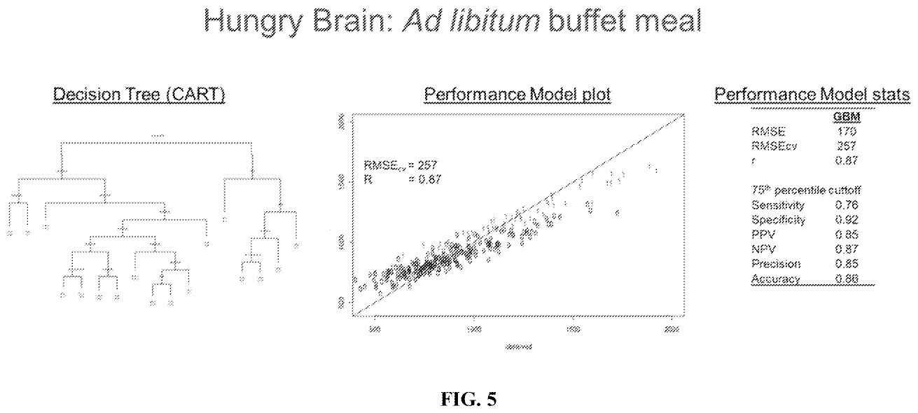

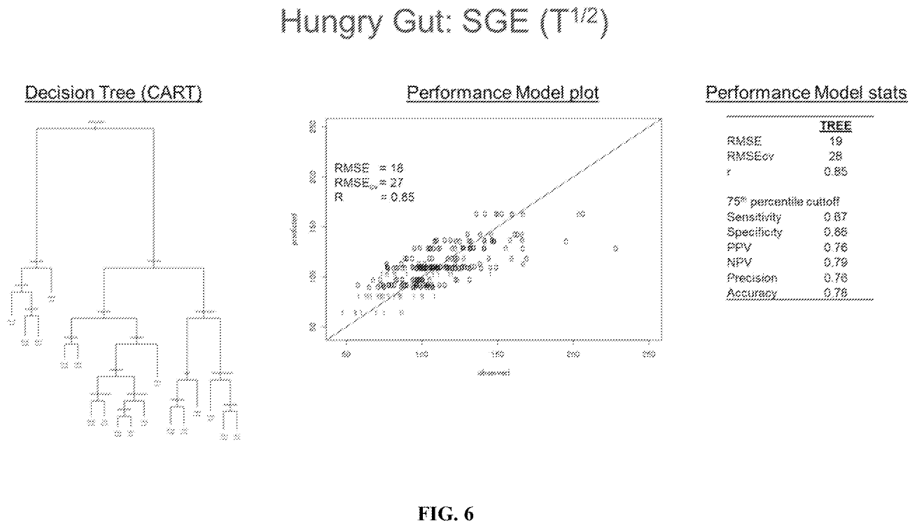

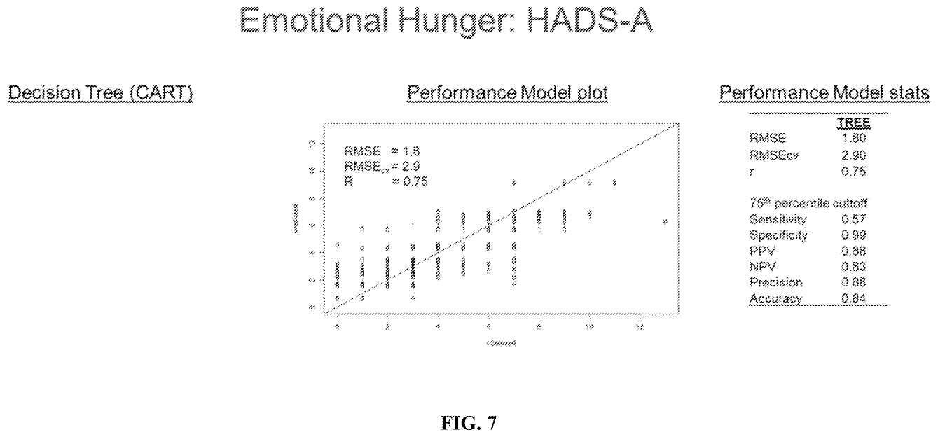

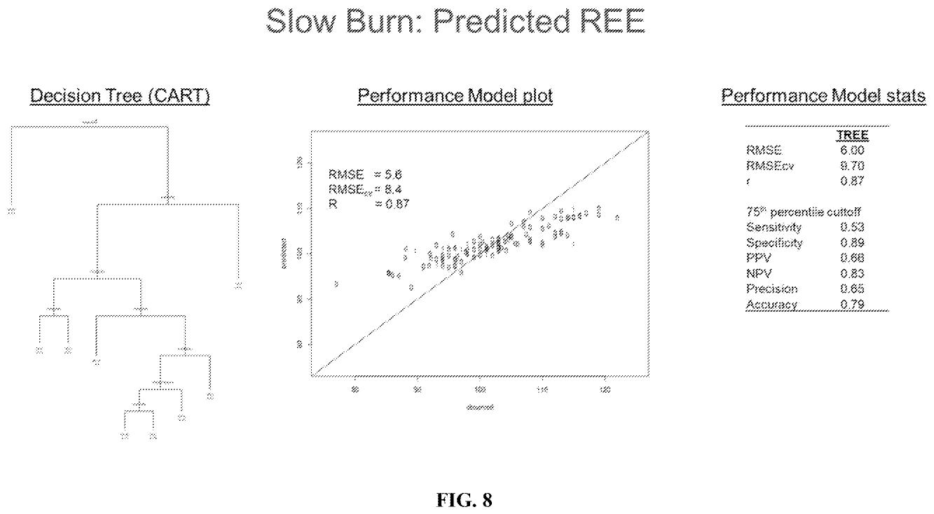

Predictors and accuracy were retained in 100 bootstrapped samples testing. FIGS. 5-8 represent the results and performance of the GBM and CART models for hungry brain, hungry gut, emotional hunger, and slow burn, respectively.

### Example 3: Multi-Omics, Fasting, Blood-Based Biomarker Predicts Obesity Phenotypes Using a Machine Learning Model-Follow-Up Study

### Objective

Pathophysiological and behavioral obesity phenotypes explain the heterogeneity of human obesity, predict weight gain, inform anti-obesity medication (AOM) selection and enhance AOM weight loss response, and also predict tolerability and weight loss for bariatric endoscopy. The predominant obesity phenotypes are: (i) abnormal satiation, (ii) abnormal postprandial satiety, (iii) emotional eating, and (iv) abnormal resting energy expenditure. However, the tests that measure obesity phenotypes are currently limited to a few research/academic centers. The goal of this Example was to identify blood-based multi-omic (demographics, GWAS, targeted metabolomics and hormones) novel biomarker(s) that can predict the obesity phenotypes in human obesity. To achieve this goal, advance statistical techniques were applied to identify a multi-omics based biomarker that predict the four (4) obesity phenotypes with >80 sensitivity and specificity.

### Methods

273 participants had the following phenotype tests performed: satiation by ad libitum buffet meal (kcal); postprandial satiety by VAS fullness (mm) and gastric emptying with scintigraphy (min); emotional eating by questionnaires (TEFQ, HADS); and resting energy expenditure by indirect calorimetry (kcal/24 hours). A fasting blood sample was collected for satiety GI hormones (ELISA), metabolomics (Mass Spectrometry), and DNA (SNP Array) (see Example 2). Two different machine learning prediction methodologies were evaluated for predicting the phenotypes of interest: (i) classification and regression trees (CART) and (ii) gradient boosting machine (GBM). 10-fold cross validation was utilized to evaluate prediction performance, and cross-validated root mean square error (RMSE_{CV}), correlation between observed and predicted outcomes (r), and precision/accuracy of predicting the 75th percentile for each endpoint was calculated.

### Results

A total of 273 participants-samples were included in the analysis (age 37±11 years, 76% females, BMI 37±5 kg/m²). The model included the following a priori chosen variables: 7 demographics, 10 germline variants, 4 hormones and 39 targeted metabolomics. Using the GBM model, the multi-omic biomarker performance for predicting satiation was: RMSE_{CV}=257, r=0.87, precision of 85% and accuracy of 86%. Using the CART model, the multi-omic biomarker performance for postprandial satiety was: RMSE_{CV}=28, r=0.74, precision of 76% and accuracy of 78%. The CART model for emotional eating had RMSE_{CV}=2.9, r=0.75, precision of 88% and accuracy of 84%. The CART model for resting energy expenditure had RMSE_{CV}=9.7, r=0.72, precision of 65% and accuracy of 79%. The GBM and CART models outperformed multinomial logistic regressions and individual variables (e.g., genetics variants alone).

These results demonstrate the identification of a fasting, blood-based biomarker for obesity phenotypes. This novel multi-omic biomarker, driven by phenotypes, and developed by machine learning algorithms, can be used for reducing the variability in treatment response in the management of obesity.

### Example 4: Genetic Variants Associated with Accelerated Gastric Emptying in Patients with Obesity

### Objective

Gastric emptying controls the timing and rate of emptying food and is a critical mediator of satiety and food intake regulation. Accelerated gastric emptying is a trait seen in human obesity. Furthermore, it is associated with increased weight gain in young adults. Genetic factors play a crucial role in an individual's predisposition to obesity, and current evidence has associated a multitude of single-nucleotide polymorphisms (SNPs) with body mass index (BMI) and adiposity. However, the influence of genetics on other obesity-related traits remains uncertain. This Example describes the identification of specific genetic variants associated with gastric emptying in patients with obesity.

### Methods

Gastric emptying was measured and a genome-wide association study (GWAS) of venous blood samples in a total of 259 patients with obesity (age 37±11 years, 76% females, BMI 37±5 kg/m²) was performed. Gastric emptying was measured by scintigraphy after a standard 320 kcal, 30% fat meal consisting of two ^{99m}Tc-radiolabeled eggs, toast, and 80 mL of milk. Genotyping was performed using the Infinium Omni2.5Exome-8 BeadChip Array. 16,145 single nucleotide polymorphisms (SNPs) known to be associated with obesity were evaluated a priori for the current study. Associations with gastric emptying of solids were explored using a linear regression analysis adjusted for age and sex. SNPs with a β>10 minutes and a p-value <1×10⁻³ were included in the pathway analysis.

### Results

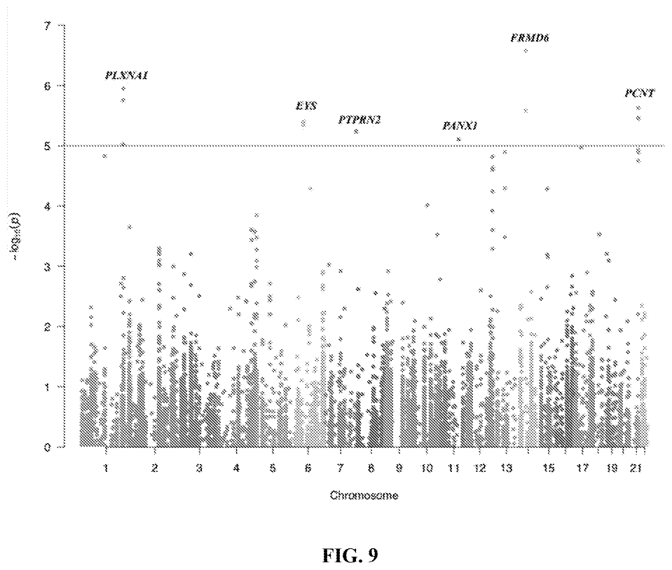

Although none of the SNPs in the cohort achieved genome-wide significance (p<1×10⁻⁸), a total of 7 SNPs showed a statistically significant association with gastric emptying (p<1×10⁻⁶) (Table 3, FIG. 9). The rs1885034 SNP in the FRMD6 gene showed the strongest association with accelerated gastric emptying (β=11.89; SE=2.247; P=2.62×10⁻⁷). A total of 43 SNPs associated with an accelerated gastric emptying are involved in the following pathways: insulin uptake TCERG1L, PANX1 and PTPRN2; lipid metabolism ADH1B, HHAT, LIPC, LRP1B, and RARB; cell cycle CNOT2, FHIT, and PCNT; G-protein coupled receptors signaling AP2B1 and RGS9; cell differentiation and proliferation C8ORF37, ERBB4, PRKN, NTRK2, EYS and PARK2; Hippo signaling FRMD6, Axon guidance PLXNA1; and protein modification GLTID1.

TABLE 3
Pathway GENE/SNP association with gastric emptying.

| PATHWAY | GENE | SNP | A₁ | A₂ | β min | SE | p |
|---|---|---|---|---|---|---|---|
| Axon guidance | PLXNA1 | rs1664232 | T | C | 10.48 | 2.141 | 1.76 × 10⁻⁶ |
| | | rs11118997 | G | A | 9.543 | 2.11 | 9.39 × 10⁻⁶ |
| Celld ifferentiation | EYS | rs9342434 | A | G | 14.79 | 3.136 | 3.97 × 10⁻⁶ |
| and proliferation | | | | | | | |
| Insulin uptake | PTPRN2 | rs2335852 | G | A | 22.7 | 4.902 | 5.79 × 10⁻⁶ |
| | PANX1 | rs11020655 | A | G | 15.62 | 3.424 | 7.84 × 10⁻⁶ |
| Hippo signaling | FRMD6 | rs1885034 | C | T | 11.89 | 2.247 | 2.62 × 10⁻⁷ |
| Cell cycle | PCNT | rs7277175 | G | A | 18.14 | 3.755 | 2.36 × 10⁻⁶ |

SE: Standard Error

These results demonstrate that several physiologically-relevant genes can be associated with the gastric emptying rate of solids in patients with obesity. Genetic variations may influence gastric emptying rate, significantly affecting postprandial satiety in patients with obesity.

### Example 5: Impact of Gastric Emptying and Genetic Variants Related to GLP-1 on Weight Loss with Liraglutide in Treatment of Obesity-Pilot Study

### Objective

Three (3) mg subcutaneous (SQ)/day of liraglutide or placebo were administered for 16 weeks in order to compare the effects of liraglutide on gastric emptying (GE), gastric accommodation (GA), satiation, and buffet meal intake in patients with obesity. To assess baseline characteristics including GE T_{1/2} as covariates on weight loss with liraglutide and the influence of variants rs6923761 GLP-1 receptor and rs7903146 TCF712 (regulator of proglucagon gene in enteroendocrine L cells).

### Methods

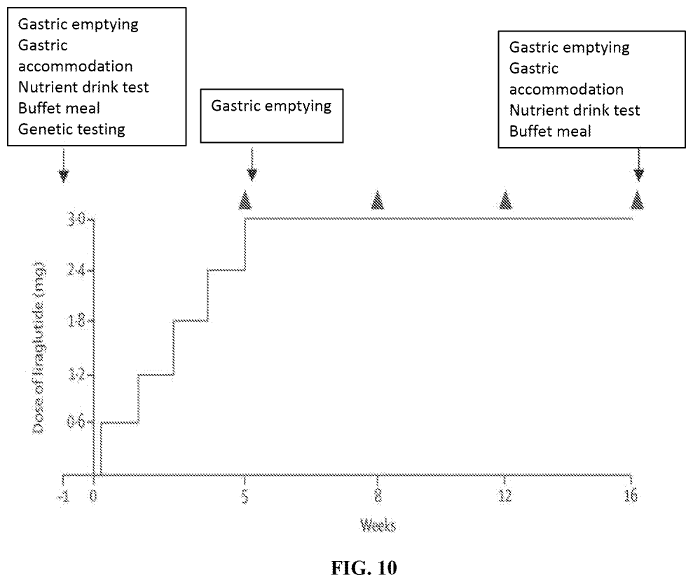

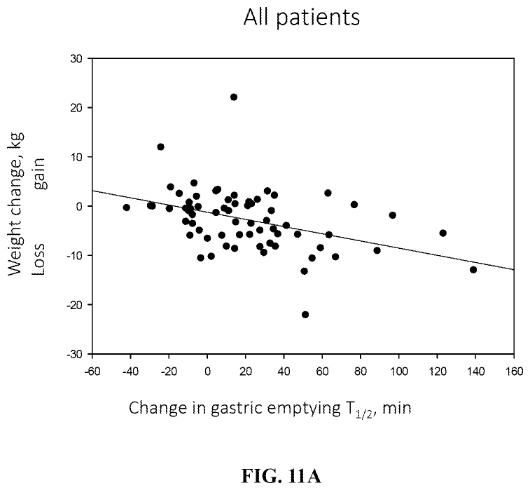

In a randomized, placebo-controlled trial of liraglutide, 82 participants with obesity (BMI≥30 kg/m²) received nutritional and behavioral counseling and followed standard dose escalation by 0.6 mg liraglutide/day each week for 5 weeks. Liraglutide or placebo (saline) was self-administered SQ as identical volumes once per day at the maximum tolerated dose for the next 11 weeks. All participants included underwent, at baseline and after 16 weeks of liraglutide/placebo, the following measurements: GE of solids (320 kcal, 30% fat) by scintigraphy over 4 hours; gastric volumes (fasting and post-300 mL Ensure®) by ^{99m}Tc-SPECT; satiation (kcal to fullness (CTF)) and maximum tolerated calories (MTC) with nutrient drink test (Ensure® 30 kcal/minute); and kcal intake at an ad libitum buffet meal. Gene variants were studied by PCR using Taqman® SNP genotyping assays. See FIG. 10 for an illustration of the study design. Statistical analysis (non-parametric except for weight and waist circumference) compared liraglutide to placebo using ANCOVA with baseline measurements as covariates.

### Results

There were 6 drop-outs; complete data were available for 76 participants; 73 patients were receiving maximum dose at the end of 16 weeks. Patients' demographic data, baseline measurements, and gastric motor functions, satiation and satiety parameters with treatment are shown in Table 4. As shown in Table 4, compared with those receiving placebo injections, those receiving liraglutide had a greater loss of weight after 16 weeks. Subjects in the liraglutide arm lost 5.6±5.3 kg over the 16 weeks, compared to −0.1±5.6 kg in the placebo arm. This achieved statistical significance when analyzed with sex and baseline weight as covariates. Additionally, regarding the effects on gastric motor function, those in the liraglutide arm experienced a significantly delay in gastric emptying compared to the placebo arm, with time to half emptying increased by nearly 37 minutes (see Table 4). Conversely, liraglutide did not induce change in fasting gastric volumes or change in gastric accommodation volumes from baseline to 16 weeks compared with placebo (see Table 4). Kilocalories until fullness with a standardized liquid nutrient drink test decreased by a mean of 133 kCal after 16 weeks of treatment in the liraglutide arm, with no significant change in the placebo arm. However, liraglutide induced no change at 16 weeks in maximum tolerated kCal or kCal consumed at a buffet meal (see Table 4).

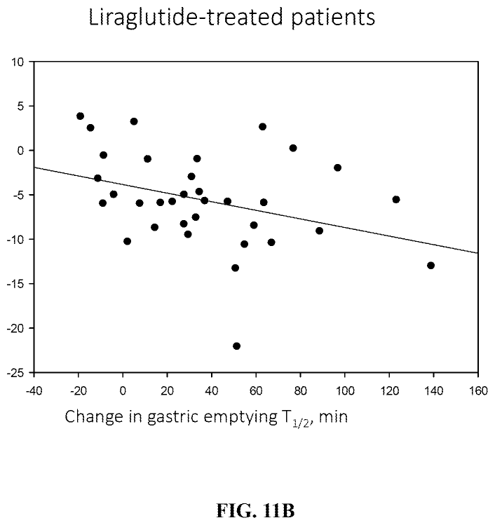

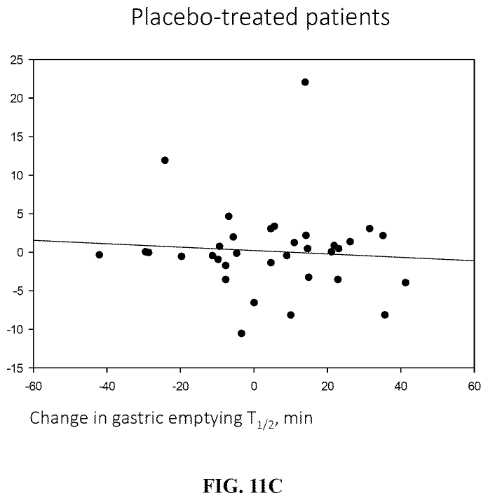

When examining all study subjects, there was an association between greater weight loss with greater delay in gastric emptying (see FIG. 11A). A trend towards the same association was observed in the liraglutide arm but not in the placebo arm (see FIGS. 11B-11C). As such, there appears to be a relationship of change in GE T1/2 and weight loss over 16 weeks of treatment.

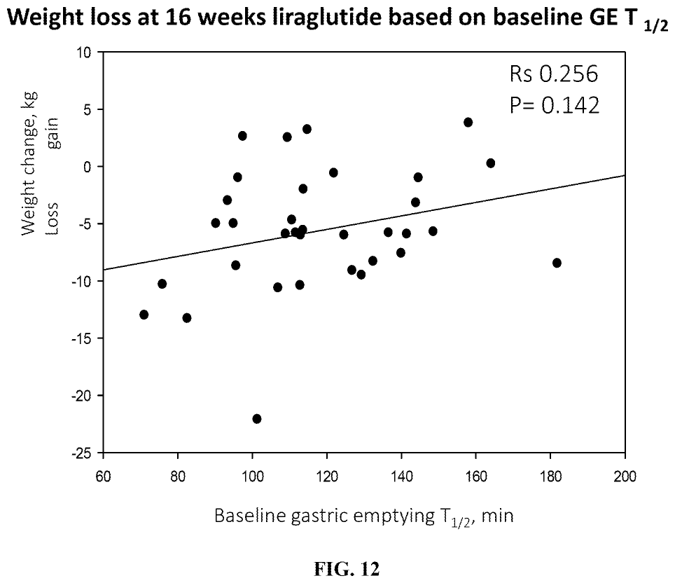

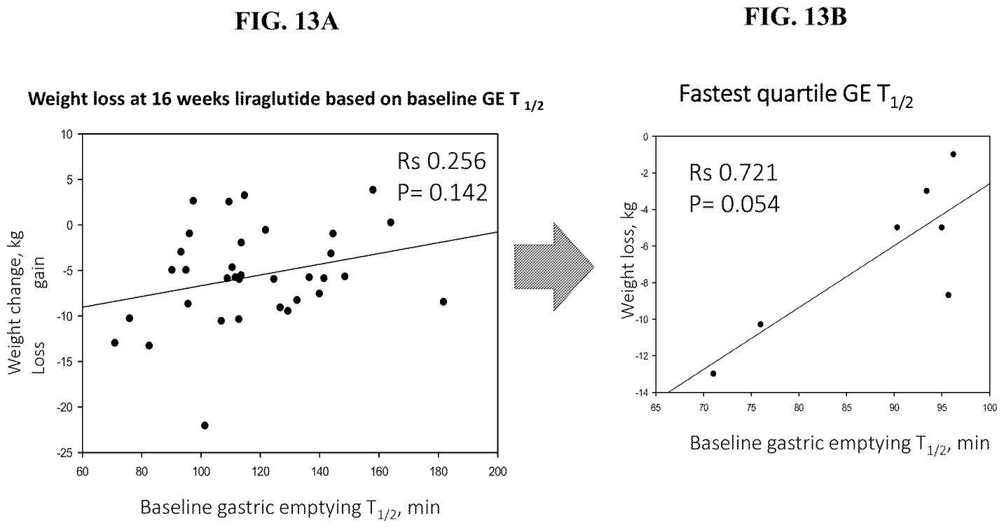

Given the trend towards greater weight loss with greater delaying in GE for those receiving liraglutide, the baseline gastric emptying influence on weight loss with liraglutide treatment was examined. When looking at the entire liraglutide cohort, there was not a correlation with baseline gastric emptying and weight loss as shown in FIG. 12. However, when examining the fastest quartile of GE in those receiving liraglutide, there was a trend towards greater weight loss in those with faster baseline GE rates as shown in FIGS. 13A-13B.

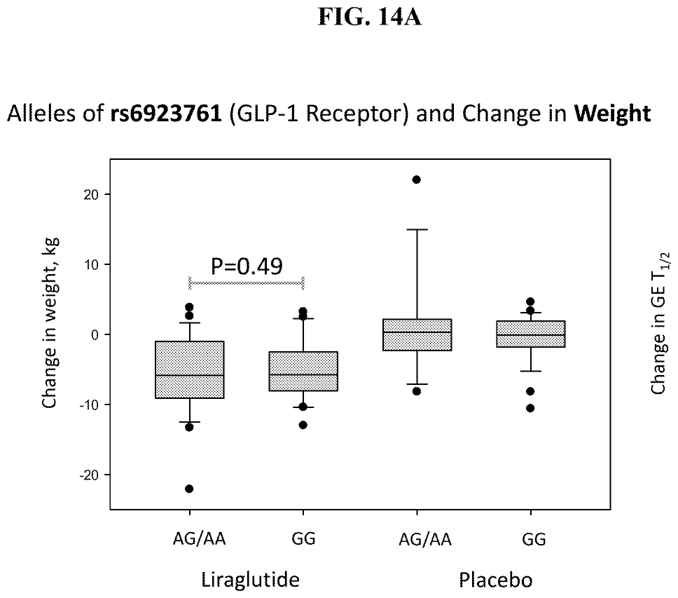

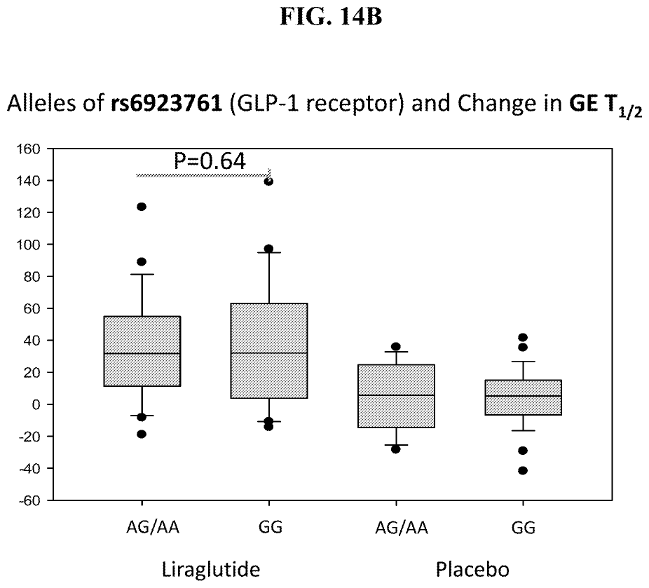

As shown in FIGS. 14A-14B, allelic variations in GLP1R did not influence weight loss or change in GE T1/2 induced by liraglutide. For patients receiving liraglutide, there was no observable difference in change in weight FIG. 14A) or change in gastric emptying (FIG. 14B) over 16 weeks when separating subjects by allele of the GLP-1 receptor.

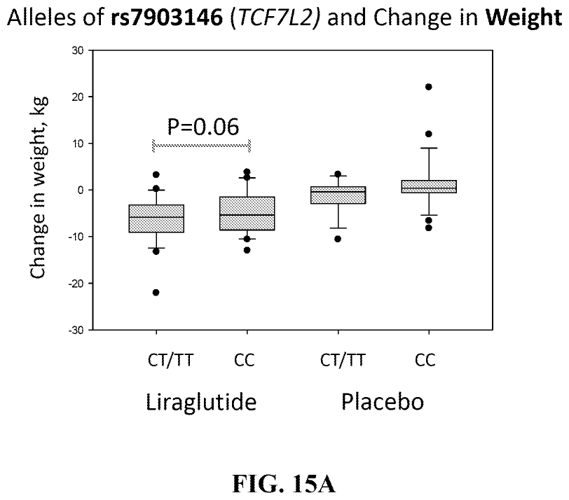

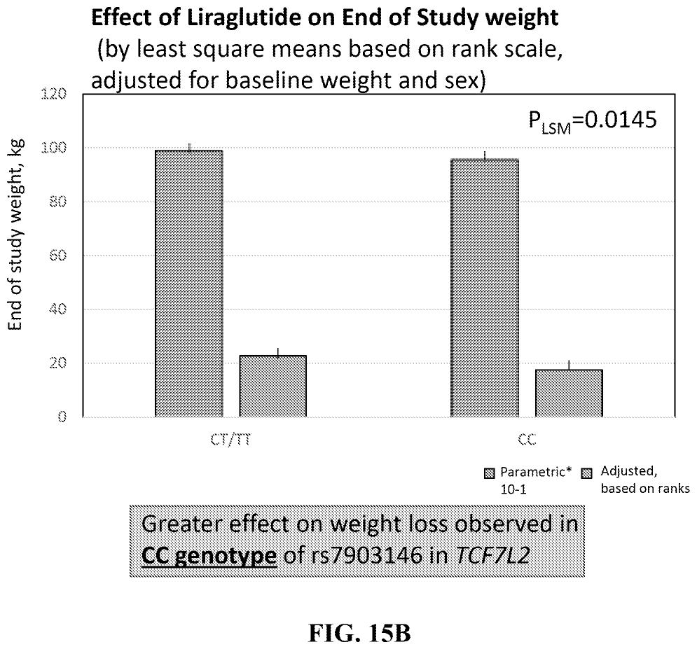

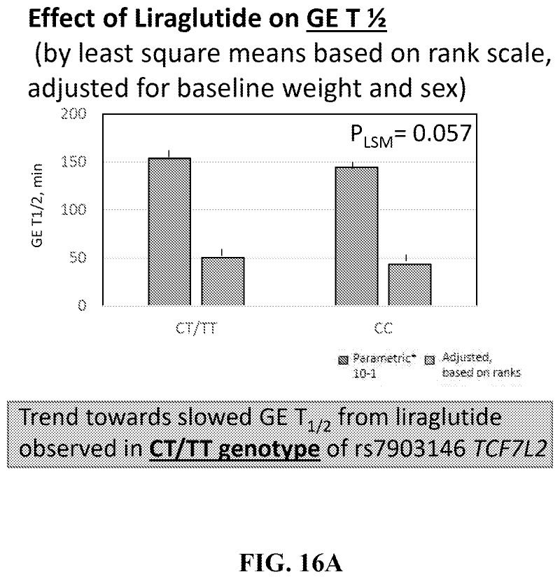

As shown in FIGS. 15A-15B, allelic variations in TCF7L2 influence weight loss effect by liraglutide. When looking at the unadjusted results for change in weight based on TCF7L2 allele, there appeared to be a trend towards a gene-by-treatment effect for the CT/TT vs CC allele for liraglutide. Using a least square means adjusted model for baseline weight and sex, end of study weight was lower for those treated with liraglutide if they harbored the CC genotype.

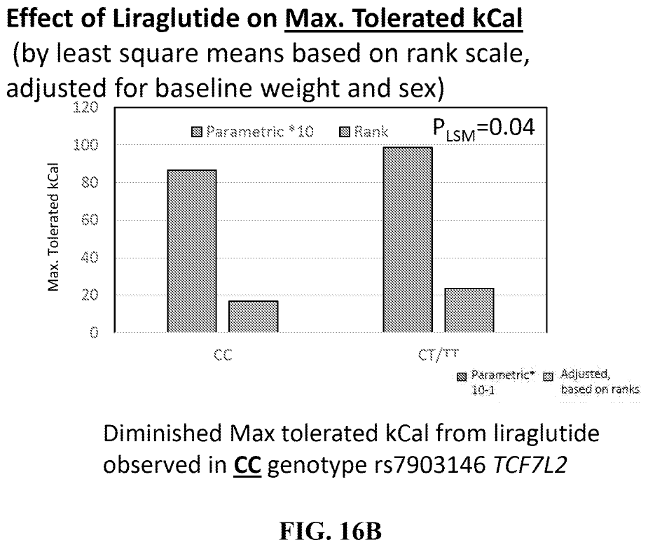

With the observable end of study weight difference in the CC genotype of TCF7L2 using a least squared means adjusted model, an exploration on if there was a physiologic underpinning to this gene-by-treatment effect was conducted. Given the association of delay in gastric emptying and weight loss previously described, it was hypothesized that this gastric motor function may underly the greater end of study weight observed in the CC genotype of TCF7L2; however, a trend towards more delayed gastric emptying was observed in the alternate genotype (see FIG. 16A). With regard to the other gastric motor functions and measures of appetite, it was observed that the CC genotype of TCF7L2 was associated with more diminished maximum tolerated kCal of a nutrient drink test by end of study (see FIG. 16B). This finding suggests that a plausible mechanism for the lower end of study weight achieved by those getting liraglutide with the CC genotype may be partially driven by a greater responsiveness to calories ingested during a single meal.

### Conclusions

The following were significant predictors of weight loss on liraglutide: baseline GE T_{1/2} (P<0.001) and CTF (P=0.044) and TCF7L2 rs7903146 (p=0.0145). There were significant gene-by-treatment interactions for specific endpoints: GLP1R rs6923761 for waist circumference (p<0.01), and TCF7L2 rs7903146 for waist (p=0.02), CTF (p=0.035) and GE T_{1/2} (p=0.057). Variants in GLP1R rs6923761 and TCF7L2 rs7903146 were found to be associated with weight loss or on the effects of liraglutide on GE and CTF. Allelic variations in rs7903146 (TCF7L2) predict clinical response: greater effect on weight loss from liraglutide in CC genotype and greater effect on gastric emptying in CT/TT genotype.

TABLE 4
Effect of 16 weeks' treatment with liraglutide.

| | | | | | | | | | Calories | | |
|---|---|---|---|---|---|---|---|---|---|---|---|
| | | | | | Waist | GE | Fasting | Accom. | to | Maximum | Buffet |
| Group | | | Age | Weight | Circ. | T_{1/2}, | GV, | Vol, | fullness, | tolerated | meal, |
| Mean (SD) | N/ | F | years | kg | cm | min | mL | mL | kcal | kcal | kcal |
| Liraglu: Base | 39 | 34 | 39.9 | 102.1 | 113.8 | 121.3 | 192.1 | 348.1 | 692 (266) | 1223 (360) | 901 |
| | | | (9.1) | (13.0) | (10.3) | (26.4) | (39.6) | (81.8) | | | (297) |
| Liraglu: Rx | | | | 97.2 | 108.4 | 154.7 | 223.3 | 371.6 | 547 (180) | 921 (272) | 736 |
| | | | | (14.0) | (9.4) | (42.4) | (59.3) | (69.5) | | | (331) |
| LIRAGLU Δ | | | | −5.6 | −6.2 | 36.8 | 35.9 | 32.0 | −133 (221) | −284 (348) | −184 |
| | | | | (5.3) | (7.2) | (38.3) | (56.2) | (96.8) | | | (205) |
| Placebo: | 37 | 32 | 37.3 | 102.7 | 109.8 | 108.4 | 206.5 | 360.5 | 784 (313) | 1293 (359) | 949 |
| base | | | (9.9) | (15.6) | (11.8) | (22.2) | (54.9) | (99,7) | | | (330) |
| Placebo: Rx | | | | 102.5 | 110.4 | 113.7 | 207.2 | 379 | 692 (263) | 1423 (1800) | 797 |
| | | | | (15.5) | (12.8) | (26.3) | (60) | (105.7) | | | (241) |
| PLACEBO Δ | | | | 0.1 (5.6) | 1.5 (7.3) | 4.5 | 1.9 | 15.5 | −84 (241) | 139 (1808) | −130 |
| | | | | | | (20.1) | (75.7) | (121.6) | | | (168) |
| P ANCOVA | | | | 0.0077 | 0.0080 | 0.0001 | 0.099 | 0.411 | 0.044 | 0.093 | 0.301 |

### Example 6: Impact of Gastric Emptying and Genetic Variants Related to GLP-1 on Weight Loss with Liraglutide in Treatment of Obesity-Follow-Up Study

### BACKGROUND

At a dose of 3 mg/day administered subcutaneously (SQ), liraglutide, a long-acting GLP-1 receptor agonist with 97% homology to human GLP-1, is FDA-approved for weight management in adults with BMI≥30 kg/m², or ≥27 kg/m² with obesity related co-morbidities, and for adolescents aged 12 to 17 years with a body weight of at least 60 kg and BMI≥30 kg/m². A recent network meta-analysis showed that SQ liraglutide >1.8 mg dose is one of the three most effective GLP-1 receptor agonists for weight loss.⁹

The mechanistic underpinnings of liraglutide in treatment of obesity are likely multifold. In a prior pilot trial of the first 40 participants in this 136-person study, 3 mg liraglutide significantly delayed gastric emptying of solids at both 5 and 16 weeks, and this delay correlated with weight loss.¹⁰ Liraglutide use is associated with nausea which may result from retardation of gastric emptying. In addition, endogenous GLP-1 slows gastric emptying as demonstrated by administration of the specific GLP-1 antagonist, exendin(9-39) amide.¹¹ GLP-1 itself increases fasting and postprandial gastric volumes.¹² Thus, an effect of liraglutide on gastric accommodation (indirectly affecting appetite) could also contribute to the weight loss. Given that the pilot study involved 40 participants and was underpowered to detect effects on functions such as gastric accommodation and the potential pharmacogenomic interactions with genetic variation in GLP-1R (receptor gene) and the TCF7L2 that controls endogenous GLP-1 synthesis, it is important to complete a randomized, controlled trial as described in this Example.

GLP-1 activity is mediated by a complex pathway of genes and their products including the product of the transcription factor 7-like 2 gene (TCF7L2) which drives transcription of pre-proglucagon in enteroendocrine L cells. GLP-1 signals through its cognate receptor (encoded by GLP1R). rs6923761 in GLP1R is associated with altered response to GLP-1.¹³ The A allele (AA/AG) in comparison to GG genotype showed greater effects of liraglutide 1.8 mg/day on BMI, body weight, and fat mass.¹⁴

TCF7L2rs7903146 is associated with defects in insulin secretion and type 2 diabetes mellitus,^{13,15} and with more rapid gastric emptying of liquids with the CT/TT genotypes compared to CC group.¹⁶

### Objective

To determine the effects of long-acting GLP-1 receptor agonist, liraglutide, and placebo SQ over 16 weeks on weight and gastric functions and to evaluate associations of single nucleotide polymorphisms (SNPs) in GLP-1R (rs6923761) and TCF7L2 (rs7903146) with effects of liraglutide.

### Methods

### Study Design and Participants

A single-center (Mayo Clinic in Rochester, MN, USA), double-blind, placebo-controlled, parallel-group trial of once daily, SQ liraglutide 3 mg or placebo (1:1) for a total treatment period of 16 weeks was conducted. The pilot study results in the first 40 patients were published elsewhere.¹⁰

Adults with obesity (BMI>30 kg/m²), 18-65 years of age residing within 125 miles of the center were recruited. Participants were otherwise healthy, with no unstable psychiatric or medical disease or treatment that could interfere with the study conduct or interpretation. The study was approved by Mayo Clinic Institutional Review Board (IRB #15-001783). All participants provided written informed consent.

Patients with delayed gastric emptying of solids (>90th percentile according to gender, <87% in males or <81% emptied at 4 hours in females¹⁷) were excluded, since it was considered potentially dangerous to increase the delay in gastric emptying with a GLP-1 receptor agonist.

### Study Protocol

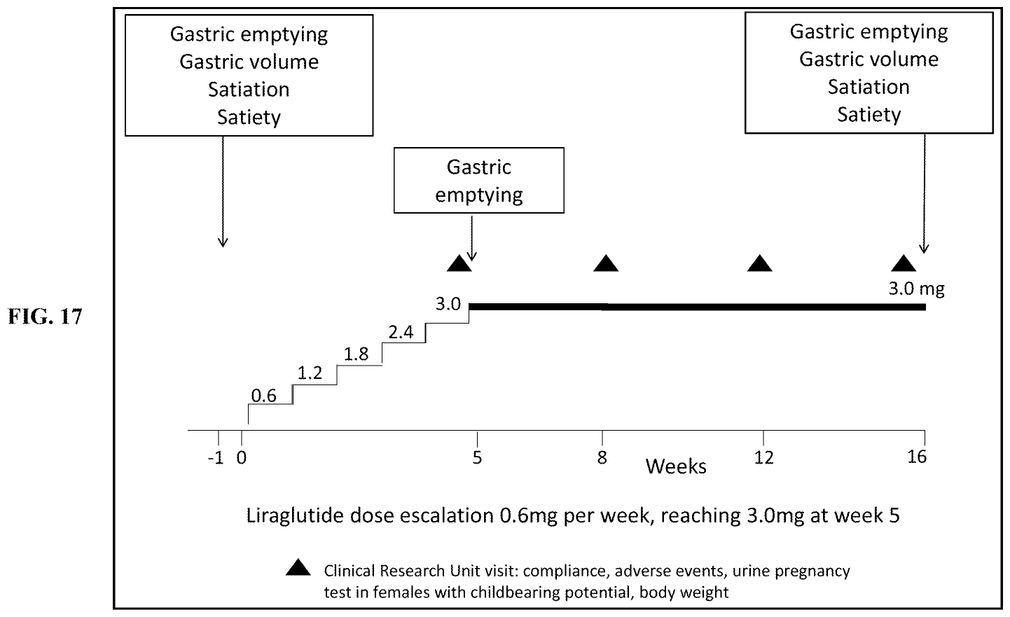

FIG. 17 shows the study protocol. All study participants underwent screening visits, baseline measurements of gastrointestinal, behavioral, and psychological factors, and dose escalation (0.6 mg per week for liraglutide, and similar weekly volume increments for placebo).

### Measurements of Gastrointestinal Functions

1. Gastric emptying of solids was assessed by scintigraphy using a 320 kcal ^{99m}Tc-radiolabeled egg, solid-liquid meal.¹⁷ The primary endpoint was gastric half-emptying time (GE T_{1/2}). GE of liquids is generally regarded to be a minor factor in the context of upper gastrointestinal symptoms;²¹ to reduce radiation burden, GE of solids was exclusively studied.

2. Fasting and postprandial gastric volumes were measured by single photon emission computed tomography (SPECT) imaging of the stomach after intravenous injection of ^{99m}Tc-pertechnetate, which is taken up by the gastric mucosa. This method was developed and validated (including performance characteristics) previously 22 and provides volume measurements during fasting and post-300 mL Ensure®.

3. Satiation test by ingestion of Ensure® (1 kcal/mL, 11% fat, 73% carbohydrate, and 16% protein) ingested at a constant rate of 30 ml/minute was performed to measure volume to fullness (VTF) and maximum tolerated volume (MTV).²³ Thirty minutes after reaching MTV, symptoms of fullness, nausea, bloating, and pain were measured using 100 mm horizontal visual analog scales (VAS), with the words “none” and “worst ever” anchored at each end.

4. Satiety test (a measure of appetite) by ad libitum meal measured total caloric intake and macronutrient distribution in the chosen foods from standard foods of known nutrient composition:¹ vegetable lasagna (Stouffers, Nestle USA, Inc., Solon, OH, USA]; vanilla pudding (Hunts, Kraft Foods North America, Tarrytown, NY, USA); and skim milk. The total kilocalories of food consumed and macronutrients ingested at the ad libitum meal were analyzed by validated software (ProNutra 3.0; Viocare Technologies Inc., Princeton, NJ, USA).

5. Plasma peptide YY (PYY) levels by radioimmunoassay were measured fasting, and 15, 45, and 90 minutes postprandially. PYY was measured by radioimmunoassay (Millipore Research, Inc. (St. Louis, MO) PYY exists in at least 2 molecular forms, 1-36 and 3-36, both of which are physiologically active and were detected by the assay.

### Measurement of Body Composition

Body composition was determined at baseline and at 16 weeks of treatment via dual-energy x-ray absorptiometry (DXA) technology using a Lunar iDXA (GE Healthcare, Madison, WI) as previously described.²⁴

A research support technician with Limited Scope X-ray Operator certification (State of MN) performed full body scans. Scans were analyzed with enCORE software (version 15.0; GE Healthcare). Participants wore light clothing and removed all metal jewelry and other materials that could interfere with the x-ray beam. Quality control was performed daily before scanning the first participant using a phantom. The study technician analyzed all scans in an identical manner and was blind to group allocation. The Lunar iDXA is equipped for visceral and subcutaneous fat measurement. Standard D×A regions of interest (ROI) including the upper body (android) and trunk regions (which are associated with risk of chronic disease), the lower body region (gynoid, prominent in women) and total body fat (TBF) were assessed. The trunk ROI included everything except the head, arms, and legs.

Quantitative traits (Table 5) of gastric emptying of solids (standardized 320-kcal solid-liquid meal¹⁷), satiation by ad libitum meal, volume to fullness, and maximum tolerated volume of liquid nutrient meal,²³ fasting and postprandial gastric volumes (in response to a standard volume of 300 mL Ensure®22), and body composition by DEXA²⁴ were measured at baseline and at week 16. An additional scintigraphic gastric emptying test with the same solid-liquid meal was performed at week 5.

TABLE 5
Baseline measurements in the two treatment groups that constitute ITT cohorts

| Data show median and IQR | Placebo | | Liraglutide | |
|---|---|---|---|---|
| Weight, kg | 100.0 | (92.4, 114.9) | 103.1 | (89.1, 111.9) |
| Total percent fat (%) | 47.9 | (43.5, 51.8) | 48.6 | (45.4, 51.1) |
| Trunk percent fat (%) | 51.8 | (46.6, 56.0) | 52.7 | (49.0, 54.9) |
| Fasting glucose, mg/dL | 94.0 | (86.0, 101.0) | 93.0 | (87.0, 102.0) |
| GE T_{25%}, min | 63.2 | (48.5, 75.0) | 66,4 | (55.7, 87.5) |
| GE T_{1/2}, min | 108.0 | (93.1, 128.6) | 117.2 | (97.5, 140.0) |
| Gastric fasting volume, mL | 200.8 | (179.3, 231.2) | 200.4 | (179.3, 231.2) |
| Gastric postprandial volume, mL | 587.0 | (525.4, 678.0) | 593.5 | (489.3, 648.6) |
| Gastric accommodation volume, mL | 378.5 | (322.5, 455.9) | 377.1 | (322.6, 445.3) |
| Satiation volume to fullness, mL | 756 | (535.5, 945.0) | 693 | (567.0, 871.0) |
| Satiation maximum tolerated volume, mL | 1244.3 | (995.4, 1493.1) | 1244.3 | (995.4, 1244.3) |
| VAS Aggregate Score (max 400) | 187.0 | (141.5, 253.5) | 217.0 | (116.0, 261.0) |
| ad libitum meal total calories | 878.6 | (708.2, 1151.1) | 829.5 | (665.7, 1088.5) |

| | | |
|---|---|---|
| GLPI rs6923761: % AG/AA, % GG | 42%/58% | 53%/47% |
| TCF7L2 rs7903146 > % CT/TT; % CC | 49.3%/50.7% | 40.9%/59.1% |

### Liraglutide

Liraglutide was administered as recommended by the FDA (www.accessdata.fda.gov/drugsatfda_docs/label/2014.pdf): initiated at 0.6 mg daily for one week, with instructions to increase by 0.6 mg weekly until 3.0 mg was reached (˜ over 4 weeks).

### Standardization of Dietetic and Behavioral Advice

Patients received standardized dietetic and behavioral advice for weight reduction therapy. All participants met with a behavioral psychologist or study coordinators who had expertise in obesity treatment at the baseline visit and at visits at weeks 4, 8 and 12. The behavioral interventionist followed a session outline to standardize session content. Study participants were taught a range of behavioral skills for successful weight management. These were brief (15 to 20 minutes), standardized counseling sessions that incorporated motivational interviewing strategies.

The interventionists then completed visit forms about the content of the completed counseling session. Additionally, study participants had brief (10 minute) contact with a member of the study team every 4 weeks to inquire about their adherence to study protocol, any difficulties they were experiencing, whether they were reading the educational assignments, and to answer any additional questions arising from their reading material. In addition, adherence to medication intake was assessed using a daily dosing diary and review of diaries with subjects at each visit, as shown in Table 6 for sessions 1 to 4.

The first 55 participants were given a standard text for information (“LEARN” Manual, 10^{th} ed.).¹ The remaining 81 participants were given a standard text for information, the Mayo Clinic Diet book.²

TABLE 6
Standardized Sessions of Brief Dietetic and Behavioral Counseling

| Session | Discussion and values | | Reading assigned |
|---|---|---|---|
| 1 | a. | written patient education materials: | chapters 1-4 of the |
| | | Introduce the LEARN manual or Mayo | LEARN manual or Mayo |
| | | Clinic Diet book; | Clinic Diet Book |
| | b. | discuss the concept of readiness for change; | Chapters 1-5 prior to |
| | c. | differences between lifestyle change and | next visit |
| | | diet highlighted; | |
| | d. | value of keeping food records, identifying | |
| | | eating triggers; | |
| | e. | importance of eating 3 scheduled meals per | |
| | | day | |
| 2 | a. | review chapters 1-4 of the LEARN manual | chapters 5-8 of the |
| | | or Mayo Clinic Diet Book Chapters 1-5; | LEARN manual; Mayo |
| | b. | eating triggers, the benefits of physical | Clinic Diet Book |
| | | activity, and problem-solving strategies | Chapters 6-10 prior to |
| | | | next visit |
| 3 | a. | review chapters 5-8 of the LEARN manual | chapters 9-12 of the |
| | | or Mayo Clinic Diet Book Chapters 6-10; | LEARN manual; Mayo |
| | b. | role of social support for health behavior | Clinic Diet Book |
| | | changes; | Chapters 11-15 prior to |
| | c. | strategies for goal setting and controlled | next visit |
| | | eating | |

| | | |
|---|---|---|
| 4 | a. | review chapters 9-12 of the LEARN manual or Mayo Clinic Diet Book |
| | | Chapters 11-15; |
| | b. | review progress in physical activity and meal planning; |
| | c. | strategies to challenge negative thinking and relapse prevention |
| | | techniques; |
| | d. | encourage participants to read Mayo Clinic Diet Book Chapters 16-20 |

### Genotyping

Genotyping was performed as previously reported.²⁵ Established PCR-based methods were used using TaqMan® SNP Genotyping Assays rs6923761 (GLP-1 [catalog no. C_25615272_20]) and rs7903146 (TCF7L2 [catalog no. C_29347861_10]; Applied Biosystems, Foster City, CA, USA) in accordance with the manufacturer's instructions. Following polymerase chain reaction amplification, end reactions were analyzed with an ABI ViiA-7 Real-Time PCR System using QuantStudio™ Real-Time PCR software (Applied Biosystems).

### Outcomes

Time to half gastric emptying of solids (GES T_{1/2}) was the primary endpoint for analysis during the 5- and 16-week treatment periods. Secondary endpoints were weight loss at week 5 and week 16, satiation by ad libitum meal, volume to fullness and maximum tolerated volume, fasting, postprandial, accommodation gastric volumes, postprandial plasma PYY levels at 16 weeks, and percent total body and trunk fat relative to whole body composition (on DEXA imaging).

### Statistical Analysis

The statistical analysis addressed the hypothesis that there was a treatment effect with liraglutide compared to placebo on the study endpoints, based on analysis of covariance. Data were provided as median (interquartile range). All available data from all randomized patients were used in the statistical analyses. In addition, data were imputed for the 12 participants who dropped out. For each missing data, the average value for all patients in the study was inputted and reduced the degrees of freedom by one for each data value imputed for that endpoint.

The effects of liraglutide and placebo were analyzed using analysis of covariance (ANCOVA), with the corresponding baseline measurement as a covariate, using an a of 0.05. The gastric emptying T_{1/2} of solids at 5 and 16 weeks in participants receiving liraglutide were compared using a paired t-test (test for normality passed using Shapiro-Wilk test). A dominant genetic model was used to assess the association of the two single nucleotide polymorphisms of interest in the GLP1R and TCF7L2 genes with phenotypes, especially weight, percent fat in body composition and gastric function.

Spearman correlations were used to assess the relationship between (absolute value of) gastric emptying Tin of solids at baseline, 5 and 16 weeks (as well as change between baseline and 5 or 16 weeks) and degree of weight loss on treatment. All analyses were conducted using SAS Version 9.4.

### Statistical Power

The present study with 65 patients in each treatment arm had 80% power (at α=0.05) to detect a difference in absolute gastric emptying T_{1/2} of 14.8 minutes between the treatment groups based on GE Tin mean±SD of 121.7±29.8 minutes published previously¹⁶ from a study of 319 healthy human volunteers. Effect sizes demonstrable for weight loss and other quantitative traits are shown in Table 7.

TABLE 7
Power calculations: Effect sizes demonstrable for primary and
secondary endpoints based on comparison of 2 treatment groups
(liraglutide vs. placebo) with 65 patients per treatment group (data
based on prior studies³ using same validated methods in same
laboratory or published literature for weight loss)

| | | | Effect size detectable | |
|---|---|---|---|---|
| Response | Mean | SD | [absolute # (% of mean)] | |
| Weight loss over 16 weeks | 5.0 | 1.8 | 0.89 kg | (17.8%) |
| Gastric emptying solids, T_{1/2} | 121.7 | 29.8 | 14.8 min | (12.2%) |
| min | | | | |
| Fasting gastric volume (vol.), | 225 | 65 | 32.2 mL | (14.3%) |
| ml | | | | |
| Gastric accommodation vol., | 507 | 100 | 49.5 mL | (9.8%) |
| ml | | | | |
| ad libitum meal intake, kcal | 928 | 360 | 178 kcal | (19.2%) |
| Volume to fullness, ml | 755 | 330 | 163.5 mL | (21.7%) |
| Maximum tolerated volume, | 1283 | 400 | 198 mL | (15.4%) |

mL

### Results

### Study Evolution

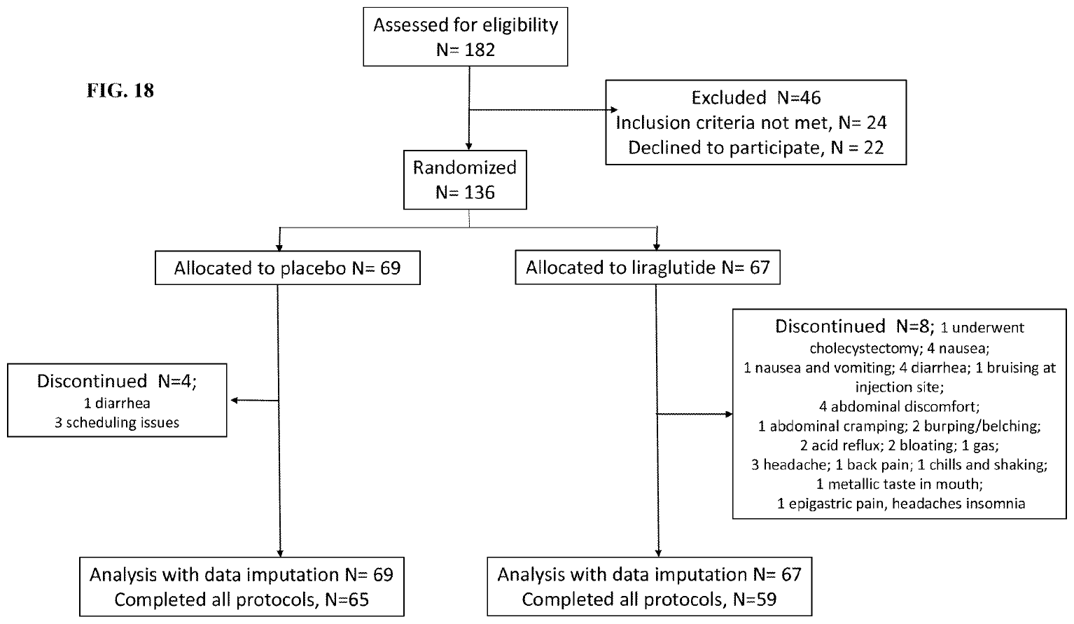

FIG. 18 shows the CONSORT flow chart with 182 adults assessed for eligibility, 136 randomized, and 124 completing the 16-week treatment trials (65 placebo and 59 liraglutide). Two participants did not reach full liraglutide dose at 16 weeks because of adverse effects (final doses 1.2 and 1.8 mg).

The baseline demographics and measurements in the two treatment groups were not significantly different (Tables 5 and 8). The lowest BMI at baseline was 30.09 kg/m². The median baseline GES T_{1/2} for the 136 participants was 113.6 minutes (10-90% ile 86.4, 148.9), which is consistent with the reported range for normal controls, median 120 minutes (10-90th % ile, 88, 163).¹⁷ Participants had no co-morbidities, except for one who had type 2 diabetes (T2DM) at enrollment; a second participant was diagnosed with T2DM during the study and was treated with metformin. The distributions of alleles for the entire group were as follows: GLP1R rs6923761: 64 (47%) AG/AA, and 71 (53%) GG; and TCF7L2 rs7903146: 61 (45%) CT/TT and 74 (55%) CC; the allelic distributions between the two treatment groups (Table 5) were not significantly different: GLP1R rs6923761 (p=0.200) and TCF7L2 rs7903146 (p=0.329).

TABLE 8
Effects of liraglutide, 3.0 mg, on gastric emptying and weight after
5 weeks' and 16 weeks' treatment (based on ITT population and
P values based on rank sum test). Data show absolute values and delta
variables which were calculated as Week 5 or Week 16, minus baseline.

| | | | |
|---|---|---|---|
| | Placebo, | Liraglutide, | Overall |
| Data show median and IQR | n = 69 | n = 67 | p* |

Demographic features at baseline

| N randomized | 69 | | 67 | |
|---|---|---|---|---|
| Age, y | 37.2 | (29.3, 45.2) | 42 | (32, 51) |
| Sex (% female) | 85.5% | | 88.1% | |
| Race, % white | 94.2% | | 89.6% | |
| BMI, kg/m² | 35.6 | (33.1, 39.7) | 35.9 | (32.6, 40.2) |

Body weight (kg) and percent fat

| | | | | | |
|---|---|---|---|---|---|
| Baseline weight, kg | 100.0 | (92.4, 114.9) | 103.1 | (89.1, 111.9) | |
| Weight @ 5 weeks, kg | 101.4 | (90.5, 114.2) | 100.4 | (87.0, 108.6) | |
| Weight @ 16 weeks. kg | 99.0 | (90.8, 114.6) | 97.9 | (85.9, 108.3) | |
| Delta Weight @ 5 weeks vs. baseline | 0.1 | (−1.5, 1.4) | −3.8 | (−4.8, −2.5) | 0.004 |
| Delta Weight @ 16 weeks vs. baseline | 0.0 | (−3.1, 2.1) | −5.8 | (−8.3, −3.9) | 0.033 |
| Baseline total percent fat (%) | 47.9 | (43.5, 51.8) | 48.6 | (45.4, 51.1) | |
| 16 weeks % total fat | 47.6 | (42.6, 52.0) | 47.3 | (43.7, 49.1) | |
| Delta % total fat @ 16 weeks vs. | −0.5 | (0.9, −1.3) | −2.0 | (−0.9, −3.1) | 0.008 |
| baseline | | | | | |
| Baseline % trunk fat | 51.5 | (46.6,56.0) | 52.7 | (49.0, 54.9) | |
| 16 weeks % trunk fat | 50.8 | (45.6, 55.7) | 49.7 | (46.3, 53.8) | |
| Delta % trunk fat @ 16 weeks vs. | −0.9 | (−1.8, 0.9) | −2.5 | (−4.0, −1.0) | 0.004 |
| baseline | | | | | |

Gastric emptying, min

| | | | | | |
|---|---|---|---|---|---|
| Baseline GES T25%, min | 63.2 | (48.5, 75.0) | 66,4 | (55.7, 87.5) | |
| GES T25% @ 5 weeks, min | 63.6 | (56.0, 80.0) | 117.2 | (75.0, 156.1) | |
| GES T25%, @ 16 weeks, min | 65.0 | (52.5, 83.1) | 85 | (59.7, 114.3) | |
| Delta GES T25% @ 5 weeks vs. | 1 | (−10.2, 14.2) | 44.8 | (4.1, 94.1) | <0.001 |
| baseline, min | | | | | |
| Delta GES T25% @ 16 weeks vs. | 0.7 | (13.3, −10.0) | 13.2 | (48.4, −7.2) | 0.011 |
| baseline, min | | | | | |
| Baseline GES T_{1/2}, min | 108.0 | (93.1, 128.6) | 117.2 | (97.5, 140.0) | |
| GES T_{1/2} @ 5 weeks, min | 105.9 | (92.6, 127.8) | 191.6 | (137.0, 241.0) | |
| GES T_{1/2} @ 16 weeks, min | 111.4 | (97.3, 132.9) | 154.4 | (120.4, 178.3) | |
| Delta GES T_{1/2} @ 5 weeks vs. baseline, | −0.1 | (−14.4, 16.4) | 69.7 | (32.3, 97.1) | <0.001 |
| min | | | | | |
| Delta GES T_{1/2} @ 16 weeks vs. | 1.8 | (−11.2, 14.1) | 33.8 | (3.7, 63.4) | <0.001 |
| baseline, min | | | | | |

### Effects of Treatment on Gastrointestinal Motor Functions, Weight and Satiation

Data in the two treatment groups are shown in Tables 8 and 9, demonstrating the significant effects of liraglutide on GES T_{1/2} and weight documented by changes from baseline values.

TABLE 9
Effects of liraglutide, 3.0 mg, on gastric accommodation, satiation, and satiety (B) after 5 weeks'
and 16 weeks' treatment (based on ITT population and P values based on rank sum test). Data
show absolute values and delta variables which were calculated as Week 5 or Week 16, minus baseline.

| | | | |
|---|---|---|---|
| | Placebo, | Liraglutide, | Overall |
| Data show median and IQR | n = 69 | n = 67 | p# |

Gastric emptying volume, mL

| | | | | | |
|---|---|---|---|---|---|
| Baseline gastric fasting volume, mL | 200.8 | (179.3, 231.2) | 200.4 | (179.3, 231.2) | |
| Baseline gastric postprandial vol., mL | 587.0 | (525.4, 678.0) | 593.5 | (489.3, 648.6) | |
| Baseline gastric accommodation vol, mL | 378.5 | (322.5, 455.9) | 377.1 | (322.6, 445.3) | |
| Gastric fasting volume @16 weeks, mL | 191.5 | (176.5, 231.5) | 221.2 | (187.7, 269.8) | |
| Gastric postprandial vol @16 weeks, mL | 583.8 | (549.8, 667.7) | 629.1 | (538.9, 705.1) | |
| Gastric accommodation (accomm.) vol. @16 | 391.8 | (348.6, 433.5) | 385.4 | (332.6, 445.2) | |
| weeks, mL | | | | | |
| Delta Gastric fasting volume @ 16 weeks vs. | −5.9 | (−39.9, 24.7) | 30.0 | (−24.1, 77.6) | 0.010 |
| baseline | | | | | |
| Delta Gastric postprandial vol @ 16 weeks | −6.7 | (−67.0, 78.6) | 50.1 | (−45.2, 126.8) | 0.14 |
| vs. baseline | | | | | |
| Delta Gastric accomm. vol @ 16 weeks vs. | 4.7 | (−54.7, 86.2) | 9.2 | (−58.7, 87.7) | 0.73 |
| baseline | | | | | |

Satiation volume (mL) and symptoms (VAS, mm)

| | | | | | |
|---|---|---|---|---|---|
| Baseline satiation volume to fullness (VTF), | 756 | (535.5, 945.0) | 693 | (567.0, 871.0) | |
| mL | | | | | |
| Baseline satiation maximum tolerated | 1244.3 | (995.4, 1493.1) | 1244.3 | (995.4, 1244.3) | |
| (MTV), mL | | | | | |
| Satiation VTF @16 weeks, mL | 746.6 | (497.7, 871.0) | 622.1 | (496.7, 746.6) | |
| Satiation MTV @16 weeks, mL | 1119.8 | (995.4, 1430.9) | 974.4 | (746.6, 1156.3) | |
| Delta, satiation VTF (mL), @16 weeks vs. | 0.0 | (−126.0, 124.4) | −124.4 | (−248.9, 41.0) | 0.006 |
| baseline | | | | | |
| Delta, Satiation MTV (mL) @16 weeks vs. | −124.4 | (−248.9, 85.3) | −248.9 | (−497.7, 0.0) | <0.001 |
| baseline | | | | | |
| Baseline VAS aggregate score | 206.0 | (151.5, 256.5) | 204.0 | (156.0, 253.0) | |
| Baseline VAS nausea score | 33.5 | (19, 64) | 43 | (12, 62) | |
| Baseline VAS fullness score | 77.5 | (72.5, 83) | 74 | (66, 84) | |
| Baseline VAS bloating score | 66.5 | (49, 78.5) | 67 | (52, 79) | |
| Baseline VAS pain score | 27.5 | (7.5, 50) | 27 | (10, 55) | |
| VAS aggregate score @16 weeks | 219.5 | (179.5, 258.5) | 236.0 | (188, 277) | |
| VAS nausea score @16 weeks | 37.0 | (23.5, 61) | 48 | (21, 66) | |
| VAS fullness score @16 weeks | 75 | (70, 82.5) | 74 | (68, 81) | |
| VAS bloating score @16 weeks | 71 | (53, 82.5) | 74 | (55, 81) | |
| VAS pain score @16 weeks | 29 | (14.5, 59.5) | 51 | (21, 63) | |
| Delta, VAS aggregate score @16 weeks vs. | 6.0 | (−20.0, 53.0) | 24.0 | (−34.0, 67.0) | 0.28 |
| baseline | | | | | |

Satiety (appetite), kcal ingested

| | | | | | |
|---|---|---|---|---|---|
| Baseline ad libitum meal total calories | 878.6 | (708.2, 1151.1) | 829.5 | (665.7, 1088.5) | |
| ad libitum meal total calories at 16 weeks | 793.7 | (624.6, 1019.3) | 647.5 | (472.4, 826.4) | |
| Delta ad libitum meal total calories at 16 | −129.2 | (−197.6, −23.2) | −184.8 | (−322.3, −69.4) | 0.004 |
| weeks vs. baseline | | | | | |

#Analyses run using analysis of covariance (ANCOVA) model of rank transformed data; model covariates include baseline of dependent variable in the model, sex, and treatment arm.
Maximum tolerated volume (MTV); aggregate symptom score maximum is 400; individual symptom scores maximum 100.

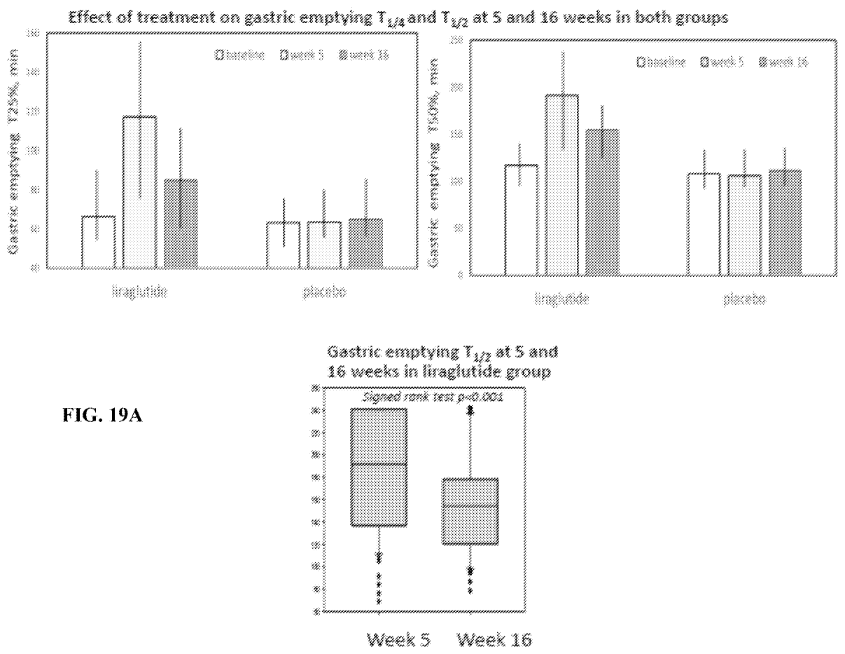

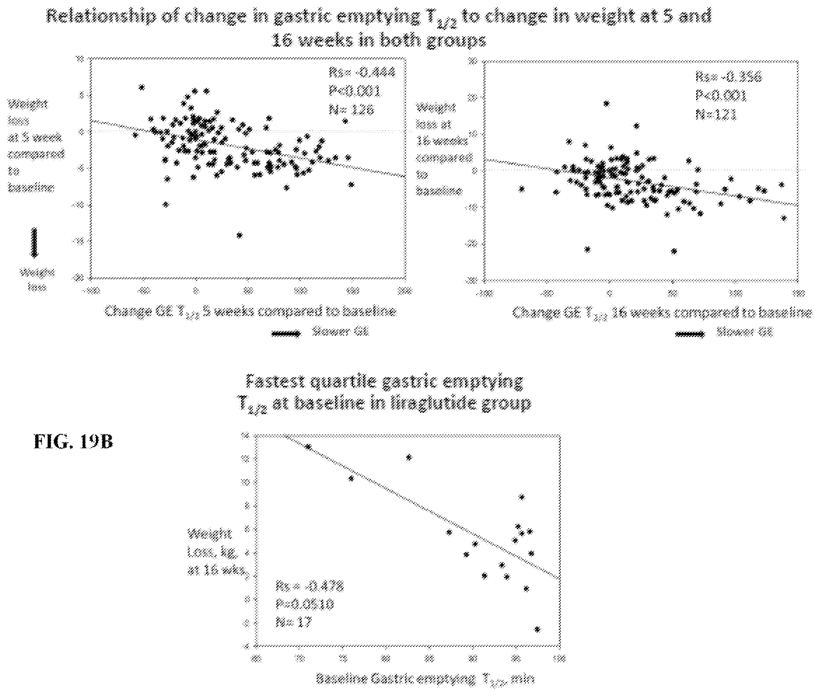

Liraglutide also prolonged (FIG. 19A, Table 8) times for 50% and 25% gastric emptying compared to placebo. In the liraglutide-treated group, GES T_{1/2} at 16 weeks was not as slow as at 5 weeks; thus, the delta of GES T_{1/2} at 16 weeks minus GES T_{1/2} at 5 weeks was −12.9 (IQR −62.7, 8.0) minutes (p<0.001).

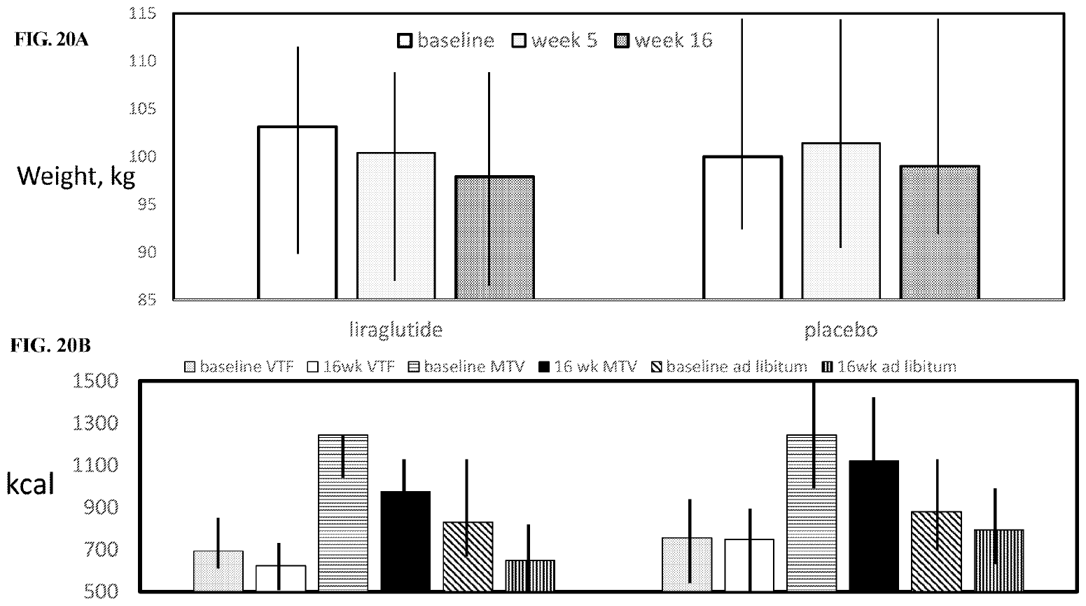

Weight loss (FIG. 20A, Table 8) was significantly greater for the liraglutide group compared to the placebo group at 5 weeks (p=0.004) and at 16 weeks (p=0.033).

There were significant effects of liraglutide on fasting gastric volumes at 16 weeks which was significantly higher (p=0.01) in the liraglutide group compared to the placebo group (Table 9); these were documented by comparison of the changes from baseline. The numerical difference in postprandial gastric volume noted in the liraglutide compared to the placebo group was not significant (p=0.14).

The volume to comfortable fullness (p=0.0056) and maximum tolerated volume (p<0.001) at 16 weeks were significantly lower in the liraglutide group compared to the placebo group (FIG. 20B, and Table 9), as documented by the changes from baseline. Postprandial symptoms after the satiation drink test were not significantly different in the two treatment groups.

There was also a significant difference (p=0.0036) in the calories consumed during an ad libitum meal in the group treated with liraglutide compared to placebo (FIG. 20B, and Table 9).

There were no significant effects of liraglutide on fasting and postprandial peptide YY (Table 10).

TABLE 10
Fasting and postprandial PYY levels (pg/mL) at baseline
and post-treatment (placebo or liraglutide)

| | Placebo | | Liraglutide | | |
|---|---|---|---|---|---|
| PYY | Baseline | On Rx | Baseline | On Rx | P |
| Fasting | 80 | 88 | 77.5 | 85.5 | NS |
| | (70.5, 111.0) | (75, 107) | (69, 104) | (70, 104) | |
| Post- | 122.7 | 123 | 111.2 | 120.7 | NS |
| prandial | (90, 154.7) | (100.7, 149.3) | (88.7, 154) | (89, 146) | |

### Relationship Between Gastric Emptying and Effect of Liraglutide on Weight Loss

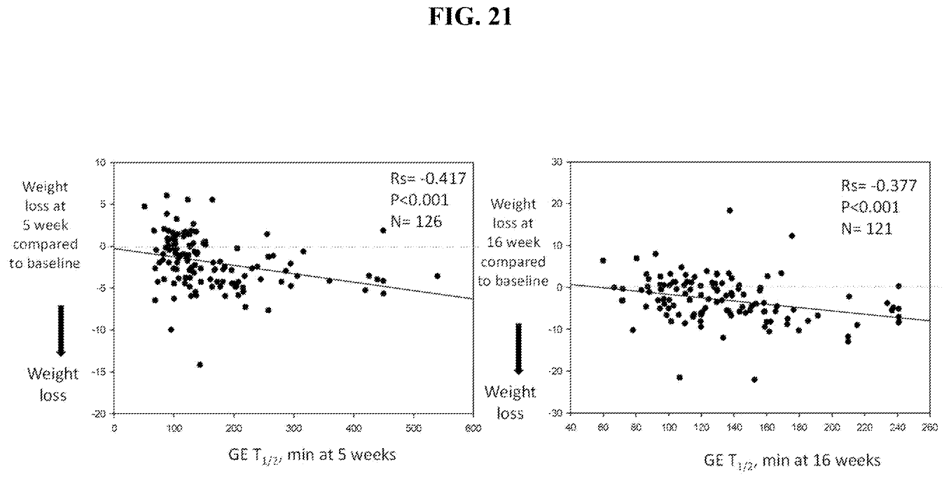

For the entire study cohort, there were significant correlations between GES T_{1/2} and weight loss, particularly between change in GES T_{1/2} at 5 weeks and 16 weeks (FIG. 19B) and the weight loss (expressed as delta from baseline) over the 5- and 16-week periods (FIG. 20A) (all p<0.001). FIG. 21 shows the significant Spearman correlations for the associations of GES T_{1/2} at 5 and 16 weeks and weight loss with treatment in the two groups (both P<0.001).

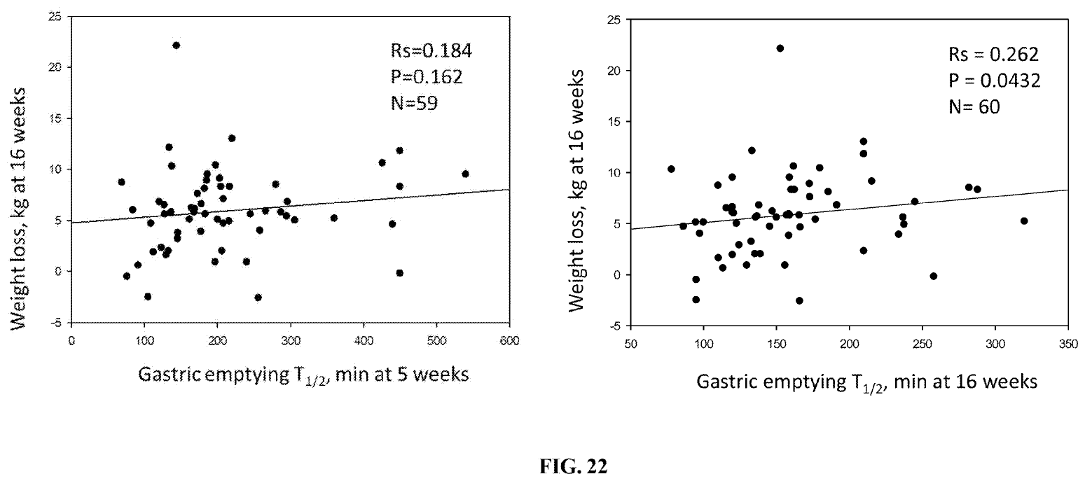

Moreover, in the liraglutide treatment group alone, there was significant direct correlation of GES T_{1/2} at 16 weeks and weight loss over the 16-week period (Rs=0.262, p=0.0432, N=60), but no significant correlation at 5 weeks (FIG. 22).

There was borderline significant correlation between the fastest quartile of GES T_{1/2} at baseline (≤97.5 min) and weight loss in response to liraglutide at week 5 (Rs=−0.432; P=0.081; N=17) and at week 16 (Rs==0.478; P=0.051; N=17) (FIG. 19B). In addition, after adjusting for baseline weight, total % fat, and trunk % fat, the fastest quartile of baseline GES T_{1/2} was associated with numerically lower percent total body and percent trunk fat after treatment with liraglutide for 16 weeks (respectively p=0.059 and 0.057 based on rank scores).

### Pharmacogenomics

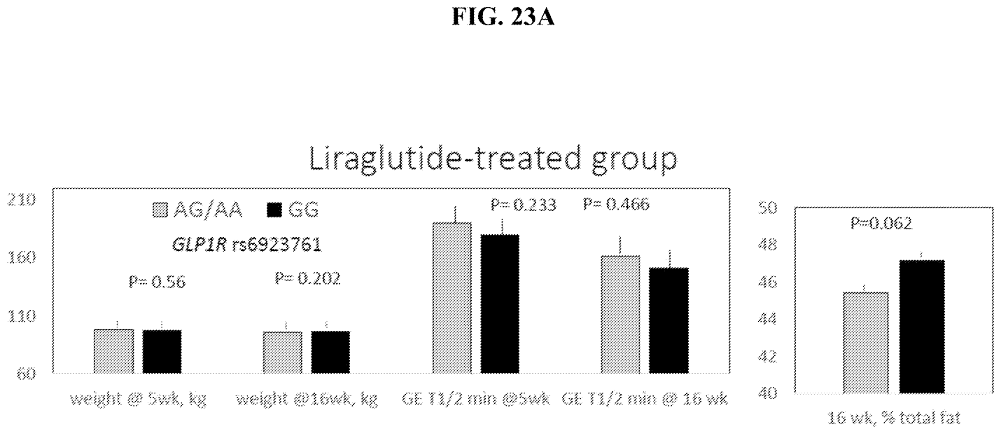

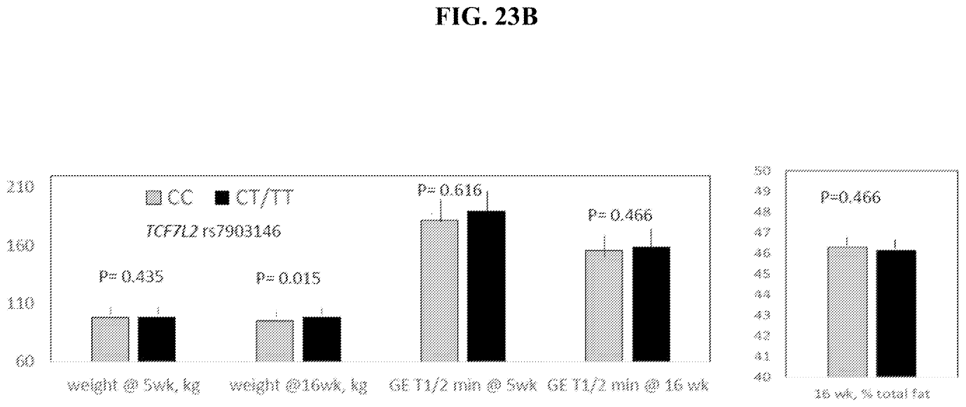

Based on a dominant genetic model to assess the association of the two single nucleotide polymorphisms of interest (FIGS. 23A-23B) in the GLP1R and TCF7L2 genes, GLP1R rs6923761 AG/AA genotype was associated with a lower % total fat in response to liraglutide (p=0.062; FIG. 23A). In addition, TCF7L2 rs7903146 CC genotype was associated with lower weight at 16 weeks in response to liraglutide compared to the CT/TT genotype (p=0.015; FIG. 23B). No other significant associations were identified between gene SNPs and other measurements.

In summary, liraglutide (n=59) and placebo (n=65) groups completed treatment. Relative to placebo, liraglutide increased weight loss at 5 and 16 weeks (both p<0.05), slowed GES T_{1/2} at 5 and 16 weeks (both p<0.001), increased fasting GV (p=0.01) and satiation (p<0.01)] at 16 weeks. GES T_{1/2} was positively correlated with weight loss on liraglutide (both p<0.001). After 16 weeks of liraglutide, GLP-1R rs6923761 (AG/AA vs. GG) was associated with reduced percentage body fat (p=0.062), and TCF71.2 rs7903146 (CC vs. CT/TT) with lower body weight (p=0.015).

### Conclusions

The randomized, controlled trial described herein has documented important phenotypic and genotypic mechanisms in the effects of 3 mg liraglutide on weight loss: retardation of gastric emptying of solids for at least 16 weeks of treatment and correlation of degree of weight loss with the retardation of gastric emptying of solids. Liraglutide also influenced appetite regulation and highlighted the association of clinically relevant endpoints (weight and percent body fat) and allelic variation in genes relevant to GLP-1.

The absolute and the change from baseline GES T_{1/2} were associated with the degree of weight loss during the first 5-week and the entire 16-week periods of liraglutide treatment. The significant correlation between degree of retardation of gastric emptying and weight loss is consistent with a mechanistic role of the gastric emptying effect on weight loss. Indeed, among the participants with obesity randomized to liraglutide, the quartile with the fastest gastric emptying at baseline showed correlation with the degree of weight lost, suggesting that baseline gastric motor function phenotype can play a role in a patient-tailored approach to obesity management. Similarly, tolerance of GLP-1 agonists or analogs can influence patient adherence and thus the effectiveness of these medications. Individuals with markedly delayed gastric emptying from study enrollment were excluded, and it was observed that experience of nausea was associated with greater weight loss, as has been previously documented in trials using with exenatide once weekly and exenatide twice daily.³⁰

It is interesting to note that, in large multicenter studies of liraglutide,^{31,32} approximately 50% of the average weight loss was achieved in the first 8 weeks of treatment, which included the period with the greatest delay in gastric emptying of solids in this study. While GES Tin correlated with concurrent weight loss at both 5 and 16 weeks, there was reduced effect on GES T_{1/2} with liraglutide at 16 weeks compared to 5 weeks. This is consistent with tachyphylaxis in the effect on gastric emptying of solids as previously described with GLP-1.³³ This phenomenon reflects continuous activation of the GLP-1 receptor by the long-acting GLP-1 receptor agonist, leading to tolerance.^{3,33} Nevertheless, there was still significant delay in GES T_{1/2} at 16 weeks, and weight loss continued from 5 to 16 weeks, suggesting a durable treatment effect from liraglutide even with diminished perturbation in gastric motor functions related to appetite.

Liraglutide also increased fasting gastric volume which is consistent with pharmacological effects of GLP-1,¹² but the postprandial gastric volume was not significantly increased. Importantly, the kilocalorie intake of and liquid nutrient at a standard rate (30 mL/min) and in an ad libitum meal were reduced by liraglutide, suggesting increased satiation without significant effect on postprandial levels of the appetite-modifying incretin, peptide YY. These observations may result from multiple mechanisms including delay in gastric emptying and activation of brainstem or hypothalamic GLP-1 receptors⁶ and central appetite suppression in the absence of increased postprandial gastric volume.

This study also provides the observation that SNPs impacting GLP-1R and TCF712 are associated with percent body and trunk fat and weight responses to liraglutide treatment. These data suggest that baseline accelerated gastric emptying and these loci may serve as biomarkers of weight loss (carriers of TCF71.2 SNP) and possibly the effect on total fat percentage (carriers of GLP-1R SNP). The TCF71.2 gene variant may impact the synthesis of endogenous GLP-1⁴¹⁻⁴⁴ and could impact the combined effects of the endogenous and exogenous GLP-1 receptor agonists.

In conclusion, the findings from this randomized clinical trial suggest that gastric emptying modulation, in addition to other central effects that are well-established, plays a role in weight loss with liraglutide, especially early in the treatment course. The correlation coefficients suggest that delay in gastric emptying accounts for about 20% of the variance (based on R²) in the weight loss response, and therefore, gastric emptying is certainly not the only mechanism contributing to weight loss effects. Moreover, baseline acceleration of gastric emptying appears to predict some of the variance in the weight loss on liraglutide. Effects on calorie intake are consistent with central effects of the drug or satiation associated with delayed gastric emptying,⁴⁵ and the pharmacogenetic observations described herein especially in TCF7L2 suggest biological genetic variation may also influence weight loss with liraglutide treatment. With further support in larger studies, these observations support potential individualized approaches for selection of patients for treatment of obesity with liraglutide.

In summary, liraglutide, 3 mg, induces weight loss with delay in GES T_{1/2} and reduces calorie intake. Slowing GES and variations in GLP-1R and TCF7L2 are associated with liraglutide effects in obesity.

### REFERENCES

1. Acosta A, Camilleri M, Shin A, Vazquez-Roque M I, Iturrino J, Burton D, et al. Quantitative gastrointestinal and psychological traits associated with obesity and response to weight-loss therapy. Gastroenterology 2015; 148:537-546.e4.
2. Maselli D B, Camilleri M. Effects of GLP-1 and its analogs on gastric physiology in diabetes mellitus and obesity. Adv Exp Med Biol. 2021; 1307:171-192.
3. Umapathysivam M M, Lee M Y, Jones K L, Annink C E, Cousins C E, Trahair L G, et al. Comparative effects of prolonged and intermittent stimulation of the glucagon-like peptide 1 receptor on gastric emptying and glycemia. Diabetes 2014; 63:785-790.
4. Secher A, Jelsing J, Baquero A F, Hecksher-Sørensen J, Cowley M A, Dalbøge L S, et al. The arcuate nucleus mediates GLP-1 receptor agonist liraglutide-dependent weight loss. J Clin Invest. 2014; 124:4473-4488.
5. Coveleskie K, Kilpatrick L A, Gupta A, Stains J, Connolly L, Labus J S, et al. The effect of the GLP-1 analogue Exenatide on functional connectivity within an NTS-based network in women with and without obesity. Obes Sci Pract. 2017; 3:434-445.
6. Farr O M, Sofopoulos M, Tsoukas M A, Dincer F, Thakkar B, Sahin-Efe A, et al. GLP-1 receptors exist in the parietal cortex, hypothalamus and medulla of human brains and the GLP-1 analogue liraglutide alters brain activity related to highly desirable food cues in individuals with diabetes: a crossover, randomised, placebo-controlled trial. Diabetologia 2016; 59:954-965.
7. Ten Kulve J S, Veltman D J, van Bloemendaal L, Groot P F C, Ruhé H G, Barkhof F, et al. Endogenous GLP1 and GLP1 analogue alter CNS responses to palatable food consumption. J Endocrinol. 2016; 229:1-12.
8. Farr O M, Upadhyay J, Rutagengwa C, DiPrisco B, Ranta Z, Adra A, et al. Longer-term liraglutide administration at the highest dose approved for obesity increases reward-related orbitofrontal cortex activation in response to food cues: Implications for plateauing weight loss in response to anti-obesity therapies. Diabetes Obes Metab. 2019; 21:2459-2464.
9. Vosoughi K*, Atieh J*, Khanna L, Khoshbin K, Prokop L J, Davitkov P, et al. (* joint 1^{st} authors). Association of glucagon-like peptide 1 analogs and agonists administered for obesity with weight loss and adverse events: A systematic review and network meta-analysis. EClinicalMedicine 2021 Nov. 27; 42:101213.
10. Halawi H*, Khemani D*, Eckert D, O'Neill J, Kadouh H, Grothe K, et al. (*joint first authors). Effects of liraglutide on weight, satiation and gastric functions in obesity: randomized, placebo-controlled pilot trial. Lancet Gastroenterol Hepatol. 2017; 2:890-899.
11. Deane A M, Nguyen N Q, Stevens J E, Fraser R J, Holloway R H, Besanko L K, et al. Endogenous glucagon-like peptide-1 slows gastric emptying in healthy subjects, attenuating postprandial glycemia. J Clin Endocrinol Metab. 2010; 95:215-221.
12. Delgado-Aros S, Kim D Y, Burton D D, Thomforde G M, Stephens D, Brinkmann B H, et al. Effect of GLP-1 on gastric volume, emptying, maximum volume ingested, and postprandial symptoms in humans. Am J Physiol Gastrointest Liver Physiol. 2002; 282:G424-G431.
13. Sathananthan A, Dalla Man C, Micheletto F, Zinsmeister A R, Camilleri M, Giesler P D, et al. Common genetic variation in GLPIR and insulin secretion in response to exogenous GLP-1 in nondiabetic subjects: a pilot study. Diabetes Care 2010; 33:2074-2076.
14. de Luis D A, Diaz Soto G, Izaola O, Romero E. Evaluation of weight loss and metabolic changes in diabetic patients treated with liraglutide, effect of RS6923761 gene variant of glucagon-like peptide 1 receptor. J Diabetes Complications 2015; 29:595-598.
15. Florez J C, Jablonski K A, Bayley N, Pollin T I, de Bakker P I W, Shuldiner A R, et al. TCF7L2 polymorphisms and progression to diabetes in the Diabetes Prevention Program. N Engl J Med. 2006; 355:241-250.
16. Vazquez-Roque M I, Camilleri M, Vella A, Carlson P, Laugen J, Zinsmeister A R. Association of TCF7L2 allelic variations with gastric function, satiation, and GLP-1 levels. Clin Transl Sci. 2011; 4:183-187.
17. Camilleri M, Iturrino J, Bharucha A E, Burton D, Shin A, Jeong I-D, et al. Performance characteristics of scintigraphic measurement of gastric emptying of solids in healthy participants. Neurogastroenterol Motil. 2012; 24:1076-e562.
18. Zigmond A S, Snaith R P. The hospital anxiety and depression scale. Acta Psych Scand. 1983; 67:361-370.
19. Bush K, Kivlahan D R, McDonell M B, Fihn S D, Bradley K A. The AUDIT alcohol consumption questions (AUDIT-C): an effective brief screening test for problem drinking. Ambulatory Care Quality Improvement Project (ACQUIP). Alcohol Use Disorders Identification Test. Arch Intern Med. 1998; 158:1789-1795.
20. Yanovski S Z, Marcus M D, Wadden T A, Walsh B T. The Questionnaire on Eating and Weight Patterns-5: an updated screening instrument for binge eating disorder. Intl J Eating Disord. 2015; 48:259-261.
21. Sachdeva P, Malhotra N, Pathikonda M, Khayyam U, Fisher R S, Maurer A H, et al. Gastric emptying of solids and liquids for evaluation for gastroparesis. Dig Dis Sci. 2011; 56:1138-1146.
22. Bouras E P, Delgado-Aros S, Camilleri M, Castillo E J, Burton D D, Thomforde G M, et al. SPECT imaging of the stomach: comparison with barostat and effects of sex, age, body mass index, and fundoplication. Gut 2002; 51:781-786.
23. Chial H J, Camilleri C, Delgado-Aros S, Burton D, Thomforde G, Ferber I, et al. A nutrient drink test to assess maximum tolerated volume and postprandial symptoms: effects of gender, body mass index and age in health. Neurogastroenterol Motil. 2002; 14:249-253.
24. Kadouh H, Chedid V, Halawi H, Burton D D, Clark M M, Khemani D, et al. GLP-1 analog modulates appetite, taste preference, gut hormones and regional body fat stores in adults with obesity. J Clin Endocrinol Metab. 2020; 105:1552-1563.
25. Chedid V, Vijayvargiya P, Carlson P, Van Malderen K, Acosta A, Zinsmeister A, et al. Allelic variant in the glucagon-like peptide 1 receptor gene associated with greater effect of liraglutide and exenatide on gastric emptying: a pilot pharmacogenetics study. Neurogastroenterol Motil. 2018; 30:e13313.
26. van Can J, Sloth B, Jensen C B, Flint A, Blaak E E, Saris W H. Effects of the once-daily GLP-1 analog liraglutide on gastric emptying, glycemic parameters, appetite and energy metabolism in obese, non-diabetic adults. Intl J Obes. 2014; 38:784-793.
27. Frandsen C S, Dejgaard T F, Andersen H U, Holst J J, Hartmann B, Thorsteinsson B, et al. Liraglutide as adjunct to insulin treatment in type 1 diabetes does not interfere with glycaemic recovery or gastric emptying rate during hypoglycaemia: a randomised, placebo-controlled, double-blind, parallel-group study. Diabetes Obes Metab. 2017; 19:773-782.
28. Jelsing J, Vrang N, Hansen G, Raun K, Tang-Christensen M, Knudsen L B. Liraglutide: short-lived effect on gastric emptying—long lasting effects on body weight. Diabetes Obes Metab. 2012; 14:531-538.
29. Nagai Y, Hashimoto E, Oikawa R, Asai S, Terashima Y, Nakamura Y, et al. Differing effects of liraglutide on gastric emptying in Japanese patients with type 2 diabetes. Diabetes Obes Metab. 2014; 16:573-576.
30. Horowitz M, Aroda V R, Han J, Hardy E, Rayner C K. Upper and/or lower gastrointestinal adverse events with glucagon-like peptide-1 receptor agonists: Incidence and consequences. Diabetes Obes Metab. 2017; 19:672-681.
31. Pi-Sunyer X, Astrup A, Fujioka K, Greenway F, Halpern A, Krempf M, et al. A randomized, controlled trial of 3.0 mg of liraglutide in weight management. N Engl J Med. 2015; 373:11-22.
32. le Roux C W, Astrup A, Fujioka K, Greenway F, Lau D C W, Van Gaal L, et al. Three years of liraglutide versus placebo for type 2 diabetes risk reduction and weight management in individuals with prediabetes: a randomised, double-blind trial. Lancet 2017; 389:1399-1409.
33. Nauck M A, Kemmeries G, Holst J J, Meier J J. Rapid tachyphylaxis of the glucagon-like peptide 1-induced deceleration of gastric emptying in humans. Diabetes 2011; 60:1561-1565.
34. Dahl K, Brooks A, Almazedi F, Hoff S T, Boschini C, Baekdal T A. Oral semaglutide improves postprandial glucose and lipid metabolism, and delays gastric emptying, in subjects with type 2 diabetes. Diabetes Obes Metab. 2021; 23:1594-1603.
35. Jones K L, Rigda R S, Buttfield M D M, Hatzinikolas S. Pham H T, Marathe C S, et al Effects of lixisenatide on postprandial blood pressure, gastric emptying and glycaemia in healthy people and people with type 2 diabetes. Diabetes Obes. Metab. 2019; 21:1158-1167.
36. Jalleh R, Pham H, Marathe C S, Wu T, Buttfield M D, Hatzinikolas S, et al, Acute effects of lixisenatide on energy intake in healthy subjects and patients with type 2 diabetes: relationship to gastric emptying and intragastric distribution. Nutrients. 2020; 12:1962.
37. Marathe C S, Pham H, Wu T, Trahair L G, Rigda R S, Buttfield M D M, et al. Acute administration of the GLP-1 receptor agonist lixisenatide diminishes postprandial insulin secretion in healthy subjects but not in type 2 diabetes, associated with slowing of gastric emptying. Diabetes Ther. 2022 Apr. 22. doi: 10.1007/s13300-022-01258-4.
38. Urva S, Coskun T, Loghin C, Cui X, Beebe E, O'Farrell L, et al. The novel dual glucose-dependent insulinotropic polypeptide and glucagon-like peptide-1 (GLP-1) receptor agonist tirzepatide transiently delays gastric emptying similarly to selective long-acting GLP-1 receptor agonists Diabetes Obes Metab. 2020; 22:1886-1891.
39. Kuwata H, Yabe D, Murotani K, Fujiwara Y, Haraguchi T, Kubota S, et al. Effects of glucagon-like peptide-1 receptor agonists on secretions of insulin and glucagon and gastric emptying in Japanese individuals with type 2 diabetes: A prospective, observational study. J Diabetes Investi. 2021; 12:2162-2171.
40. www.ema.europa.eu/en/documents/scientific-conclusion/saxenda-h-c-psusa-00001892-201812-epar-scientific-conclusions-grounds-variation-terms-marketing_en.pdf (accessed Apr. 26, 2022)
41. Yi F, Brubaker P L, Jin T. TCF-4 mediates cell type-specific regulation of proglucagon gene expression by beta-catenin and glycogen synthase kinase-3beta. J Biol Chem. 2005; 280:1457-1464.
42. Grant S F A, Thorleifsson G, Reynisdottir I, Benediktsson R, Manolescu A, Sainz J, et al. Variant of transcription factor 7-like 2 (TCF7L2) gene confers risk of type 2 diabetes. Nat Genet. 2006; 38:320-323.
43. Shu L, Matveyenko A V, Kerr-Conte J, Cho J-H, McIntosh C H S, Maedler K. Decreased TCF7L2 protein levels in type 2 diabetes mellitus correlate with downregulation of GIP- and GLP-1 receptors and impaired beta-cell function. Hum Mol Genet. 2009; 18:2388-2399.
44. Le Bacquer O, Shu L, Marchand M, Neve B, Paroni F, Kerr Conte J, et al. TCF7L2 splice variants have distinct effects on beta-cell turnover and function. Hum Mol Genet. 2011; 20:1906-1915.
45. Halawi H, Camilleri M, Acosta A, Vazquez-Roque M, Oduyebo I, Burton D, et al. Relationship of gastric emptying or accommodation with satiation, satiety and postprandial symptoms in health. Am J Physiol Gastrointest Liver Physiol. 2017; 313:G442-G447.
46. Brownell K D. The LEARN® Program for Weight Control. Tenth Edition. American Health Publishing Company, 2004.
47. Hensrud D D. The Mayo Clinic Diet. Second Edition. Mayo Clinic Press, 2017.

### Example 7: Determining if Genetic Variants Associated with Accelerated Gastric Emptying in Patients with Obesity are Predictive of GLP-1 Responsiveness

### Objective

As a follow-up to the study described in Example 4, this Example describes an analysis of whether or not genetic variants in patients with obesity can be predictive of a said patient's responsiveness to treatment with GLP-1 or agonists thereof.

### Methods

In general, clinical study outcome data was used to select SNPs that suggest liraglutide response and then machine learning models were built to validate predictions of response using that genetic information. The clinical study outcome data came from a 60-sample liraglutide treatment arm of a placebo-controlled cohort. SNP-chip genotyping of 2.4 million SNPs from a venous blood sample collected at the start of the study sample was conducted for each subject in the treatment arm, along with said subject's weight at trial start and at 16 weeks. The week 16 weight of each subject was compared to the initial weight and a response to the treatment was marked if the total body weight loss was >=5%. It should be noted that in order to develop the predictive assay utilized in this Example, the 60-sample treatment arm was subdivided into a training set of 44 samples and a validation set of 16 samples. The SNPs were selected based on an analysis of the association between the SNP-chip genotype data and subject response to liraglutide. Selection also entailed a literature search to create an initial set of candidate set of 8 informative SNPs and 15 additional putatively informative SNPs, from which less-informative SNPs were computationally filtered out.

To validate the SNPs, a Lasso logistic regression models was constructed to predict liraglutide response from the SNPs' genotypes. Two cross-validation experiments and an independent validation were performed, measuring Area Under the receiver-operator characteristic Curve (AUC), sensitivity, specificity, and precision. For cross validation, 5-fold cross validation was performed 100 times and the mean of each statistic was recorded on the left-out fold. Cross validation was performed on the training data (44 patients) and the whole treatment group (60 patients). Independent validation was performed by training a single model on the 44 training samples and measuring the performance on the 16 independent samples.

### Results

As shown in Table 11, the SNP-based GLP-1 response predictor comprising the combined set of SNPs found in Table 11 predicted response to liraglutide with good sensitivity, specificity and precision.

TABLE 11
Statistics of the SNP-based GLP-1 response predictor (i.e., liraglutide).

| | | Cross Validated | | | | | | | | Independently Validated | | | |
|---|---|---|---|---|---|---|---|---|---|---|---|---|---|
| | Training | Discovery | | | | Full | | | | Full | | | |
| | Data | | Sensi- | Speci- | | | Sensi- | Speci- | | | Sensi- | Speci- | |
| Set | SNPs | AUC | tivity | ficity | Precision | AUC | tivity | ficity | Precision | AUC | tivity | ficity | Precision |
| Candidate | rs1047776, | 0.64 | 0.53 | 0.57 | 0.56 | 0.64 | 0.66 | 0.57 | 0.69 | 0.54 | 0.70 | 0.33 | 0.64 |
| SNP set 1 | rs17782313, | | | | | | | | | | | | |
| | rs3813929 | | | | | | | | | | | | |
| Table 3 | rs11118997, | 0.33 | 0.47 | 0.29 | 0.40 | 0.43 | 0.60 | 0.35 | 0.56 | 0.47 | 0.50 | 0.50 | 0.63 |
| SNPs | rs1664232, | | | | | | | | | | | | |
| | rs6923761, | | | | | | | | | | | | |
| | rs9342434, | | | | | | | | | | | | |
| | rs2335852, | | | | | | | | | | | | |
| | rs1885034, | | | | | | | | | | | | |
| | rs11020655 | | | | | | | | | | | | |
| | (as | | | | | | | | | | | | |
| | kgp339989) | | | | | | | | | | | | |
| Combined | rs11118997, | 0.52 | 0.49 | 0.52 | 0.53 | 0.57 | 0.59 | 0.48 | 0.62 | 0.64 | 0.70 | 0.67 | 0.78 |
| | rs1664232, | | | | | | | | | | | | |
| | rs6923761, | | | | | | | | | | | | |
| | rs9342434, | | | | | | | | | | | | |
| | rs2335852, | | | | | | | | | | | | |
| | rs1885034, | | | | | | | | | | | | |
| | rs1047776, | | | | | | | | | | | | |
| | rs17782313, | | | | | | | | | | | | |
| | rs3813929, | | | | | | | | | | | | |
| | rs11020655 | | | | | | | | | | | | |
| | (as | | | | | | | | | | | | |
| | kgp339989) | | | | | | | | | | | | |

### Example 8: Factors Associated with Successful Weight Loss in Obese Patients Treated with Liraglutide

### BACKGROUND

Obesity is a chronic and multifactorial disease, with a significant medical and economic burden. Semaglutide, a glucagon-like peptide-1 (GLP-1) receptor agonist, was recently approved for weight loss. Previous studies show that GLP-1 agonists delay gastric emptying (GE), possibly explaining its role in weight loss. The aim of this Example was to study the weight loss outcomes of semaglutide in patients with rapid GE compared to normal/slow GE.

### Methods

In this study, a retrospective data collection on the use of semaglutide in adults with overweight or obesity was performed. Patients with who used weekly semaglutide subcutaneous injections (up to 2.4 mg) for ≥3 months and had a GE scintigraphy test were included. The patients were divided into two groups: rapid and normal/slow GE. Rapid GE was defined as more than 55% of content emptied at 2 hours. The primary end point was comparing the total body weight loss percentage (TBWL %) in patients with rapid GE compared to normal/slow GE at 3 and 6 months. Continuous end points were analyzed using matched paired t test. Data are presented as mean±standard deviation.

### Results

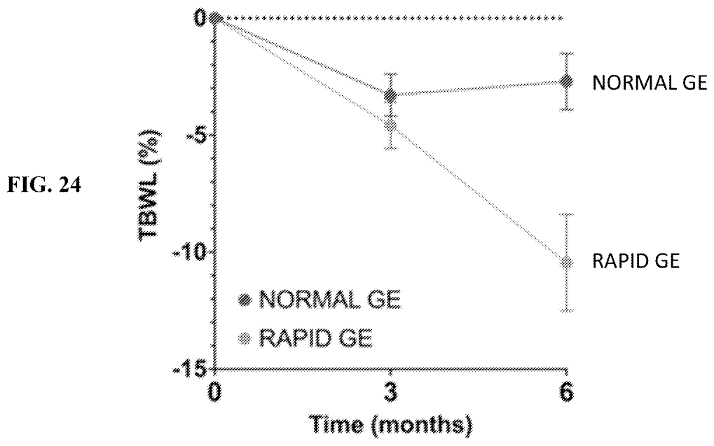

A total of 48 patients were included in the analysis (79% female, mean age 52±12.2 years, weight 102.2±25.9 kg, and 47% had diabetes). There were no differences in baseline demographics, anthropometrics, or prevalence of diabetes between groups (Table 12). There were 19 patients with rapid GE (GE120 min=72.2±16%) and 29 patients with normal/slow GE (GE120 min=35.8±11%). The TBWL % after 6 months was significantly lower among patients with rapid GE (n=14, −11.3±7.7) compare with normal/slow GE (n=19, −2.6±5.5) with a mean difference of 8.6% (95% CI 3.5-13.6; P=0.002) (FIG. 24).

### Conclusions

Semaglutide is associated with more weight loss in patients with rapid GE compared with patients with normal/slow GE. Gastric emptying might be an useful tool to predict weight loss response with semaglutide.

TABLE 12
Demographic and total body weight loss % of patients
with normal and rapid gastric emptying

| | Normal Gastric | Rapid Gastric | |
|---|---|---|---|
| Demographics | Emptying | Emptying | P-value |
| Participants, n | 29 | 19 | |
| Age, y | 51.7 ± 13.2 | 52.6 ± 10.8 | 0.80 |
| Sex, Female (%) | 23 (79%) | 15 (78%) | 0.97 |
| Weight, kg | 98.6.8 ± 24 | 107.8 ± 27 | 0.24 |
| BMI, kg/m² | 35.7 ± 9 | 38.3 ± 10 | 0.36 |
| Diabetes, yes (%) | 16 (55%) | 7 (36%) | 0.25 |
| Gastric emptying 1 hour, % | 22.4 ± 17 | 39.2 ± 26 | 0.02 |
| Gastric emptying 2 hour, % | 35.8 ± 11 | 72.2 ± 16 | <0.001 |
| Gastric emptying 4 hour, % | 73 ± 20 | 100 ± 24 | <0.001 |

Weight Loss Outcomes

| | | | |
|---|---|---|---|
| TBWL % 3 months (n = 43) | −3.3 ± 4.8 | −4.1 ± 3.5 | 0.53 |
| TBWL % 6 months (n = 33) | −2.6 ± 5.5 | −11.3 ± 7.7 | 0.002 |

### Example 9: Factors Associated with Successful Weight Loss in Obese Patients Treated with Liraglutide

### BACKGROUND

The response to non-surgical interventions for obesity such as diet, exercise, and pharmacotherapeutics remains highly variable and is often short lasting.¹⁻³ Liraglutide is a long-acting analog of human glucagon-like peptide-1 (GLP-1) that is approved by the United States Food and Drug Administration at a dosage of 3 mg per day administered subcutaneously (SQ) for weight management in adults with BMI≥30 kg/m2, or ≥27 kg/m2 with obesity related co-morbidities, and for pediatric population weighing at least 60 kg with BMI≥30 kg/m2 aged 12 years and older. It is proven effective in reducing weight in obese, non-diabetic individuals.⁴ Systematic reviews have shown that, as a class, GLP-1 agents are the most efficacious medications^{5, 6} and, among the GLP-1 analogs or agonists, the two most efficacious medications for inducing weight loss are SQ semaglutide < or >2.4 mg and SQ liraglutide >1.8 mg.⁷

Endogenous GLP-1, GLP-1 analogs, and GLP-1 receptor agonists induce weight loss through several peripheral and central mechanisms including delay of gastric emptying, activation of the ileal brake, increase in satiety, increase in resting energy expenditure, decrease in glucagon secretion, and direct modulation of appetite centers.⁸⁻¹⁴ While the principal mechanistic driver of weight loss is still unknown, it is established that there is no thermogenic effect of liraglutide, and therefore the dominant mechanism is considered to be related to caloric restriction rather than increased energy expenditure.¹⁵ Gastrointestinal functions and postprandial satiation may impact the variable outcomes of obesity therapy. As a pharmacological class, GLP 1 analogs or agonists significantly retard gastric emptying.⁸

In a trial involving 136 participants (initial pilot data and full data published),^{16, 17} 3 mg liraglutide significantly delayed time to half gastric emptying of solids (GET1/2) at 5 and 16 weeks, with the delay in gastric emptying being significantly correlated with weight loss at 6 weeks.^{16, 17} Given that exogenous GLP-1 is known to increase both fasting and postprandial gastric volumes,¹⁸ it is was hypothesized that liraglutide also affects gastric volumes and this may contribute to weight loss by altering appetite.

Energy intake has been used as a clinical measure to assess the effect of liraglutide on weight loss.^{11,14,19} Weight loss was found to be associated with a decrease in kcal consumed during ad libitum meal in two studies.^{14,19} A randomized, placebo-controlled trial of 3 mg liraglutide reported that calorie intake in a single ad libitum meal correlated with weight loss in nondiabetic patients with obesity.²⁰ However, there was no correlation with gastric emptying measured using plasma acetaminophen levels. Thus, it is still unclear what are the predictor(s) of weight loss in patients receiving liraglutide. Objective

It view of the foregoing, the hypothesis examined in this Example was that measurements of gastric functions such as gastric volumes, gastric emptying, plasma incretin levels, and satiety predict or are associated with weight loss at 16 weeks in response to 3 mg liraglutide SQ administered for 16 weeks. Therefore, the objective of this analysis was to identify the best predictors or factors associated with weight loss >4 kg among demographic parameters and gastrointestinal functions in obesity in response to 3 mg of SQ liraglutide administered for 16 weeks. The >4 kg weight loss was selected as a clinically relevant degree of loss over 16 weeks, given that the weighted mean difference in nine clinical trials of liraglutide >1.8 mg was 4.49 kg (3.72 to 5.26) when administered for mean 42.2 weeks (range 12-160 weeks).

### Methods

This single-center (Mayo Clinic, Rochester, MN, USA), double-blind, placebo-controlled, parallel-group trial of once daily SQ liraglutide 3 mg or placebo (1:1) for a total treatment period of 16 weeks has been published elsewhere.¹⁷ The current analysis to identify the best predictors or factors associated with weight loss was conducted based on the information collected including demographic parameters and gastrointestinal functions as detailed below.

Adults with obesity (BMI>30 kg/m2) who were otherwise healthy, 18-65 years of age, and residing within 125 miles of the center were recruited. Participants with delayed gastric emptying of solids (>90th percentile according to gender, <87% in males or <81% emptied at 4 hours in females)²¹ were excluded to ensure participant safety.

A total of 136 participants were enrolled up to May 1, 2021 and completed the studies by Aug. 31, 2021. Liraglutide was escalated as recommended by the FDA: 0.6 mg daily for one week and increased by 0.6 mg weekly increments until 3.0 mg was reached over 4 weeks. Every 4 weeks, participants obtained a new supply of study medication from the Research Pharmacy. Participants in both treatment groups received standardized dietetic and behavioral counseling for weight reduction therapy.¹⁷

All study participants underwent screening visits, baseline measurements of gastrointestinal, behavioral, and psychological factors, and dose escalation (0.6 mg per week for liraglutide, and similar weekly volume increments for placebo). Quantitative traits¹⁶ were measured as follows at baseline and after 16 weeks of treatment: gastric emptying of solids (standardized 320-kcal solid-liquid meal²¹), satiation by ad libitum meal, volume to fullness and maximum tolerated volume of liquid nutrient meal,²⁵ fasting and postprandial gastric volumes (in response to a standard volume of 300 mL Ensure®²⁶). An additional scintigraphic gastric emptying test with the same solid-liquid meal was performed at week 5. On the day of the nutrient drink test, participants had blood samples drawn fasting, and at 15, 45, and 90 minutes during and after nutrient drink ingestion to measure the incretin peptide YY (peptide tyrosine-tyrosine) which was quantified using the Human Peptide YY Double Antibody Radioimmunoassay Kit (Millipore Research, Inc., St. Louis, MO, USA). PYY exists in two or more molecular forms, 1-36 and 3-36, both of which are physiologically active and are detected by the assay.

All participants also had genotyping¹⁷ for TCF7L2 rs6923761 (AG/AA and GG) and GLP-1R rs7903146, which respectively modify the synthesis of endogenous GLP-1 and the functions of GLP-1 receptors respectively.

A multiple variable regression model was used to examine the likelihood of weight loss >4 kg in all patients and in patients in the liraglutide arm at 16 weeks of the study. A parsimonious model was fit using backward selection to identify the final model. Statistical analyses were performed using SAS Software, version 9.4 (SAS Institute). Odds ratios and corresponding 95% confidence intervals were calculated. All odds ratios for GET1/2 are reported for 10 minutes and 50 minutes of change in Table 14. The rationale for expressing the odds ratio for 50 minutes of change in GET1/2 was based on the observations of the effects of liraglutide in the parent study¹⁷ which was a median slowing at 5 weeks of 69.7 minutes (IQR: 32.3-97.1), and a median slowing at 16 weeks of 33.8 minutes (IQR:3.7-63.4). Odds ratios for ad libitum buffet meal are reported per 100 calories of change to better grasp the magnitude of the effect. All p-values that were lower than <0.001 were reported as p<0.001.

### Results

### Baseline Characteristics

Among the 136 randomized participants, 124 completed the 16-week study (65 placebo and 59 liraglutide). Complete data on the diverse measurements of gastrointestinal functions are available for 121 participants. Baseline characteristics were not significantly different between the two treatment groups (Table 13).

End-of-study weight loss >4 kg was achieved by 71% of the liraglutide group compared to 16% of placebo group.

Univariate Predictors and Factors Associated with Weight Loss in all Patients

Table 14 shows univariate predictors measured at baseline and week 5 of the study along with factors measured at week 16 of the study associated with weight loss of more >4 kg at 16 weeks in all patients. Demographic parameters such as sex, baseline BMI, baseline serum glucose, and age as well as TCF7L2 and GLPIR genotype variation were not significant predictors of weight loss >4 kg at 16 weeks.

### Baseline Predictors

As expected, liraglutide treatment alone was associated with OR=12.9 (95% CI: 5.53 to 31.11; P<0.001) of inducing weight loss of >4 kg at 16 weeks compared to placebo treatment. Total calories consumed during an ad libitum buffet meal and maximum tolerated calories consumed during nutrient drink test were both significant baseline predictors of weight loss of >4 kg at 16 weeks (Table 14). A 50-minutes of change in GET_{1/2} at baseline was a numerically but not statistically significant predictor of weight loss >4 kg; the OR was 1.71 (95% CI: 0.83 to 3.49; P=0.144). Fasting and mean postprandial PYY had no significant utility in prediction of weight loss >4 kg at 16 weeks of treatment.

### Week 5 Predictors

GET_{1/2} was a significant predictor of weight loss >4 kg at 16 weeks, with an OR=2.87 (95% CI: 1.86 to 4.43; P<0.001) for 50 minutes of change and had an area under the receiver operator characteristics curve (AUROC) of 0.77. Change in GET_{1/2} from baseline to week 5 was also a significant predictor with an OR=3.25 (95% CI: 2.01 to 5.54; P<0.001) for 50 minutes of change and with an AUROC-0.78. An absolute weight loss of 1 kg from baseline to week 5 had an OR=4.45 (95% CI: 2.55 to 7.78; P<0.001) and AUROC=0.96.

### Week 16 Associated Factors

Total calories consumed during an ad libitum buffet meal at 16 weeks were significantly associated with weight loss >4 kg at 16 weeks [OR=0.7 (95% CI: 0.58 to 0.83; P<0.001)]. Fasting and mean postprandial peptide YY at the 16-weeks nutrient drink test were not associated with weight loss of >4 kg at week 16 of the study.

Univariate Predictors and Factors Associated with Weight Loss in Liraglutide Arm

GET_{1/2} measured at baseline and week 16 were not significant predictors or associated factors with weight loss of >4 kg at 16 weeks. GET_{1/2} at week 5 had an OR=1.6 (95% CI: 0.91 to 2.83; P=0.103) for 50 minutes of change. However, total calories consumed during an ad libitum buffet meal at 16 weeks were significantly associated with weight loss >4 kg at 16 weeks in the liraglutide group, with an OR=0.68 (95% CI: 0.53 to 0.87; P=0.0019).

### Multivariable Logistic Regression Analysis

The final multivariable model using baseline variables to predict weight loss at 16 weeks of >4 kg included liraglutide treatment and total calories consumed during an ad libitum buffet meal at baseline. This model had an AUROC=0.87 (95% CI: 0.81 to 0.92).

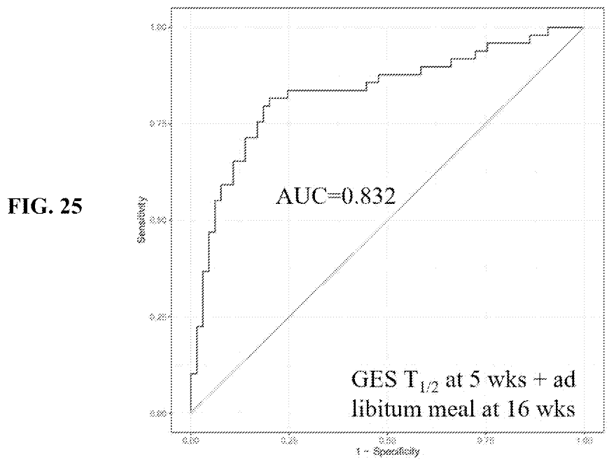

Using GET_{1/2} at 5 and 16 weeks and total kcal intake during ad libitum meal at 16 weeks in a multivariable model to identify weight loss of >4 kg at 16 weeks among all study subjects revealed two factors that were significant in the final parsimonious model: GET_{1/2} at 5 weeks (OR=2.5; 95% CI: 1.57 to 3.99) for 50 minutes of change and kcal intake during ad libitum meal at 16 weeks (OR=0.721; 95% CI: 0.602 to 0.864) for 100 kcal of change. The AUROC for this multivariable model was 0.832 (FIG. 25).

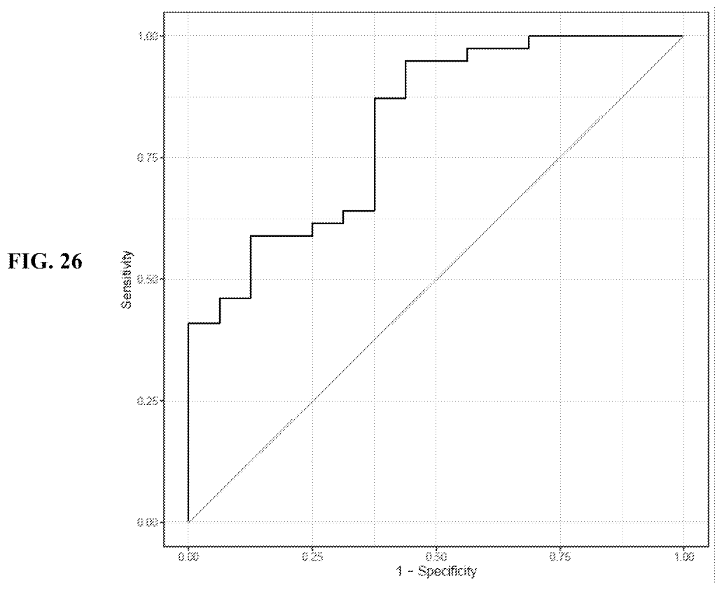

The ROC curve evaluating weight loss of >4 kg at 16 weeks, limited to the liraglutide group only using baseline GET_{1/2}, week 5 GET_{1/2}. and kcal intake during ad libitum meal at 16 weeks, showed an AUROC of 0.814 (FIG. 26). The parsimonious model identified kcal intake during ad libitum meal at 16 weeks as the only individual parameter associated with weight loss >4 kg, with an AUROC-0.757.

Average weight loss on liraglutide was 5.8 kg; >4 kgs was achieved by 71% of liraglutide arm and 16% of placebo arm. Full data on functional measurements are available for 121 participants. Three parameters were univariately associated with >4 kg weight loss; 2 factors remained significant on multivariate analysis (Table 15) leading to the final parsimonious models that identified factors associated with >4 kg weight loss for all patients in the 2 treatment arms (Table 13): GES T_{1/2} at 5 weeks (OR=2.505; 95% CI: 1.57 to 3.997) and kcal intake at ad libitum meal at 16 weeks (OR=0.721; 95% CI: 0.602 to 0.864). The area under the ROC curve (AUROC) for this model was 0.832 (FIG. 25). One variable was identified in the final model for the liraglutide group alone: ad libitum meal kcal intake at 16 weeks (OR=0.679; 95% CI: 0.532 to 0.867). The AUROC was 0.757.

### Conclusion

This analysis showed that, among patients with obesity attempting weight loss with either liraglutide or placebo treatment, a delay in gastric emptying at 5 weeks predicted weight loss >4 kg at 16 weeks, as evident on univariate analysis and in the parsimonious model. Moreover, the AUROC curve for the parsimonious model for all patients was 0.832 compared to 0.77 for GET1/2 at 5 weeks on univariate analysis. Therefore, retardation of GET1/2 at 5 weeks remains a relevant predictor of weight loss at 16 weeks without consideration of the kcal ingested at ad libitum meal at 16 weeks. In addition, energy intake by ad libitum meal at 16 weeks was associated with weight loss in all patients. This latter observation reflects an association of the effects of liraglutide on appetite and clearly does not provide prediction of weight loss response to liraglutide treatment.

When considering variables of those treated only with liraglutide, weight loss >4 kg at 16 weeks was best associated with reduction in kcal intake during ad libitum meal at 16 weeks as well as GET_{1/2} at 5 weeks (AUROC of 0.63) and weight loss >1 kg in the first 5 weeks (AUROC=0.96).

The parsimonious model using backward selection for the liraglutide group identified only one significant variable associated with weight loss of >4 kg at 16 weeks, which was the kcal intake at ad libitum meal at 16 weeks. However, the GET_{1/2} at baseline and 5 weeks marginally enhanced the prediction of weight loss. The observation of the impact of meal kcal intake in predicting weight loss confirms prior research by another group.²⁰ Thus, this study suggests that further research is necessary to characterize the associations with central mechanisms in addition to satiation mediated peripherally by satiation-associated hormones or gastric functions. Although most subjects that achieved weight loss >4 kg were in the liraglutide arm, the assessment of predictive value confined to the liraglutide group and dichotomizing continuous variables (> or <4 kg) reduced statistical power to identify predictors of weight loss in the liraglutide group alone.²⁷ Note that the odds ratio for GET1/2 at 5 weeks for the liraglutide group was 1.6 and the 95% CI: 0.91 to 2.83; P=0.103 for 50 minutes of change. Of note, demographic parameters such as sex, baseline BMI, baseline serum glucose, and age were of no predictive utility.

In an earlier study, gastric emptying was not significantly associated with weight loss in patients on liraglutide.²⁰ In fact, other studies have suggested that liraglutide was not associated with delayed gastric emptying.^{14, 28, 29} However, gastric emptying in those studies was measured using a suboptimal methodology that utilizes acetaminophen absorption, which is an indirect way of assessing liquid emptying rather than solid emptying.³⁰ One of the strengths of the methods used in this Example is the use of gastric emptying scintigraphy based on a 320 kcal egg meal, given the different rates of emptying between liquids and solids.³¹ Other strengths of this study include a much larger sample size of 121 patients analyzed compared to 61 patients²⁰, and the longer treatment span of 16 weeks compared to 6 weeks in the most recent study that tried to identify a short-term biomarker for the effectiveness of liraglutide.²⁰

This study has important clinical implications, specifically that weight loss of >1 kg at 5 weeks and gastric emptying retardation at 5 weeks can serve as useful and valid predictors of weight loss over 16 weeks and may facilitate assessment of the benefit-to-cost ratio of this relatively expensive treatment that requires daily subcutaneous injection.

In summary, gastric emptying retardation at 5 weeks predicts weight loss and decreased kcal intake measured by ad libitum meal is associated with increased odds of weight loss >4 kg in response to liraglutide treatment in obesity.

TABLE 13
Demographics and baseline measurements of gastrointestinal
functions in two treatment groups.

| | Placebo, | | Liraglutide, | |
|---|---|---|---|---|
| Data show median and IQR | n = 69 | | n = 67 | |
| Age, y | 37.2 | (29.3, 45.2) | 42 | (32, 51) |
| Sex (% female) | 85.5% | | 88.1% | |
| Race, % white | 94.2% | | 89.6% | |
| BMI, kg/m² | 35.6 | (33.1, 39.7) | 35.9 | (32.6, 40.2) |
| Baseline weight, kg | 100.0 | (92.4, 114.9) | 103.1 | (89.1, 111.9) |
| Baseline Gastric emptying T_{1/2}, min | 108.0 | (93.1, 128.6) | 117.2 | (97.5, 140.0) |
| Baseline gastric fasting volume, mL | 200.8 | (179.3, 231.2) | 200.4 | (179.3, 231.2) |
| Baseline gastric postprandial vol., mL | 587.0 | (525.4, 678.0) | 593.5 | (489.3, 648.6) |
| Baseline gastric accommodation vol, | 378.5 | (322.5, 455.9) | 377.1 | (322.6, 445.3) |
| mL | | | | |
| Baseline satiation volume to fullness | 756 | (535.5, 945.0) | 693 | (567.0, 871.0) |
| (VTF), mL | | | | |
| Baseline satiation maximum tolerated | 1244.3 | (995.4, 1493.1) | 1244.3 | (995.4, 1244.3) |
| (MTV), mL | | | | |
| Baseline VAS aggregate score | 206.0 | (151.5, 256.5) | 204.0 | (156.0, 253.0) |
| Baseline ad libitum meal total calories | 878.6 | (708.2, 1151.1) | 829.5 | (665.7, 1088.5) |

TABLE 14
Odds ratios (OR) and 95% confidence intervals from univariate analysis for factors
measured at baseline, week 5, and week 16 of the study to achieve weight loss of more than
4 kilograms at 16 weeks of the study. OR is reported for 10-and 50-minutes of change of GE
T_{1/2} and for 100 kcal of change in calorie intake at ad libitum meal while achieving weight loss
>4.0 kg at 16 weeks from univariate logistic regression analyses, based on all 121 patients
(liraglutide and placebo groups) and on 60 patients in the liraglutide arm alone.

| | 95% CI | | | |
|---|---|---|---|---|
| Odds ratio | Lower | Upper | P-value | c-statistic |

ALL PATIENTS
Baseline Variables

| Liraglutide | | 12.9 | 5.35 | 31.1 | <0.001 | 0.78 |
|---|---|---|---|---|---|---|
| rs6923761 genotype | | 1.37 | 0.66 | 2.8 | 0.3967 | 0.54 |
| rs 7903146 genotype | | 1.02 | 0.50 | 2.11 | 0.9509 | 0.50 |
| Buffet meal total calories-100 kcal | | 0.78 | 0.69 | 0.90 | <0.001 | 0.70 |
| Nutrient drink test maximum calories- | | 0.88 | 0.79 | 0.90 | 0.0251 | 0.62 |
| 100 kcal | | | | | | |
| Fasting PYY-10 pg/mL | | 1.01 | 0.92 | 1.11 | 0.83 | 0.50 |
| Mean post prandial PYY-10 pg/mL | | 1 | 0.92 | 1 | 0.9867 | 0.50 |
| Gastric emptying T_{1/2} | 10 minutes | 1.11 | 0.96 | 1.28 | 0.144 | 0.57 |
| | 50 minutes | 1.71 | 0.83 | 3.49 | | |

Week 5 variables

| | | | | | | |
|---|---|---|---|---|---|---|
| Gastric emptying T_{1/2} | 10 minutes | 1.23 | 1.13 | 1.35 | <0.001 | 0.77 |
| | 50 minutes | 2.866 | 1.856 | 4.425 | | |

Change from baseline to week 5

| Body weight-1 Kg | | 4.45 | 2.55 | 7.78 | <0.001 | 0.96 |
|---|---|---|---|---|---|---|
| Gastric emptying T_{1/2} | 10 minutes | 1.27 | 1.15 | 1.41 | <0.001 | 0.78 |
| | 50 minutes | 3.25 | 2.01 | 5.54 | | |

Week 16 variables

| Fasting PYY-10 pg/mL | | 0.99 | 0.87 | 1.13 | 0.8875 | 0.51 |
|---|---|---|---|---|---|---|
| Mean post prandial PYY-10 pg/mL | | 1.08 | 0.98 | 1.19 | 0.128 | 0.57 |
| Gastric emptying T_{1/2} | 10 minutes | 1.23 | 1.11 | 1.37 | <0.001 | 0.72 |
| | 50 minutes | 2.86 | 1.66 | 4.91 | | |

| | | | | | |
|---|---|---|---|---|---|
| Buffet meal total calories-100 kcal | 0.70 | 0.58 | 0.83 | <0.001 | 0.76 |

LIRAGLUTIDE ARM N = 60
Baseline Variable

| | | | | | | |
|---|---|---|---|---|---|---|
| Gastric emptying T_{1/2} | 10 minutes | 1.08 | 0.85 | 1.37 | 0.525 | 0.56 |
| | 50 minutes | 1.47 | 0.45 | 4.80 | | |

Week 5 variable

| | | | | | | |
|---|---|---|---|---|---|---|
| Gastric emptying T_{1/2} | 10 minutes | 1.10 | 0.98 | 1.23 | 0.1029 | 0.63 |
| | 50 minutes | 1.60 | 0.91 | 2.83 | | |

Week 16 variables

| | | | | | | |
|---|---|---|---|---|---|---|
| Gastric emptying T_{1/2} | 10 minutes | 1.09 | 0.95 | 1.26 | 0.1944 | 0.64 |
| | 50 minutes | 1.57 | 0.78 | 3.12 | | |

| | | | | | |
|---|---|---|---|---|---|
| Buffet meal total calories-100 kcal | 0.68 | 0.53 | 0.87 | 0.0019 | 0.76 |

TABLE 15
Odds ratios for 50-minute retardation of GE T_{1/2} or reduced intake by 100 kcal at ad libitum
meal while achieving weight loss >4.0 kg at 16 weeks from univariate and multivariate logistic
regression analyses, based on all 121 patients (liraglutide and placebo groups).

| | Univariate analysis | | | | | Multivariate analysis | | | | |
|---|---|---|---|---|---|---|---|---|---|---|
| | Odds | 95% CI | | P- | c- | Odds | 95% CI | | P- | c- |
| | ratio | Lower | Upper | value | statistic | ratio | Lower | Upper | value | statistic |
| Baseline GE T_{1/2} | 1.705 | 0.834 | 3.488 | 0.1438 | 0.569 | — | — | — | — | 0.832 |
| GE T_{1/2} at 5 weeks | 2.866 | 1.856 | 4.425 | <0.0001 | 0.772 | 2.505 | 1.57 | 3.997 | 0.0001 | |
| GE T_{1/2} at 16 weeks | 2.857 | 1.662 | 4.911 | 0.0001 | 0.724 | — | — | — | — | |
| Meal total kcal at | 0.695 | 0.584 | 0.828 | <0.0001 | 0.758 | 0.721 | 0.602 | 0.864 | 0.0004 | |
| 16 weeks | | | | | | | | | | |

### REFERENCES

1. Ng M, Fleming T, Robinson M, et al. Global, regional, and national prevalence of overweight and obesity in children and adults during 1980-2013: a systematic analysis for the Global Burden of Disease Study 2013. The lancet 2014; 384: 766-781.
2. Heymsfield S B and Wadden T A. Mechanisms, pathophysiology, and management of obesity. New England Journal of Medicine 2017; 376: 254-266.
3. Webb V L and Wadden T A. Intensive lifestyle intervention for obesity: principles, practices, and results. Gastroenterology 2017; 152: 1752-1764.
4. Pi-Sunyer X, Astrup A, Fujioka K, et al. A randomized, controlled trial of 3.0 mg of liraglutide in weight management. New England Journal of Medicine 2015; 373: 11-22.
5. Khera R, Murad M H, Chandar A K, et al. Association of pharmacological treatments for obesity with weight loss and adverse events: a systematic review and meta-analysis. Jama 2016; 315: 2424-2434.
6. Khera R, Pandey A, Chandar A K, et al. Effects of weight-loss medications on cardiometabolic risk profiles: a systematic review and network meta-analysis. Gastroenterology 2018; 154: 1309-1319. e1307.
7. Vosoughi K, Atieh J, Khanna L, et al. Association of glucagon-like peptide 1 analogs and agonists administered for obesity with weight loss and adverse events: a systematic review and network meta-analysis. EClinicalMedicine 2021; 42: 101213.
8. Maselli D B and Camilleri M. Effects of GLP-1 and its analogs on gastric physiology in diabetes mellitus and obesity. Adv Exp Med Biol 2020; 1307: 171-192.
9. Cifuentes L, Camilleri M and Acosta A. Gastric Sensory and Motor Functions and Energy Intake in Health and Obesity-Therapeutic Implications. Nutrients 2021; 13: 1158.
10. Umapathysivam M M, Lee M Y, Jones K L, et al. Comparative effects of prolonged and intermittent stimulation of the glucagon-like peptide 1 receptor on gastric emptying and glycemia. Diabetes 2014; 63: 785-790. 2013 Oct. 4. DOI: 10.2337/db13-0893.
11. Horowitz M, Flint A, Jones K L, et al. Effect of the once-daily human GLP-1 analogue liraglutide on appetite, energy intake, energy expenditure and gastric emptying in type 2 diabetes. Diabetes research and clinical practice 2012; 97: 258-266.
12. Farr O M, Sofopoulos M, Tsoukas M A, et al. GLP-1 receptors exist in the parietal cortex, hypothalamus and medulla of human brains and the GLP-1 analogue liraglutide alters brain activity related to highly desirable food cues in individuals with diabetes: a crossover, randomised, placebo-controlled trial. Diabetologia 2016; 59: 954-965.
13. Ten Kulve J S, Veltman D J, van Bloemendaal L, et al. Endogenous GLP1 and GLP1 analogue alter CNS responses to palatable food consumption. Journal of Endocrinology 2016; 229: 1-12.
14. Van Can J, Sloth B, Jensen C, et al. Effects of the once-daily GLP-1 analog liraglutide on gastric emptying, glycemic parameters, appetite and energy metabolism in obese, non-diabetic adults. International journal of obesity 2014; 38: 784-793.
15. Harder H, Nielsen L, Thi T D, et al. The effect of liraglutide, a long-acting glucagon-like peptide 1 derivative, on glycemic control, body composition, and 24-h energy expenditure in patients with type 2 diabetes. Diabetes care 2004; 27: 1915-1921.
16 Halawi H, Khemani D, Eckert D, et al. Effects of liraglutide on weight, satiation, and gastric functions in obesity: a randomised, placebo-controlled pilot trial. The Lancet Gastroenterology & Hepatology 2017; 2: 890-899.
17. Maselli D, Atieh J, Clark M M, et al. Effects of liraglutide on gastrointestinal functions and weight in obesity: a randomized clinical and pharmacogenomic trial. Obesity 2022, in press.
18. Delgado-Aros S, Kim D-Y, Burton D D, et al. Effect of GLP-1 on gastric volume, emptying, maximum volume ingested, and postprandial symptoms in humans. American Journal of Physiology-Gastrointestinal and Liver Physiology 2002; 282: G424-G431.
19. Flint A, Kapitza C and Zdravkovic M. The once-daily human GLP-1 analogue liraglutide impacts appetite and energy intake in patients with type 2 diabetes after short-term treatment. Diabetes, Obesity and Metabolism 2013; 15: 958-962.
20. Saxena A R, Banerjee A, Corbin K D, et al. Energy intake as a short-term biomarker for weight loss in adults with obesity receiving liraglutide: A randomized trial. Obesity science & practice 2021.
21. Camilleri M, Iturrino J, Bharucha A E, et al. Performance characteristics of scintigraphic measurement of gastric emptying of solids in healthy participants. Neurogastroenterology & Motility 2012; 24: 1076-e1562.
22. Zigmond A S and Snaith R P. The hospital anxiety and depression scale. Acta psychiatrica scandinavica 1983; 67: 361-370.
23. Bush K, Kivlahan D R, McDonell M B, et al. The AUDIT alcohol consumption questions (AUDIT-C): an effective brief screening test for problem drinking. Archives of internal medicine 1998; 158: 1789-1795.
24. Yanovski S Z, Marcus M D, Wadden T A, et al. The questionnaire on eating and weight patterns-5 (QEWP-5): An updated screening instrument for binge eating disorder. The International journal of eating disorders 2015; 48: 259.
25. Chial H, Camilleri C, Delgado-Aros S, et al. A nutrient drink test to assess maximum tolerated volume and postprandial symptoms: effects of gender, body mass index and age in health. Neurogastroenterology & Motility 2002; 14: 249-253.
26. Bouras E, Delgado-Aros S, Camilleri M, et al. SPECT imaging of the stomach: comparison with barostat, and effects of sex, age, body mass index, and fundoplication. Gut 2002; 51: 781-786.
27. Royston P, Altman D G and Sauerbrei W. Dichotomizing continuous predictors in multiple regression: a bad idea. Statistics in medicine 2006; 25: 127-141.
28. Frandsen C S, Dejgaard T F, Andersen H U, et al. Liraglutide as adjunct to insulin treatment in type 1 diabetes does not interfere with glycaemic recovery or gastric emptying rate during hypoglycaemia: A randomized, placebo-controlled, double-blind, parallel-group study. Diabetes, Obesity and Metabolism 2017; 19: 773-782.
29. Jelsing J, Vrang N, Hansen G, et al. Liraglutide: short-lived effect on gastric emptying-long lasting effects on body weight. Diabetes, Obesity and Metabolism 2012; 14: 531-538.
30. Kim D-Y, Myung S-J and Camilleri M. Novel testing of human gastric motor and sensory functions: rationale, methods, and potential applications in clinical practice. The American journal of gastroenterology 2000; 95: 3365-3373.
31. Abell T L, Camilleri M, Donohoe K, et al. Consensus recommendations for gastric emptying scintigraphy: a joint report of the American Neurogastroenterology and Motility Society and the Society of Nuclear Medicine. Journal of nuclear medicine technology 2008; 36: 44-54.

### Numbered Embodiments of the Disclosure

Other subject matter contemplated by the present disclosure is set out in the following numbered embodiments:

1. A method for treating obesity and/or one or more obesity-related co-morbidities in a mammal, the method comprising: (a) detecting the presence of a plurality of single nucleotide polymorphisms (SNPs) in a sample obtained from a mammal suffering from obesity, wherein the plurality of SNPs is selected from the group consisting of rs1664232, rs11118997, rs9342434, rs2335852, rs11020655, rs1885034, rs7277175, rs6923761, rs7903146, rs17782313, rs3813929, rs1047776 and any combination thereof; and (b) administering a GLP-1 agonist to the subject when the plurality of SNPs are detected in the sample, thereby treating the obesity and/or the one or more obesity-related co-morbidities.

2. The method of embodiment 1, wherein the plurality of SNPs comprises rs1047776, rs17782313 and rs3813929.

3. The method of embodiment 1, wherein the plurality of SNPs comprises rs11118997, rs1664232, rs6923761, rs9342434, rs2335852, rs11020655 and rs1885034.

4. The method of embodiment 1, wherein the plurality of SNPs comprises rs11118997, rs1664232, rs6923761, rs9342434, rs2335852, rs1885034, rs11020655, rs1047776, rs17782313 and rs3813929.

5. The method of embodiment 1, wherein the plurality of SNPs comprises rs1664232, rs11118997, rs9342434, rs2335852, rs11020655, rs1885034 and rs7277175.

6. The method of embodiment 1, wherein the plurality of SNPs comprises rs1664232, rs11118997, rs9342434, rs2335852, rs11020655, rs1885034, rs7277175, rs7903146 and rs6923761.

7. The method of embodiment 1, wherein the plurality of SNPs comprises rs1664232, rs11118997, rs9342434, rs2335852, rs11020655, rs1885034, rs7277175, rs7903146, rs6923761, rs1047776, rs17782313 and rs3813929.

8. The method of any one of embodiments 1-7, wherein the detecting is performed using an amplification, hybridization and/or sequencing assay.

9. The method of any one of embodiments 1-8, wherein the mammal suffering from obesity is a human.

10. The method of any one of embodiments 1-9, wherein the sample is selected from the group consisting of a blood sample, a saliva sample, a urine sample, a breath sample, and a stool sample.

11. The method of any one of embodiments 1-10, wherein the sample is a blood sample.

12. The method of any one of embodiments 1-11, wherein the GLP-1 agonist is selected from the group consisting of exenatide, liraglutide and semaglutide.

13. The method of any one of embodiments 1-12, wherein the GLP-1 agonist is liraglutide.

14. The method of any one of embodiments 1-13, further comprising assessing gastric motor function of the mammal.

15. The method of embodiment 14, wherein assessing the gastric motor function of the mammal comprises measuring the gastric emptying of the mammal.

16. The method of embodiment 15, wherein a delay in gastric emptying for the mammal as compared to gastric emptying in a control selects the mammal for treatment with the GLP-1 agonist.

17. The method of any one of the above embodiments, wherein the one or more co-morbidities are selected from the group consisting of hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis and atherosclerosis (coronary artery disease and/or cerebrovascular disease).

18. A method for assaying a sample obtained from a mammal suffering from obesity and/or one or more obesity-related co-morbidities, the method comprising detecting the presence of a plurality of single nucleotide polymorphisms (SNPs) in a sample obtained from the mammal, wherein the plurality of SNPs are selected from the group consisting of rs1664232, rs11118997, rs9342434, rs2335852, rs11020655, rs1885034, rs7277175, rs6923761, rs7903146, rs17782313, rs3813929, rs1047776 and any combination thereof.

19. The method of embodiment 18, wherein the plurality of SNPs comprises rs1047776, rs17782313 and rs3813929.

20. The method of embodiment 18, wherein the plurality of SNPs comprises rs11118997, rs1664232, rs6923761, rs9342434, rs2335852, rs11020655 and rs1885034.

21. The method of embodiment 18, wherein the plurality of SNPs comprises rs11118997, rs1664232, rs6923761, rs9342434, rs2335852, rs1885034, rs11020655, rs1047776, rs17782313 and rs3813929.

22. The method of embodiment 18, wherein the plurality of SNPs comprises rs1664232, rs11118997, rs9342434, rs2335852, rs11020655, rs1885034 and rs7277175.

23. The method of embodiment 18, wherein the plurality of SNPs comprises rs1664232, rs11118997, rs9342434, rs2335852, rs11020655, rs1885034, rs7277175, rs7903146 and rs6923761.

24. The method of embodiment 18, wherein the plurality of SNPs comprises rs1664232, rs11118997, rs9342434, rs2335852, rs11020655, rs1885034, rs7277175, rs7903146, rs6923761, rs1047776, rs17782313 and rs3813929.

25. The method of any one of embodiments 18-24, wherein the detecting is performed using an amplification, hybridization and/or sequencing assay.

26. The method of any one of embodiments 18-25, wherein the mammal suffering from obesity is a human.

27. The method of any one of embodiments 18-26, wherein the sample is selected from the group consisting of a blood sample, a saliva sample, a urine sample, a breath sample, and a stool sample.

28. The method of any one of embodiments 18-27, wherein the sample is a blood sample.

29. A system for determining an obesity phenotype of a mammal suffering from obesity, the system comprising: (a) one or more processors; (b) one or more memories operatively coupled to at least one of the one or more processors and having instructions stored thereon that, when executed by at least one of the one or more processors, cause the system to: (i) identify the presence, absence or level of a plurality of gastrointestinal (GI) peptides, a plurality of metabolites, and/or a plurality of genetic variants in a sample obtained from a mammal suffering from obesity, thereby generating an analyte signature for the sample; (ii) populate a predictive machine learning model with the analyte signature of step (i); and (iii) utilize the predictive machine learning model to predict an obesity phenotype of the mammal suffering from obesity based on the analyte signature of the sample; and (c) one or more instruments in communication with at least one of the one or more processors, wherein the instruments, upon receipt of instructions sent by the at least one of the one or more processors, perform steps (i)-(iii).

30. The system of embodiment 29, wherein the predictive machine learning model is selected from the group consisting of least absolute shrinkage and selection operator (LASSO) regression, a classification and regression tree (CART) model, and a gradient boosting machine (GBM) model.

31. The system of embodiment 29 or 30, wherein the obesity phenotype is selected from the group consisting of abnormal satiation (hungry brain), abnormal satiety (hungry gut); hedonic eating (emotional hunger) and slow metabolism (slow burn).

32. The system of any one of embodiments 29-31, wherein utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

33. The system of any one of embodiments 29-32, wherein utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

34. The system of any one of embodiments 29-33, wherein the mammal suffering from obesity is a human.

35. The system of any one of embodiments 29-34, wherein the sample is selected from the group consisting of a blood sample, a saliva sample, a urine sample, a breath sample, and a stool sample.

36. The system of any one of embodiments 29-35, wherein the sample is a blood sample.

37. The system of any one of embodiments 29-36, wherein the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide.

38. The system of any one of embodiments 29-37, wherein the plurality of metabolites is selected from the group consisting of a bile acid, a neurotransmitter, an amino compound and a fatty acid.

39. The system of any one of embodiments 29-37, wherein the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

40. The system of any one of embodiments 29-39, wherein the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLPIR, PLXNA1, EYS, PTPRN2, PANX1, FRMD6, PCNT and BBS1.

41. The system of any one of embodiments 29-39, wherein the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577.

42. The system of any one of embodiments 29-41, wherein the one or more memories operatively coupled to the at least one of the one or more processors and having instructions stored thereon that, when executed by at least one of the one or more processors, further cause the system to populate the predictive learning model with data concerning the gastric motor function, resting energy expenditure (REE), one or more measures of appetite, results on behavioral questionnaires or any combination thereof of the subject suffering from obesity.

43. The system of embodiment 42, wherein the gastric motor function is determined by measuring gastric emptying of the mammal.

44. The system of embodiment 43, wherein the gastric emptying is measured using scintigraphy.

45. The system of embodiment 42, wherein the REE of the mammal is measured by indirect calorimetry.

46. The system of embodiment 42, wherein the behavioral questionnaire is a Hospital Anxiety and Depression Scale (HADS) questionnaire.

47. The system of embodiment 42, wherein the one or more measures of appetite are selected from the group consisting of calories to fullness (CTF), maximum tolerated calories (MTC) and intake calories at an ad libitum buffet meal.

48. A method for treating obesity in a mammal, the method comprising: identifying the presence, absence or level of a plurality of GI peptides, a plurality of metabolites, and/or a plurality of genetic variants in a sample obtained from a mammal suffering from obesity, thereby generating an analyte signature for the sample; populating a predictive machine learning model with the analyte signature of step (a); utilizing the predictive machine learning model to predict an obesity phenotype of the mammal based on the analyte signature of the sample obtained from the mammal, wherein the obesity phenotype is selected from the group consisting of abnormal satiation (hungry brain), abnormal satiety (hungry gut); hedonic eating (emotional hunger) and slow metabolism (slow burn); and administering an intervention based on the obesity phenotype predicted in step (c).

49. The method of embodiment 48, wherein the predictive machine learning model is selected from the group consisting of least absolute shrinkage and selection operator (LASSO) regression, a classification and regression tree (CART) model, and a gradient boosting machine (GBM) model.

50. The method of embodiment 48 or 49, wherein utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

51. The method of any one of embodiments 48-50, wherein utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

52. The method of any one of embodiments 48-51, wherein the mammal suffering from obesity is a human.

53. The method of any one of embodiments 48-52, wherein the sample is selected from the group consisting of a blood sample, a saliva sample, a urine sample, a breath sample, and a stool sample.

54. The method of any one of embodiments 48-53, wherein the sample is a blood sample.

55. The method of any one of embodiments 48-54, wherein the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide.

56. The method of any one of embodiments 48-55, wherein the plurality of metabolites is selected from the group consisting of a bile acid, a neurotransmitter, an amino compound and a fatty acid.

57. The method of any one of embodiments 48-55, wherein the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma.-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

58. The method of any one of embodiments 48-57, wherein the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLPIR, PLXNA1, EYS, PTPRN2, PANX1, FRMD6, PCNT and BBS1.

59. The method of any one of embodiments 48-57, wherein the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577.

60. The method of any one of embodiments 48-59, further comprising populating the predictive learning model with data concerning the gastric motor function, resting energy expenditure (REE), one or more measures of appetite, results on behavioral questionnaires or any combination thereof of the subject suffering from obesity.

61. The method of embodiment 60, wherein the gastric motor function is determined by measuring gastric emptying of the mammal.

62. The method of embodiment 61, wherein the gastric emptying is measured using scintigraphy.

63. The method of embodiment 60, wherein the REE of the mammal is measured by indirect calorimetry.

64. The method of embodiment 60, wherein the behavioral questionnaire is a Hospital Anxiety and Depression Scale (HADS) questionnaire.

65. The method of embodiment 60, wherein the one or more measures of appetite are selected from the group consisting of calories to fullness (CTF), maximum tolerated calories (MTC) and intake calories at an ad libitum buffet meal.

66. The method of any one of embodiments 48-65, wherein the intervention is selected from the group consisting of a pharmacological intervention, a surgical intervention, a weight loss device, a diet intervention, a behavior intervention and a microbiome intervention.

67. The method of any one of embodiments 48-65, wherein the obesity phenotype is abnormal satiation (hungry brain) and the intervention is a pharmacological intervention, wherein the pharmacological intervention is phentermine-topiramate pharmacotherapy.

68. The method of any one of embodiments 48-65, wherein the obesity phenotype is abnormal satiety (hungry gut) and the intervention is a pharmacological intervention, wherein the pharmacological intervention is a GLP-1 agonist.

69. The method of embodiment 68, wherein the GLP-1 agonist is selected from the group consisting of exenatide, liraglutide and semaglutide.

70. The method of any one of embodiments 48-65, wherein the obesity phenotype is hedonic eating (emotional hunger) and the intervention is a pharmacological intervention, wherein the pharmacological intervention is naltrexone-bupropion pharmacotherapy.

71. The method of any one of embodiments 48-65, wherein the obesity phenotype is slow metabolism (slow burn) and the intervention is a pharmacological intervention, wherein the pharmacological intervention is phentermine pharmacotherapy.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed:

1. A method for treating obesity and/or one or more obesity-related co-morbidities in a mammal, the method comprising:
(a) detecting the presence of a plurality of single nucleotide polymorphisms (SNPs) in a sample obtained from a mammal suffering from obesity, wherein the plurality of SNPs is selected from the group consisting of rs1664232, rs11118997, rs9342434, rs2335852, rs11020655, rs1885034, rs7277175, rs6923761, rs7903146, rs17782313, rs3813929, rs1047776 and any combination thereof; and
(b) administering a GLP-1 agonist to the subject when the plurality of SNPs are detected in the sample, thereby treating the obesity and/or the one or more obesity-related co-morbidities.

2. The method of claim 1, wherein the plurality of SNPs comprises rs1047776, rs17782313 and rs3813929.

3. The method of claim 1, wherein the plurality of SNPs comprises rs11118997, rs1664232, rs6923761, rs9342434, rs2335852, rs11020655 and rs1885034.

4. The method of claim 1, wherein the plurality of SNPs comprises rs11118997, rs1664232, rs6923761, rs9342434, rs2335852, rs1885034, rs11020655, rs1047776, rs17782313 and rs3813929.

5. The method of claim 1, wherein the plurality of SNPs comprises rs1664232, rs11118997, rs9342434, rs2335852, rs11020655, rs1885034 and rs7277175.

6. The method of claim 1, wherein the plurality of SNPs comprises rs1664232, rs11118997, rs9342434, rs2335852, rs11020655, rs1885034, rs7277175, rs7903146 and rs6923761.

7. The method of claim 1, wherein the plurality of SNPs comprises rs1664232, rs11118997, rs9342434, rs2335852, rs11020655, rs1885034, rs7277175, rs7903146, rs6923761, rs1047776, rs17782313 and rs3813929.

8. The method of claim 7, wherein the detecting is performed using an amplification, hybridization and/or sequencing assay.

9. The method of claim 1, wherein the mammal suffering from obesity is a human.

10. The method of claim 1, wherein the sample is selected from the group consisting of a blood sample, a saliva sample, a urine sample, a breath sample, and a stool sample.

11. The method of claim 1, wherein the sample is a blood sample.

12. The method of claim 1, wherein the GLP-1 agonist is selected from the group consisting of exenatide, liraglutide and semaglutide.

13. The method of claim 1, wherein the GLP-1 agonist is liraglutide.

14. The method of claim 1, further comprising assessing gastric motor function of the mammal.

15. The method of claim 14, wherein assessing the gastric motor function of the mammal comprises measuring the gastric emptying of the mammal.

16. The method of claim 15, wherein a delay in gastric emptying for the mammal as compared to gastric emptying in a control selects the mammal for treatment with the GLP-1 agonist.

17. The method of claim 1, wherein the one or more co-morbidities are selected from the group consisting of hypertension, type 2 diabetes, dyslipidemia, obstructive sleep apnea, gastroesophageal reflux disease, weight baring joint arthritis, cancer, non-alcoholic fatty liver disease, nonalcoholic steatohepatitis and atherosclerosis (coronary artery disease and/or cerebrovascular disease).

18. A method for assaying a sample obtained from a mammal suffering from obesity and/or one or more obesity-related co-morbidities, the method comprising detecting the presence of a plurality of single nucleotide polymorphisms (SNPs) in a sample obtained from the mammal, wherein the plurality of SNPs are selected from the group consisting of rs1664232, rs11118997, rs9342434, rs2335852, rs11020655, rs1885034, rs7277175, rs6923761, rs7903146, rs17782313, rs3813929, rs1047776 and any combination thereof.

19. The method of claim 18, wherein the plurality of SNPs comprises rs1047776, rs17782313 and rs3813929.

20. The method of claim 18, wherein the plurality of SNPs comprises rs11118997, rs1664232, rs6923761, rs9342434, rs2335852, rs11020655 and rs1885034.

21. The method of claim 18, wherein the plurality of SNPs comprises rs11118997, rs1664232, rs6923761, rs9342434, rs2335852, rs1885034, rs11020655, rs1047776, rs17782313 and rs3813929.

22. The method of claim 18, wherein the plurality of SNPs comprises rs1664232, rs11118997, rs9342434, rs2335852, rs11020655, rs1885034 and rs7277175.

23. The method of claim 18, wherein the plurality of SNPs comprises rs1664232, rs11118997, rs9342434, rs2335852, rs11020655, rs1885034, rs7277175, rs7903146 and rs6923761.

24. The method of claim 18, wherein the plurality of SNPs comprises rs1664232, rs11118997, rs9342434, rs2335852, rs11020655, rs1885034, rs7277175, rs7903146, rs6923761, rs1047776, rs17782313 and rs3813929.

25. The method of claim 18, wherein the detecting is performed using an amplification, hybridization and/or sequencing assay.

26. The method of claim 18, wherein the mammal suffering from obesity is a human.

27. The method of claim 18, wherein the sample is selected from the group consisting of a blood sample, a saliva sample, a urine sample, a breath sample, and a stool sample.

28. The method of claim 18, wherein the sample is a blood sample.

29. A system for determining an obesity phenotype of a mammal suffering from obesity, the system comprising:
(a) one or more processors;
(b) one or more memories operatively coupled to at least one of the one or more processors and having instructions stored thereon that, when executed by at least one of the one or more processors, cause the system to
(i) identify the presence, absence or level of a plurality of gastrointestinal (GI) peptides, a plurality of metabolites, and/or a plurality of genetic variants in a sample obtained from a mammal suffering from obesity, thereby generating an analyte signature for the sample;
(ii) populate a predictive machine learning model with the analyte signature of step (i); and
(iii) utilize the predictive machine learning model to predict an obesity phenotype of the mammal suffering from obesity based on the analyte signature of the sample; and
(c) one or more instruments in communication with at least one of the one or more processors, wherein the instruments, upon receipt of instructions sent by the at least one of the one or more processors, perform steps (i)-(iii).

30. The system of claim 29, wherein the predictive machine learning model is selected from the group consisting of least absolute shrinkage and selection operator (LASSO) regression, a classification and regression tree (CART) model, and a gradient boosting machine (GBM) model.

31. The system of claim 29 or 30, wherein the obesity phenotype is selected from the group consisting of abnormal satiation (hungry brain), abnormal satiety (hungry gut); hedonic eating (emotional hunger) and slow metabolism (slow burn).

32. The system of claim 29, wherein utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

33. The system of claim 29, wherein utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

34. The system of claim 29, wherein the mammal suffering from obesity is a human.

35. The system of claim 29, wherein the sample is selected from the group consisting of a blood sample, a saliva sample, a urine sample, a breath sample, and a stool sample.

36. The system of claim 29, wherein the sample is a blood sample.

37. The system of claim 29, wherein the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide.

38. The system of claim 29, wherein the plurality of metabolites is selected from the group consisting of a bile acid, a neurotransmitter, an amino compound and a fatty acid.

39. The system of claim 29, wherein the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

40. The system of claim 29, wherein the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLP1R, PLXNA1, EYS, PTPRN2, PANX1, FRMD6, PCNT and BBS1.

41. The system of claim 29, wherein the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577.

42. The system of claim 29, wherein the one or more memories operatively coupled to the at least one of the one or more processors and having instructions stored thereon that, when executed by at least one of the one or more processors, further cause the system to populate the predictive learning model with data concerning the gastric motor function, resting energy expenditure (REE), one or more measures of appetite, results on behavioral questionnaires or any combination thereof of the subject suffering from obesity.

43. The system of claim 42, wherein the gastric motor function is determined by measuring gastric emptying of the mammal.

44. The system of claim 43, wherein the gastric emptying is measured using scintigraphy.

45. The system of claim 42, wherein the REE of the mammal is measured by indirect calorimetry.

46. The system of claim 42, wherein the behavioral questionnaire is a Hospital Anxiety and Depression Scale (HADS) questionnaire.

47. The system of claim 42, wherein the one or more measures of appetite are selected from the group consisting of calories to fullness (CTF), maximum tolerated calories (MTC) and intake calories at an ad libitum buffet meal.

48. A method for treating obesity in a mammal, the method comprising:
(a) identifying the presence, absence or level of a plurality of GI peptides, a plurality of metabolites, and/or a plurality of genetic variants in a sample obtained from a mammal suffering from obesity, thereby generating an analyte signature for the sample;
(b) populating a predictive machine learning model with the analyte signature of step (a);
(c) utilizing the predictive machine learning model to predict an obesity phenotype of the mammal based on the analyte signature of the sample obtained from the mammal, wherein the obesity phenotype is selected from the group consisting of abnormal satiation (hungry brain), abnormal satiety (hungry gut); hedonic eating (emotional hunger) and slow metabolism (slow burn); and
(d) administering an intervention based on the obesity phenotype predicted in step (c).

49. The method of claim 48, wherein the predictive machine learning model is selected from the group consisting of least absolute shrinkage and selection operator (LASSO) regression, a classification and regression tree (CART) model, and a gradient boosting machine (GBM) model.

50. The method of claim 48 or 49, wherein utilization of the predictive machine learning model predicts the obesity phenotype of the mammal suffering from obesity with an accuracy of at least 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

51. The method of claim 48, wherein utilization of the predictive machine learning model predicts the obesity phenotype of the mammal suffering from obesity with a precision of at least 65%, 66%, 67%, 68%, 69%, 70%, 71%, 72%, 73%, 74%, 75% 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

52. The method of claim 48, wherein the mammal suffering from obesity is a human.

53. The method of claim 48, wherein the sample is selected from the group consisting of a blood sample, a saliva sample, a urine sample, a breath sample, and a stool sample.

54. The method of claim 48, wherein the sample is a blood sample.

55. The method of claim 48, wherein the plurality of GI peptides is selected from the group consisting of ghrelin, peptide tyrosine tyrosine (PYY), cholecystokinin (CCK), glucagon-like peptide-1 (GLP-1), GLP-2, glucagon, oxyntomodulin, neurotensin, fibroblast growth factor (FGF), GIP, OXM, FGF19, FGF19, and pancreatic polypeptide.

56. The method of claim 48, wherein the plurality of metabolites is selected from the group consisting of a bile acid, a neurotransmitter, an amino compound and a fatty acid.

57. The method of claim 48, wherein the plurality of metabolites is selected from the group consisting of 1-methylhistine, serotonin, glutamine, gamma-amino-n-butyric-acid, isocaproic, allo-isoleucine, hydroxyproline, beta-aminoisobutyric-acid, alanine, hexanoic, tyrosine, phenylalanine .gamma-aminobutyric acid, acetic, histidine, LCA, ghrelin, ADRA2A, cholesterol, glucose, acetylcholine, propionic, CDCA, PYY, ADRA2C, insulin, adenosine, isobutyric, 1-methylhistidine, DCA, CCK, GNB3, glucagon, aspartate, butyric, 3-methylhistidine, UDCA, GLP-1, FTO, leptin, dopamine, valeric, asparagine, HDCA, GLP-2, MC4R, adiponectin, D-serine, isovaleric, phosphoethanolamine, CA, glucagon, TCF7L2, glutamate, hexanoic, arginine, GLCA, oxyntomodulin, 5-HTTLPR, glycine, octanoic, carnosine, GCDCA, neurotensin, HTR2C, myristic, taurine, GDCA, FGF, UCP2, norepinephrine, palmitic, anserine, GUDCA, GIP, UCP3, serotonin, palmitoleic, serine, GHDCA, OXM, GPBAR1, taurine, palmitelaidic, glutamine, GCA, FGF19, NR1H4, stearic, ethanolamine, TLCA, FGF21, FGFR4, oleic, glycine, TCDCA, LDL, elaidic, aspartic acid, TDCA, insulin, GLP-1, linoleic, sarcosine, TUDCA, glucagon, CCK, a-linolenic, proline, THDCA, amylin, arachidonic, alpha-aminoadipic-acid, TCA, pancreatic polypeptide, eicosapentaenoic, DHCA, neurotensin, docosahexaenoic, alpha-amino-N-butyric-acid, THCA, ornithine, GLP-1 receptor, triglycerides, cystathionine 1, GOAT, cystine, DPP4, lysine, methionine, valine, isoleucine, leucine, homocystine, tryptophan, citrulline, glutamic acid, beta-alanine, threonine, hydroxylysine 1, acetone, and acetoacetic acid. In some cases, an obesity analyte signature can include 1-methylhistine, serotonin, glutamine, gamma-amino-n-butyric-acid, isocaproic, allo-isoleucine, hydroxyproline, beta-aminoisobutyric-acid, alanine, hexanoic, tyrosine, and phenylalanine.

58. The method of claim 48, wherein the plurality of genetic variants comprises single nucleotide polymorphisms (SNPs) in one or more genes selected from the group consisting of HTR2C, POMC, NPY, AGRP, MC4R, GNB3, SERT, BDNF, PYY, GLP-1, GPBAR1, TCF7L2, ADRA2A, PCSK, TMEM18, SLC6A4, DRD2, UCP3, FTO, LEP, LEPR, UCP1, UCP2, ADRA2, KLF14, NPC1, LYPLAL1, ADRB2, ADRB3, GLP1R, PLXNA1, EYS, PTPRN2, PANX1, FRMD6, PCNT and BBS1.

59. The method of claim 48, wherein the plurality of genetic variants comprises two or more SNPs selected from the group consisting of rs1664232, rs11118997, rs9342434, rs2335852, rs11020655, rs1885034, rs7277175, rs6923761, rs7903146, rs1414334, rs4795541, rs1626521 and rs2075577.

60. The method of claim 48, further comprising populating the predictive learning model with data concerning the gastric motor function, resting energy expenditure (REE), one or more measures of appetite, results on behavioral questionnaires or any combination thereof of the subject suffering from obesity.

61. The method of claim 60, wherein the gastric motor function is determined by measuring gastric emptying of the mammal.

62. The method of claim 61, wherein the gastric emptying is measured using scintigraphy.

63. The method of claim 60, wherein the REE of the mammal is measured by indirect calorimetry.

64. The method of claim 60, wherein the behavioral questionnaire is a Hospital Anxiety and Depression Scale (HADS) questionnaire.

65. The method of claim 60, wherein the one or more measures of appetite are selected from the group consisting of calories to fullness (CTF), maximum tolerated calories (MTC) and intake calories at an ad libitum buffet meal.

66. The method of claim 48, wherein the intervention is selected from the group consisting of a pharmacological intervention, a surgical intervention, a weight loss device, a diet intervention, a behavior intervention and a microbiome intervention.

67. The method of claim 48, wherein the obesity phenotype is abnormal satiation (hungry brain), and the intervention is a pharmacological intervention, wherein the pharmacological intervention is phentermine-topiramate pharmacotherapy.

68. The method of claim 48, wherein the obesity phenotype is abnormal satiety (hungry gut), and the intervention is a pharmacological intervention, wherein the pharmacological intervention is a GLP-1 agonist.

69. The method of claim 68, wherein the GLP-1 agonist is selected from the group consisting of exenatide, liraglutide and semaglutide.

70. The method of claim 48, wherein the obesity phenotype is hedonic eating (emotional hunger), and the intervention is a pharmacological intervention, wherein the pharmacological intervention is naltrexone-bupropion pharmacotherapy.

71. The method of claim 48, wherein the obesity phenotype is slow metabolism (slow burn), and the intervention is a pharmacological intervention, wherein the pharmacological intervention is phentermine pharmacotherapy.
